(19) United States
(12) Patent Application Publication
(10) Pub. No.: US 2025/0270289 A1
(43) Pub. Date: Aug. 28, 2025

(54) SEQUENCE-DEFINED POLYMERS WITH ONE OR MORE AZIDES, METHODS OF MAKING, AND METHODS OF USE THEREOF

(71) Applicant: Yale University, New Haven, CT (US)

(72) Inventors: Farren Isaacs, Stamford, CT (US); Koen Vanderschuren, New Haven, CT (US); Pol Arranz-Gibert, New Haven, CT (US); Miriam Amiram, Beer-Sheva (IL)

(21) Appl. No.: 18/558,943

(22) PCT Filed: May 5, 2022
(86) PCT No.: PCT/US2022/027885

§ 371 Date: Nov. 3, 2023

Related U.S. Application Data
(60) Provisional application No. 63/184,646, filed on May 5, 2021.

(51) Int. Cl. C07K 14/78; C12Q 1/42

(52) U.S. Cl. CPC C07K 14/78; C12Q 1/42; C07K 2319/00; C12Y 301/03016

(57) ABSTRACT

Improvements and extensions of methods of making and using para-azido-phenylalanine (pAzF)-containing polypeptides, and compositions formed therefrom are provided. Disclosed improvements and extensions include increasing the purity of pAzF present in pAzF-containing polypeptides, extending the half-Life of pAzF-containing polypeptides, and methods of making phosphoramidate (pnY)-containing polypeptides.

### CROSS-REFERENCE TO RELATED APPLICATIONS

This application claims the benefit of and priority to U.S. Provisional Application No. 63/184,646 filed May 5, 2021, which is hereby incorporated by reference in its entirety.

### STATEMENT REGARDING FEDERALLY SPONSORED RESEARCH OR DEVELOPMENT

This invention was made with government support under GM125951, awarded by National Institutes of Health and under 1714860 awarded by National Science Foundation. The government has certain rights in the invention.

### REFERENCE TO SEQUENCE LISTING

The Sequence Listing submitted as a text file named “YU_8073_PCT_ST25.txt,” created on May 5, 2022, and having a size of 66,181 bytes is hereby incorporated by reference pursuant to 37 C.F.R § 1.52(e)(5).

### FIELD OF THE INVENTION

The field of the invention generally relates to sequence-defined polymers with one or more azides, methods of making, and methods use thereof.

### BACKGROUND OF THE INVENTION

Advances in the field of genetic code expansion have empowered the incorporation of over 150 nonstandard amino acids (nsAAs) in proteins (Dumas et al., Chemical Science 6, 50-69 (2015), Xiao and Schultz, Cold Spring Harb Perspect Biol 8 (2016)), aimed at introducing new chemistries to study biological systems, or expand protein function for biotechnological applications. The nsAA para-azido-phenylalanine (pAzF) was first introduced into proteins using an orthogonal translation system (OTS) based on the tyrosyl-tRNA synthetase and tRNA of Methanocaldococcus jannaschii (Chin et al., J Am Chem Soc, 124, 9026-7 (2002)). The biorthogonal azide group of pAzF offers a remarkable number of applications based on photoreactive crosslinking (Chin et al., J Am Chem Soc, 124, 9026-7 (2002)), site-specific functionalization of proteins through Staudinger ligation (Tsao et al., Chembiochem, 6, 2147-9 (2005), Tornoe et al., J Org Chem, 67, 3057-64 (2002)), copper(I)-catalyzed azide-alkyne cycloaddition (Rostovtsev et al., Angew Chem Int Ed Engl, 41, 2596-9 (2002), or strain-promoted click chemistry (Baskin et al., Proc Natl Acad Sci USA, 104, 16793-7 (2007)).

Applications include site-specific PEGylation of pharmaceuticals (Deiters et al., Bioorg Med Chem Lett, 14, 5743-5 (2004)), production of phosphotyrosine mimetics (Serwa et al., Angew Chem Int Ed Engl, 48, 8234-9 (2009)), or creation of artificial protein complexes (Worthy et al., Communications Chemistry, 2, 83 (2019)).

Although pAzF has broad usefulness for protein functionalization, its reduction to para-amino-phenylalanine (pAF) has been reported for proteins expressed in E. coli (Wang et al., Nat Chem 6, 393-403 (2014), Young et al., Journal of Molecular Biology 395, 361-374 (2010), Amiram et al., Nat Biotechnol, 33, 1272-1279 (2015), Tsao et al., Chembiochem, 6, 2147-9 (2005)), yeast (Chin et al., Science, 301, 964-7 (2003), Chen et al., JMol Biol, 371, 112-22 (2007)), and mammalian cells (Liu et al., Nat Methods, 4, 239-44 (2007)). Such reduction of the azide moiety results in heterogeneous protein products and reduces yield and purity of the desired functionalized protein. Reduction is also observed with other nsAAs containing azide groups, such as O-2-azidoethyl-tyrosine and para-azidomethyl-phenylalanine (Frost et al., Org Biomol Chem, 14, 5803-12 (2016), Zimmerman et al., Bioconjug Chem, 25, 351-61 (2014)). The challenges posed by azide reduction are further magnified when multiple instances of azide moieties are encoded in a single protein, leading to heterogeneous products. Thus, reduction of azide moieties limits their applications and a strategy to overcome this challenge is needed.

It is therefore an object of the invention to provide compositions and methods of making proteins with unreduced azide moieties.

It is also an object of the invention to provide compositions made therefrom.

### SUMMARY OF THE INVENTION

Improvements and extensions of methods of making and using para-azido-phenylalanine (pAzF)-containing polypeptides, and compositions formed therefrom are provided, and include increasing the purity of pAzF present in pAzF-containing polypeptides, extending the half-Life of pAzF-containing polypeptides, and methods of making phosphoramidate (pnY)-containing polypeptides.

Methods of restoring one or more reduced or degraded pAzF residues in a polypeptide in need thereof are provided. The methods typically include contacting the polypeptide with an effective amount of imidazole-1-sulfonyl azide (ISAz) to restore one or more of the reduced or degraded pAzF residues to pAzF in the polypeptide. The contacting typically occurs under aqueous conditions, and in the absence of organic solvents. In preferred embodiments, the conditions are not effective to limit or prevent the conversion of amines at the N-terminus and/or lysine residues to azides. In some embodiments, (i) the contacting occurs in pH of between about 6.0 and about 8.5 inclusive, or between about 6.5 and about 7.6 inclusive, or between about 7.0 and about 7.5 inclusive, or about 7.2, or 7.2; (ii) the ISAz is in about 2 to about 500 inclusive, or between about 20 and 250 inclusive, or about 200, or 200 equivalents per molecule; (iii) the contacting is carried out for about 1 to about 150 hours, or about 2 to about 100 hours, or about 5 to about 90 hours, or about 10 to about 72 hours, or about 42, 72, or 90 hours; (iv) or any combination thereof.

In some embodiments, the polypeptide includes between about 1 and about 500 residues inclusive, or any subrange or specific integer number there between, that are either pAzF or reduced or degraded pAzF. In some embodiments, at least 10, 15, 20, 25, 30, 35, 40, 45, 50, 55, 60, 65, 70, 75, 80, 85, 90, or 95 percent of the between 1 and 500 residues are reduced or degraded pAzF prior to the contacting. In some embodiment, at least 95, 90, 85, 80, 75, 70, 65, 60, 55, or 50 percent of the between 1 and 500 residues inclusive, or any subrange or specific integer number there between, are pAzF after the contacting. 10, 20, 30, 40, 50, 60, 70, 75, 80, 85, 90, 95, 96, 97, 98, 99 or 100 percent of reduced or degraded pAzF are restored to pAzF. The contacting can occur in a composition having a plurality of the polypeptide. In some embodiments the composition is heterogeneous mixture of different polypeptides having one or more reduced or degraded pAzF residues. In some embodiments, the reduced or degraded pAzF is p-amino-phenylalanine (pAF). In some embodiments, the reduced or degraded pAzF is a degradation product of phosphoramidate (pnY), for example a pnY de-phosphorylated following contact with a phosphatase.

The polypeptide can be or include an elastin-like polypeptide (ELP). The polypeptide can be a fusion protein. In some embodiments, the polypeptide includes the amino acid sequence of SEQ ID NOS:17 or 18.

In some embodiments, the methods further include modifying the pAzF residues to include one or more moieties conjugated thereto. Modifying can include, for example, a copper-catalyzed azide-alkyne cycloaddition (“click”) chemistry reaction, strain promoted azide-alkyne cycloaddition, or Staudinger ligation photocrosslinking. In some embodiments, the moiety is a lipid, for example a fatty acid such as palmitic acid.

Methods of determining the serum half-life of polypeptides, particularly those having one or more pAzF residues, preferably modified to include one or more lipids, are also provided and can be used in conjunction with the methods of restoring one or more reduced or degraded pAzF residues, or independent thereof. Typically, the methods include determining or providing: (i) the half-life of unbound polypeptide, (ii) the half-life of serum albumin, and (iii) the binding affinity between the protein and albumin. A mathematical model is provided and can be used for, e.g., determining half-life of, for example, a series of differentially lipidated polypeptides or predicating the desired number of lipid molecules to achieve a target half-life. In some embodiments, the polypeptide is a fusion protein including an ELP having one or more lipid-conjugated pAzF residues and a therapeutic protein. Exemplary, non-limiting therapeutic proteins include recombinant blood factor concentrates or substitutes, recombinant granulocyte colony stimulating factor, and asparaginase.

Methods of making a polypeptide having one or more phosphoramidate (pnY) residues are also provided and can be used in conjunction with the methods of restoring one or more reduced or degraded pAzF residues, or independent thereof. The methods typically include subjecting a polypeptide having one or more pAzF residues to a Staudinger-phosphite ligation reaction. Typically, the Staudinger-phosphite ligation reaction includes contacting the polypeptide with an effective amount of tris(4-(2,5,8,11,14-pentaoxahexadecan-16-yloxy)-5-methoxy-2-nitrobenzyl) phosphite (TNBP). The method also typically includes a deprotection reaction. The deprotection reaction can include exposing the polypeptide to UV light. Suitable UV light sources include, but are not limited to, lasers, LEDs, and sunlight. Preferably, the methods are carried out in an alkaline buffered aqueous solution.

pnY-containing polypeptides formed according to the disclosed methods can be used as target proteins in affinity binding assays, phosphatase assays, and other assays e.g., exploring the role of phosphorylated residues. For example, in some embodiments, the methods further include utilizing the pnY-polypeptide as the subject of a binding affinity assay with a test polypeptide (e.g., having a putative binding domain). In other embodiments, the methods can further include utilizing the pnY-polypeptide as the subject of a phosphatase assay with a putative or test phosphatase. Such methods may further include restoring one or more reduced or degraded para-azido-phenylalanine (pAzF) residues (pnY residues) by repeating the disclosed restorative methods to recover pAzF residues from degrated pnY residues.

Any of the disclosed methods can be used in conjunction with a method of making the pAzF-containing polypeptide including translation of mRNA encoding the polypeptide in a translation system having an aminoacyl tRNA synthetase (AARS) and a cognate tRNA that can be charged with pAzF by the AARS and whose anticodon can recognize a codon encoding the pAzF in the mRNA. In preferred embodiments, translation is in genomically recoded organism (GRO) E. coli cells expressing the translation system. In other embodiments, translation is in vitro, optionally in a GRO lysate. In some embodiments, the AARS is selected from SEQ ID NOS:1-15.

Polypeptides manufactured according any or all of the disclosed methods are also provided. In some embodiments, the polypeptide exhibits at least 75, 80, 85, 90, 95, 96, 97, 98, or 99 percent incorporation of pAzF (or pnY) at the desired locations. Pluralities of the polypeptides are also provided can be a homogeneous plurality of a single polypeptide of interest, or a heterogeneous mixture of 2, 3, 4, 5 or more different polypeptides of interest. In some embodiments, the plurality or pluralities of polypeptide(s) have least 75, 80, 85, 90, 95, 96, 97, 98, or 99 percent incorporation of pAzF (or pnY) at the desired locations across the entire plurality. Compositions including a plurality polypeptide(s) are also provided. In some embodiments, the composition is a cell lysate or subfraction thereof. In some embodiments, the composition is a pharmaceutical composition including a pharmaceutical acceptable carrier and is suitable for administration to a subject in need thereof. Thus, the compositions can be the translation system, isolated or purified polypeptides produced by the translation system, or any intermediate thereof.

### BRIEF DESCRIPTION OF THE DRAWINGS

FIG. 1A is a schematic of a reporter construct used is some of the disclosed experiments. ELP(10UAG) is a fusion protein between an ELP with 10 pentadecapeptide repeats with one UAG codon per repeat. The UAG is decoded by an OTS for pAzF. FIG. 1B is a bar graph showing quantification of nsAA at UAG codons. Time-zero is defined as the time when the culture was started. ELP(10TAG)-GFP expression was induced at 3 h. Bar graphs show peptide (peptides 1 and 2) quantities determined by high resolution mass spectrometry (HRMS). Red curve plots percentage of pAzF residues converted to pAF (right y-axis). n=3, error bars represent standard deviation.

FIG. 2A is a schematic illustrating pH-dependent reactivity of amines—with different pKa—with ISAz facilitates selective recovery of p-azido-phenylalanine (pAzF) from p-amino-phenylalanine (pAF). FIG. 2B is a line graph showing reaction of Boc-pAF, lysine(Boc), and Boc-lysine with ISAz (200 equiv.) at pH from 6.2 to 8.2 after 72 h. At pH 7.4, only 0.5% of Boc-pAF is found; at pH 7.6 and higher, it is not detected. n=3, error bars represent standard deviation. FIG. 2C is a line graph showing pH-dependence of ISAz reactivity with 2 equivalents. Reactions of Boc-pAF, lysine(Boc), and Boc-lysine with ISAz (2 equiv.) over a range of pH from 6.2 to 8.2. n=3, error bars represent standard deviation.

FIG. 3A is a bar graph showing ISAz reaction with peptide 4 under several conditions (2, 20 or 200 equiv. of ISAz at pH 7.2, and 200 equiv. ISAz at pH 6.5) after 42 and 90 h. FIG. 3B is MS/MS spectrum of the reaction product containing an additional azido group. n=3, error bars represent standard deviation. FIG. 3C is a line graph showing a time-course of peptide conversion from ELP(pAF) to ELP(pAzF). ISAz reaction with peptide using 200 equiv. of ISAz at pH 7.2. Conversion of pAF to pAzF was measured after 5, 24, 42 and 90 h. n=3, error bars represent standard deviation.

FIG. 4A is a schematic showing GFP with an N-terminal ELP containing a mixture of pAF and pAzF. pAF residues are specifically converted to pAzF using ISAz. FIG. 4B is bar graph showing recovery of pAzF in proteins with one, five or ten pAzF residues (95.8%, 97.7 and 99.0%, respectively). n=3, error bars represent standard deviation. FIG. 4C is a deconvoluted mass spectra for GFP(pAzF) before ISAz treatment (black line) and after (red line). The observed masses are shown for each peak, and the vertical dashed lines indicate the expected masses of 30066 and 30092 Da for GFP(pAF) and GFP(pAzF), respectively. No additional +26 Da increase from off-target reactivity was observed. FIG. 4D is an EIC chromatograms of BSA peptide KQTALVELLK (deamidated glutamine and an azido-lysine (m/z 585.3481, +2)) illustrating analysis of side-reactivity of ISAz. Sample not treated with ISAz and, samples treated at pH 7.2, 8.2 and 9.0 are shown. The inset is zoomed in on the indicated region.

FIG. 5A is a schematic showing site specific multi-site incorporation of pAzF at UAG codons in the genomically recoded organism (GRO). All 321 TAG codons in E. coli were genomically recoded to TAA. To create the GRO, Release Factor 1 (RF1) was deleted. The canonical amino acids and pAzF are shown as circles. The TAG codon is converted into a sense codon for multi-site incorporation of pAzF. FIG. 5B is a schematic of the ELP-protein with 10 pAzF residues. The sequence of a single ELP repeat is highlighted. FIG. 5C is a schematic showing functionalization of azido groups in ELPs through copper(I)-mediated click chemistry with palmitic acid alkyne. FIG. 5D is flow chart showing functionalized biopolymers can be characterized in mice to study impact on half-life. FIG. 5E is a bar graph showing yields in mg/L for ELP-GFP expression with 0, 1, 5, or 10 UAG codons. Yields were determined after cell lysis, and before purification of the ELP-GFP to minimize experimental biases resulting from purifications (n=3, errorbars:mean±s.d.).

FIG. 6A is a schematic representation of ELP-sfGFP reporter constructs with 1, 5, or 10 pAzF residues. Position of nsAAs are indicated. FIGS. 6B and 6C are bar graphs showing relative abundance of detected nsAAs at guest residues of ELP-units based on quantitative high-resolution MS after purification with or without ISAz treatment before (6B) or after (6C) click chemistry with FA. (n=3, errorbars:mean±s.d.). FIGS. 6D-6G are intact mass spectrometry of full length ELP(FA)-sfGFP after click-chemistry with (right peak) or without (left peak) ISAz treatment. FIG. 6H is a flow chart illustrating the distribution of FAs per protein is dependent on the availability of pAzF. A 4% chance that any UAG codon is decoded by tyrosine, instead of pAzF was assumed. Further, assuming a 28% reduction of pAzF to pAF. The mathematical description is discussed below. FIG. 6I are plots showing intact MS peak intensities of untreated ELP(10FA)GFP compared to the probability distribution for 0-10FA per protein based on the binomial distribution specified in 6H. FIG. 6J is a bar graph showing analysis of ELP-ion counts after ISAz and click chemistry, including ELP(Tyr) peptides. Each construct contains 10 ELP units that were quantified. At UAG codons, a pAzF residue was encoded, whereas other ELP units had tyrosine encoded at the guest residue. The ELP(Tyr) counts are inversely correlated with the number UAG codons (n=3, errorbars:mean±s.d.).

FIG. 7A is a bar graph showing half-life measurements in C57BL/6 mice for pure (ISAz treated) and impure (no ISAz) constructs, injected intravenously (n=4, errorbars:mean±s.d.). ELP(0FA)GFP after ISAz treatment was not measured. FIG. 7B is a bar graph showing correlation between half-life and KD. Horizontal, black dotted line shows half-life of MSA, dashed line shows model predictions. (n=4-8, error bars: mean±s.d., n.d.=not detected.) FIG. 7C is series of images showing distribution of Alexa-647 labeled ELP-GFP constructs in mouse organs at 3 h and 48 h. Data is representative of 3 independent measurements. FIG. 7D is a bar graph showing quantification of average FI-Red intensity for organs sets in panel C. (n=4, errorbars:mean±s.e.m.). Each organ set was tested with a one-way ANOVA for statistical significance. Significance is represented on the graph as *p<0.05 and **p<0.005 after multiple testing correction. FIG. 7E is a bar graph showing cytokine profile at 3 hr and 48 hr after injection. Endotoxin (100 μg) and PBS were used as positive and negative controls, respectively. Measurements under the lower limit of detection (20 pg/mL) or above the upper limit of detection (5,000 pg/mL) are plotted at the limit of detection. (n=3, errorbars:mean±s.d.). FIGS. 7F and 7G are time-course plots of ELP-GFP quantified from blood samples after IV injection. Dots represent individual measurements from GFP ELISA, and solid lines indicate the expected values based on average half-life. 7F and 7G panels show ELP-GFP without or with ISAz treatment, respectively (n=4). FIGS. 7H and 7I are time-course plots of ELP-GFP quantified from blood samples after SC injection. Dots represent individual measurements from GFP ELISA, and solid lines indicate the expected values based on average half-life. 7H and 7I panels show ELP-GFP without or with ISAz treatment, respectively (n=4). FIG. 7J is a plot showing comparison of half-life estimations derived from the IV injections and the logarithmic decrease of the SC injections. The dashed lines indicate the 35 hours half-life of mouse serum albumin (n=4, errorbars:mean±s.d.).

FIG. 8A shows a model for computational prediction of half-life as a function of K_{D} (materials and methods). The half-life values are based on empirical data for unbound ELP-GFP obtained in this work and on reference value from³⁰ for albumin half-life. FIGS. 8B and 8C plots comparing model predictions to measured ELP-GFP abundance over time. ELISA measurements are shown as •, model predictions are shown in solid lines. Comparisons are made for pure (ISAz treated) constructs (8B), and impure (no ISAz treatment) constructs (8C).

FIGS. 9A-9G are bar graphs showing quantification of Alexa647 signal for all organs normalized to signal intensity for brain. For each organ set, a one-way ANOVA test was calculated to identify statistically significant differences in mean signal intensity between treatment groups. Results are shown for brain (9A), lungs (9B), heart (9C), spleen (9D), liver (9E), kidney (9F), and blood (9G) (n=4, errorbars:mean±s.e.m.).

FIG. 10A is a schematic illustrating how pTyr participates in a three-part toolkit including “writing” by Tyr kinases, “reading” by SH2 domains, and “erasing” by phosphatases. FIG. 10B is a schematic illustrating how a pTyr mimic introduced by Staudinger-phosphite ligation interacts with SH2 domains and phosphatases.

FIG. 11A is a schematic showing how a reporter protein is generated by in vivo pAzF incorporation with an OTS, in vitro reaction with TNBP, and UV cleavage to produce the phosphoramidate (pnY). FIG. 11B is a schematic of a reaction to regenerate pAzF from pAF using ISAz.

FIG. 11C is a histogram showing the conversion yields in protein after each reaction. Quantitation performed by bottom-up MS. FIG. 11D is an image of Western blots showing epitope recognition by an antibody against phosphorylated Cav-1 (Y14) (IB:pCav) with either Tyr (Cav(Tyr)GFP) or pAzF (Cav(pAzF)GFP) starting proteins. FIG. 11E is a bottom-up MS analysis of (Cav(TAG)GFP) protein after expression, and consecutive reactions with ISAz, TNBP and UV light. The initial sample shows similar amounts of pAF and pAzF. After reaction with ISAz, the content of pAF is ˜5%. Overnight reaction with TNBP yields 5% of pAzF unreacted, producing the protected pnY moiety. Finally, deprotection of pnY using UV light is quantitative (no protected pnY-containing peptide was detected).

FIG. 11F is an image of a Western blot and SDS-PAGE data for sunlight-based cleavage of phosphoramidate protecting groups. 1-TAG Caveolin-sfGFP reacted with TNBP as described and exposed to sunlight for the stated times. Samples immunobloted for phospho-caveolin (IB:pCav) and total protein normalized with SDS-PAGE and CBB.

FIG. 12A is a schematic illustrating a fluorescence polarization assay scheme (SEQ ID NOS:41-44). FIG. 12B is a line graph illustrating fluorescence polarization binding data for peptides containing Tyr (YEEI), pTyr (pTyrEEI), and the pTyr mimetic (pnYEEI) to the Src SH2 domain. Error bars represent the s.d.

FIG. 13A is a scheme of the dephosphorylation of pnY by phosphatases. FIG. 13B is an image of Western blot illustrating phosphatase cleavage of phosphoramidate-containing caveolin protein using 10 units of CIP calf intestinal phosphatase (CIP) or 1 μL of protein Tyr phosphatase 1B (PTP1B) per 20 μL reaction (15 min). FIG. 13C is a bar graph showing relative amounts of pAF- and pAzF-containing tryptic peptides from GFP reporter (Cav(pAzF)GFP) construct after treating the sample with TCEP and subsequently with 200 eq. ISAz at pH 7.2 (n.d. means that pAzF-peptide is not detected). Error bars represent the s.d.

### DETAILED DESCRIPTION OF THE INVENTION

### I. Definitions

As used herein, the terms “transfer RNA” and “tRNA” refers to a set of genetically encoded RNAs that act during protein synthesis as adaptor molecules, matching individual amino acids to their corresponding codon on a messenger RNA (mRNA). tRNAs assume a secondary structure with four base paired stems known as the cloverleaf structure. The tRNA contains a stem and an anticodon. The anticodon is complementary to the codon specifying the tRNA's corresponding amino acid. The anticodon is in the loop that is opposite of the stem containing the terminal nucleotides. The 3′ end of a tRNA is aminoacylated by a tRNA synthetase so that an amino acid is attached to the 3′end of the tRNA. This amino acid is delivered to a growing polypeptide chain as the anticodon sequence of the tRNA reads a codon triplet in an mRNA.

As used herein, the term “anticodon” refers to a unit made up of typically three nucleotides that correspond to the three bases of a codon on the mRNA. Each tRNA contains a specific anticodon triplet sequence that can base-pair to one or more codons for an amino acid or “stop codon.” Known “stop codons” include, but are not limited to, the three codon bases, UAA known as ochre, UAG known as amber and UGA known as opal, which do not code for an amino acid but act as signals for the termination of protein synthesis. tRNAs do not decode stop codons naturally, but can and have been engineered to do so. Stop codons are usually recognized by enzymes (release factors) that cleave the polypeptide as opposed to encode an AA via a tRNA.

As used herein, the term “suppressor tRNA” refers to a tRNA that alters the reading of a messenger RNA (mRNA) in a given translation system. For example, a nonsense suppressor tRNA can read through a stop codon.

As used herein, the term “aminoacyl tRNA synthetase (AARS)” refers to an enzyme that catalyzes the esterification of a specific amino acid or its precursor to one of all its compatible cognate tRNAs to form an aminoacyl-tRNA. These charged aminoacyl tRNAs then participate in mRNA translation and protein synthesis. The AARS show high specificity for charging a specific tRNA with the appropriate amino acid. In general, there is at least one AARS for each of the twenty amino acids.

As used herein, the term “residue” refers to an amino acid that is incorporated into a protein. The amino acid may be a naturally occurring amino acid and, unless otherwise limited, may encompass known analogs of natural amino acids that can function in a similar manner as naturally occurring amino acids.

As used herein, the terms “polynucleotide” and “nucleic acid sequence” refers to a natural or synthetic molecule including two or more nucleotides linked by a phosphate group at the 3′ position of one nucleotide to the 5′ end of another nucleotide. The polynucleotide is not limited by length, and the polynucleotide can include deoxyribonucleic acid (DNA) or ribonucleic acid (RNA).

As used herein, the terms “transformation” and “transfection” refer to the introduction of a polynucleotide, e.g., an expression vector, into a recipient cell or introduction of a polynucleotide to the chromosomal DNA of the cell.

As used herein, the term “transgenic organism” refers to any organism, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. Suitable transgenic organisms include, but are not limited to, bacteria, cyanobacteria, fungi, plants and animals. The nucleic acids described herein can be introduced into the host by methods known in the art, for example infection, transfection, transformation or transconjugation.

As used herein, the term “eukaryote” or “eukaryotic” refers to organisms or cells or tissues derived from these organisms belonging to the phylogenetic domain Eukarya such as animals (e.g., mammals, insects, reptiles, and birds), ciliates, plants (e.g., monocots, dicots, and algae), fungi, yeasts, flagellates, microsporidia, and protists.

As used herein, the term “prokaryote” or “prokaryotic” refers to organisms including, but not limited to, organisms of the Eubacteria phylogenetic domain, such as Escherichia coli, Thermus thermophilus, and Bacillus stearothermophilus, or organisms of the Archaea phylogenetic domain such as, Methanocaldococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, and Aeuropyrum pernix.

As used herein, the term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids are at least 60% free, preferably 75% free, and most preferably 90% free from other associated components.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “translation system” refers to the components that facilitate incorporation of an amino acid into a growing polypeptide chain (protein). Key components of a translation system generally include at least AARS and tRNA, and may also include amino acids, ribosomes, AARS, EF-Tu, and mRNA.

As used herein, the term “orthogonal translation system (OTS)” refers to at least an AARS and paired tRNA that are both heterologous to a host or translational system in which they can participate in translation of an mRNA including at least one codon that can hybridize to the anticodon of the tRNA.

As used herein, the terms “recoded organism” and “genomically recoded organism (GRO)” in the context of codons refer to an organism in which the genetic code of the organism has been altered such that a codon has been eliminated from the genetic code by reassignment to a synonymous or nonsynonymous codon.

As used herein, the term “polyspecific” refers to an AARS that can accept and incorporate two or more different non-standard amino acids.

As used herein, the terms “protein,” “polypeptide,” and “peptide” refers to a natural or synthetic molecule comprising two or more amino acids linked by the carboxyl group of one amino acid to the alpha amino group of another. The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus.

As used herein, “standard amino acid” and “canonical amino acid” refer to the twenty amino acids that are encoded directly by the codons of the universal genetic code denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “non-standard amino acid (nsAA)” refers to any and all amino acids that are not a standard amino acid. nsAA can be created by enzymes through posttranslational modifications; or those that are not found in nature and are entirely synthetic (e.g., synthetic amino acids (sAA)). In both classes, the nsAAs can be made synthetically. WO 2015/120287 provides a non-exhaustive list of exemplary non-standard and synthetic amino acids that are known in the art (see, e.g., Table 11 of WO 2015/120287).

As used herein, “genetically modified organism (GMO)” refers to any organism whose genetic material has been modified (e.g., altered, supplemented, etc.) using genetic engineering techniques. The modification can be extrachromasomal (e.g., an episome, plasmid, etc.), by insertion or modification of the organism's genome, or a combination thereof.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product, for example a functional RNA that does not encode a protein or polypeptide (e.g., miRNA, tRNA, etc.). The term gene as used herein with reference to genomic DNA includes intervening, non-coding regions as well as regulatory regions and can include 5′ and 3′untranslated ends. The term gene as used herein with reference to recombinant expression constructs may, but need not, include intervening, non-coding regions, regulatory regions, and/or 5′ and 3′untranslated ends. Thus, with respect to a recombinant expression constructs, a gene may be only an open reading frame (ORF).

As used herein, the term “construct” refers to a recombinant genetic molecule having one or more isolated polynucleotide sequences. Genetic constructs used for transgene expression in a host organism, also referred to “expression constructs”, include in the 5′-3′ direction, a promoter sequence; a sequence encoding a gene of interest; and a termination sequence. The construct may also include selectable marker gene(s) and other regulatory elements for expression.

As used herein, the term “vector” refers to a polynucleotide capable of transporting into a cell another polynucleotide to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector.

As used herein, the term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of gene to a transcriptional control element refers to the physical and functional relationship between the gene and promoter such that the transcription of the gene is initiated from the promoter by an RNA polymerase that specifically recognizes, binds to and transcribes the DNA.

As used herein, term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, the term “promoter” refers to a regulatory nucleic acid sequence, typically located upstream (5′) of a gene or protein coding sequence that, in conjunction with various elements, is responsible for regulating the expression of the gene or protein coding sequence. These include constitutive promoters, inducible promoters, tissue- and cell-specific promoters and developmentally-regulated promoters.

As used herein, the terms “transformed,” “transgenic,” “transfected” and “recombinant” refer to a host organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

As used herein, the term “endogenous” with regard to a nucleic acid refers to nucleic acids normally present in the host.

As used here, the term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### II. Polypeptides With One or More Azides and Uses Thereof

Improved and extended pAzF-containing polypeptide compositions, and methods of making, and use thereof are provided. Improvements and extensions include increasing the purity of pAzF present in pAzF-containing polypeptides, extending the half-Life of pAzF-containing polypeptides, and methods of making pnY-containing polypeptides, each of which are discussed in more detail in the sections that follow. Any of disclosed improvements and extensions can include a method of making pAzF-containing polypeptide composition. For example, in some embodiments, pAzF-containing polypeptides are initially formed by recombinant polypeptide expression in GRO can be transformed or genetically engineered to express the orthogonal AARS-tRNA pair and an mRNA encoding the polypeptide of interest, such that pAzF precisely and programmable added at the desired location(s) during translation. Such systems and methods are also discussed in more detail below.

The disclosed improvements and extensions are modular, and thus all of the disclosed methods can be utilized alone or in any suitable combination.

In some embodiments, the methods lead to higher yields, higher purity, or a combination thereof, of the pAzF-containing polypeptide of interest.

Higher purity of the desired polypeptide can be measured as an increase in the presence of pAzF at the desired location relative to another amino acid at the desired location. The methods are able to produce high yields of biopolymers with multiple pAzF, and still maintain purity. In some embodiments, purity is between e.g., 30% and 100% inclusive, or any range of integer values, or specific integer there between. For example, in some embodiments, the purity is at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more. Purity can be determined using routine methods such as mass spectroscopy.

Higher yield of the desired polypeptide can be measured as an increase in the amount of desired protein per total protein by weight or mass, or the amount of desired protein per culture volume, relative to the same polypeptide made using conventional methods and reagents. For example, in some embodiments, the yield is increased by at least 5, 10, 15, 20, 25, 50, 75, 100, 125, 150, 175, 200, 300, 400, or 500 percent. In some embodiments, the yield is at least 5, 10, 15, 20, 25, 50, 75, or 100 mg/L.

All compositions and intermediates formed during, and as a result of, the disclosed methods are expressly provided. Thus, in some embodiments, the composition is a plurality of cells translating pAzF-containing polypeptides, a cell lysate thereof, a plurality isolated pAzF-containing polypeptides therefrom or an individual polypeptide thereof, or another composition (e.g., pharmaceutical composition) containing pAzF-containing polypeptides, optionally in an effective amount.

A. Methods of Increasing Purity of pAzF Present in Recombinant Proteins

In Examples 1-3 below, the origins of pAzF reduction were investigated and its impact on protein purity evaluated in a genomically recoded strain of E. coli (GRO), which lacks the UAG codon and release factor 1 (Isaacs et al., Science 333, 348 (2011), Lajoie et al., Science 342, 357-360 (2013)). In this system, the UAG codon was repurposed as a dedicated coding channel for pAzF. The reduction of pAzF was analyzed throughout the processes of translation and protein purification, and post-translational reduction in the cytosol was identified as the primary source of pAzF reduction.

Results show that reduction of pAzF to pAF occurs post-translationally in the cell. The cytoplasm of E. coli is known to be a reducing environment (Prinz et al., J Biol Chem, 272, 15661-7 (1997)), which impacts the redox state of certain protein residues (such as cysteines that form disulfide bonds or pAzF). Given the central importance of the cell's redox state, it would be challenging to fully prevent pAzF reduction through metabolic engineering (Lobstein et al., Microb Cell Fact, 11, 56 (2012)). Thus, to for example, maximize protein production and avoid potential growth impairment with mutagenized strains, a method of reverting the reduction of azides in vitro after protein purification is provided.

The method is a post-purification diazotransfer reaction. Although diazotransfer reactions have been described to convert amines to azides (Katritzky et al., J Org Chem, 75, 6532-9 (2010), Nyffeler et al., J Am Chem Soc, 124, 10773-8 (2002)), they typically require harsh organic solvents that are incompatible with proteins. The hydrochloric salt of imidazole-1-sulfonyl azide (ISAz) was used by Van Hest and co-workers to introduce azides onto proteins via aqueous diazotransfer, thereby converting F-amines in lysine side-chains and the α-amine at N-terminus of proteins into azides (Van et al., Bioconjug Chem, 20, 20-3 (2009)). Certain conditions allowed the introduction of azides onto proteins while retaining protein activity (Schoffelen et al., Chemical Science 2, 701-705 (2011), van Dongen et al., Bioconjugate Chemistry 20, 20-23 (2009)). The disclosed methodology is extended and improved to recover pAzF from pAF residues, while preserving the amine groups at the N-terminus and lysines. By restoring pAzF with high efficiency and accuracy, key challenges can be overcome in expressing proteins with azides for homogeneous and predictable functionalization of polypeptides with one or more pAzF residues.

The disclosed method typically includes treatment of expressed, optionally purified or otherwise isolated recombinant protein, with a diazotransfer reaction using an effective amount of imidazole-1-sulfonyl azide (ISAz) in aqueous conditions in a pH and for a length of time effective to restore pAF residues to pAzF residues in the polypeptide of interest, while also limiting or preventing conversion of other primary amines in the polypeptide of interest (e.g., N-terminus and lysines) to azides. The reaction can be carried out free from organic solvents.

In some embodiments, the pH is in the range of 6.0-8.5 inclusive, or any subrange thereof, or specific value there between. The Examples below show that the diazotransfer reaction may be less effective when the pH is lower than 7.0. The amino group in pAF reacts first, followed by the N-terminus, and then lysine side-chains at increasing pH. Thus, preferably the pH is 6.5 or higher, and a preferred range is 6.8-7.4. A particularly preferred specific value is 7.2.

The Examples below show that the desired result may be reduced when the ISAz equivalents (defined as number of ISAz molecules per pAzF/pAF residue in the reaction) are reduced to 2 (and to a lesser extent when using 20 equiv.). Thus, the method typically includes use of about 2 to about 200 equivalents of ISAz. Preferably, the diazotranfer reaction typically includes at least 2, preferably at least 20, most preferably about 200 or more equivalents of ISAz.

The reaction can be carried out for minutes, hours, or days. For example, preferably the reaction is carried out for about 1 to about 150 hours inclusive, or any subrange thereof, or specific value there between. The Examples below show that high conversions were observed at 42 h and 90 h. Thus, preferably, the diazotranfer reaction is carried out for at least 72 hours, and a preferred range is 24-96 hours, with exemplary specific times being 42, 72, and 90 hours. The diazotransfer reaction can be carried out at 10, 20, 30 and 37° C., preferably at 20° C.

In an exemplary embodiment, the method utilizes about 200 equivalents of ISAz per molecule, pH 7.2, 20° C., 72 h. In the Examples below, these conditions achieved >95% conversion of pAF to pAzF in the proteins encoded with 1, 5, or 10 nsAAs.

The aqueous solution can be any on suitable for the desired diazotransfer reaction. In the Examples below, reactions were carried out in 10×PBS (1.4 M NaCl, 0.1 M phosphate, 0.03 M KCl) or 100 mM NaPi, but these are just two non-limiting examples.

Diazotransfer reactions can be stopped by, for example, lowering the pH below the effective reaction pH or by buffer exchange. In the Examples below, formic acid was used to lower the pH and stop diazotransfer reactions. Alternatively, the reaction may be stopped by removal of ISAz via buffer exchange.

Diazotransfer reactions are ideally performed in the dark to prevent photoactivation (and reactivity) caused by light.

B. Extending the Half-Life of pAzF-containing Polypeptides

Protein and peptide therapeutics represent a versatile and fast-growing class of biological therapeutics (Fosgerau and Hoffmann, Drug Discov Today 20, 122-128 (2015), Lee et al., Int J Mol Sci 20 (2019)). These biologics are particularly attractive as potential pharmaceuticals due to their high specificity, high activity, and, in the case of peptides, have rapid tissue penetration (Fosgerau and Hoffmann, Drug Discov Today 20, 122-128 (2015)). However, barriers prevent the widespread clinical use of peptide or protein-based therapeutics (Fosgerau and Hoffmann, Drug Discov Today 20, 122-128 (2015)): (i) the need to administer them by injection, (ii) their rapid clearance by the kidneys, and (iii) their rapid proteolytic degradation. As a result, these pharmaceuticals must be frequently administered at high doses, leading to a “peak-and-valley” pharmacokinetic profile. These characteristics can negatively affect therapeutic efficacy, can cause undesirable side-effects with reduced patient compliance (Gilroy et al., J Control Release 240, 151-164 (2016), Banga, Therapeutic Peptides and Proteins, 177-227 (CRC Press, 2005), Fu and Radican, Curr Med Res Opin 25, 1413-1420 (2009), Henninot et al., J Med Chem 61, 1382-1414 (2018)) and can trigger an immune response, including the induction of a neutralizing antibody response (Baker et al., Self Nonself 1, 314-322 (2010), Descotes and Gouraud, Expert Opin Drug Metab Toxicol 4, 1537-1549 (2008)).

To address these challenges, proteins and peptides are frequently functionalized to extend their half-life and improve immunotolerance. A widely adopted strategy is the conjugation of poly(ethylene glycol) (PEG), which increases the radius of the protein and reduces proteolytic cleavage, and consequently reduces clearance (Harris and Chess, Nat Rev Drug Discov 2, 214-221 (2003)). However, the development of alternatives to PEGylation remains important, as PEGylation does not always offer the desired effect on pharmacokinetics, and in certain cases safety concerns about its immunogenicity and accumulation in tissues have been raised (Yang and Lai, Wiley Interdiscip Rev Nanomed Nanobiotechnol 7, 655-677 (2015), Kontermann, Expert Opin Biol Ther 16, 903-915 (2016), van Witteloostuijn, ChemMedChem 11, 2474-2495 (2016)). An alternative strategy is to conjugate or fuse the therapeutic protein or peptide to serum proteins with long half-lives, such as serum albumin, antibodies (e.g., full-length or fragments of IgG), or blood components such as red blood cells (Kontermann, Expert Opin Biol Ther 16, 903-915 (2016), van Witteloostuijn, ChemMedChem 11, 2474-2495 (2016)). Similarly, many approaches use chemical moieties or peptides to promote non-covalent binding interactions to the same serum proteins and complexes in order to extend half-life ((Kontermann, Expert Opin Biol Ther 16, 903-915 (2016), van Witteloostuijn, ChemMedChem 11, 2474-2495 (2016), Kontos and Hubbell, Mol Pharm 7, 2141-2147 (2010)). One effective and safe option is the use of fatty acids to promote binding to serum albumin. For example, insulin and GLP-1 conjugated with a single fatty acid are clinically used to treat diabetic patients (Knudsen et al., J Med Chem 43, 1664-1669 (2000), Agerso et al., Diabetologia 45, 195-202 (2002), Kurtzhals et al., Biochem J 312 (Pt 3), 725-731 (1995)).

A major hurdle to the development of functionalized therapeutics is to selectively and predictably modify the protein while maintaining bioactivity. Conventional strategies for PEGylation and functionalization with chemical moieties utilize chemistries that modify the target protein at their termini, or at residues with reactive side-chains (Boutureira and Bernardes, Chem Rev 115, 2174-2195 (2015)). The functionalization at C- or N-termini can be highly selective and predictable, but it can reduce bioactivity and is thus incompatible with many proteins. In contrast, modifications at reactive side-chains (e.g., cysteine or lysine) is less restrictive, but it can be difficult (or practically impossible) to identify unique reactive sites in the peptide sequence for site-specific conjugation.

To address this problem, amino acids in the protein are typically mutagenized, which can result in reduced bioactivity. Recently, nonstandard amino acids (nsAAs) have been successfully employed for modification of proteins and peptides, offering bioorthogonal chemistries for functionalization at predetermined positions within the protein (Boutureira and Bernardes, Chem Rev 115, 2174-2195 (2015), Deiters et al., Bioorg Med Chem Lett 14, 5743-5745 (2004), Axup et al., Proc Natl Acad Sci USA 109, 16101-16106 (2012)). For example, hGH and fibroblast growth factor 21 were site-specifically PEGylated, prolonging their function through extended serum half-life in clinical trials (Cho et al., Proc Natl Acad Sci USA 108, 9060-9065 (2011), Mu et al., Diabetes 61, 505-512 (2012)). In other cases, site-specific lipidation at nsAA was shown to extend half-life in mouse models (Zorzi et al., Nat Commun 8, 16092 (2017), Lim et al., J Control Release 170, 219-225 (2013)). Lipidation is an appealing alternative to PEG, which has come under scrutiny due to concerns about immunogenicity of PEG (Ganson et al., Arthritis Res Ther 8, R12 (2006), Armstrong et al., Cancer 110, 103-111 (2007)), and uncertainty about its degradation and clearance from the body (Baumann et al., Drug Discov Today 19, 1623-1631 (2014)). The use of fatty acids has clinical precedence, offers greater tunability than direct fusion to albumin, and has a well-established safety profile (Menacho-Melgar et al., J Control Release 295, 1-12 (2019)).

However, the usefulness of current lipidation strategies is constrained by two factors. First, typically only moderate half-life extensions are achieved due to weak binding of pharmaceuticals with single fatty acids to albumin. Second, the ability to identify uniquely reactive residues without impacting bioactivity remains challenging with conventional labeling strategies. Thus, these approaches have limited the versatility and tunability (e.g., customized range of half-lives) of these functionalized peptides and proteins.

A synthetic biological platform is provided that overcomes both of these limitations with a general methodology that supports tuning the half-life extension by titrating the number of fatty acids per protein, and the ability to design conjugation sites at monomeric precision permits facile screening of permissive residues to maintain bioactivity. Exemplified in Example 4-8 below, this platform can be used to biosynthesize protein-polymer fusions with sequence-defined conjugation sites for multi-site lipidation in order to extend and tailor the half-life of proteins in vivo. In the non-limiting proof-of-principle experiments below, up to 10 instances of the nsAA para-azidophenylalanine (pAzF) were encoded in elastin-like polypeptide (ELP) fusion proteins at high yields in an engineered bacterium with a recoded genome, illustrating the ability to precisely control the position and number of fatty acids per biopolymer. No elevation of pro-inflammatory cytokine levels after injection of ELP-GFP constructs was detected compared to PBS injection, whereas injection with LPS as positive control gave a clear immune response at both 3 and 48 hours. Together, these results show that fatty acid-conjugation supports half-life extension without long-term accumulation in organs, or eliciting an inflammatory response after intravenous injection.

First, a recombinant polypeptide of interest, for example an ELP, including one or more pAzF is prepared as discussed herein, optionally including a diazotransfer reaction.

The resulting polypeptide can be reacted with a fatty acid moiety such as palmitic acid alkyne using copper(I)-catalyzed azide-alkyne Huisgen cycloaddition (click-chemistry). Exemplary ELPs and fatty acids are discussed in more detail below.

It was also discovered that the number of fatty acids per protein is strongly correlated with the binding affinity to serum albumin, allowing practitioners to tune the in vivo serum half-life of proteins without it accumulating in organs or eliciting an immune response in mouse. Data also indicates that the affinity for MSA is strongly enhanced by conjugation of multiple fatty acids per protein, and confirms that the binding affinity is correlated with the number of fatty acids. These data show that the half-life of tight binding ELP-GFP constructs with multiple fatty acid conjugates approaches the half-life of MSA in mice (35 hours), and higher than the half-life of 28 hours reported for protein-MSA fusion proteins (Yang et al., Biomater Sci 6, 2092-2100 (2018)).

A model by which half-life of the protein can be determined by three parameters: (i) the half-life of unbound recombinant protein of interest (RPI), (ii) the half-life of serum albumin, and (iii) the binding affinity between the RPI and albumin is also provided.

In this model, the predicted half-life is determined by the composite clearance of the RPI, typically fused to an ELP including a lipid conjugated at one or more pAzF residues therein, and RPI bound to albumin. First, it is supposed that free and bound RPI have differential clearance rates, where the half-life of free RPI is experimentally determined from RPI with zero fatty acids (RPI(0FA)), and the half-life of bound RPI follows that of albumin. Second, the ratio of free and bound RPI is determined by the binding affinity between RPI and albumin. Collectively, these factors can be described by four ordinary differential equations:

$${r}_{1} = [ RPI ] \times [ Albumin ] \times {k}_{a}$$

$${r}_{2} = [ Complex ] \times {k}_{d}$$

$${r}_{3} = [ RPI ] \times \frac{ln ( 2 )}{{τ}_{RPI}}$$

$${r}_{4} = [ Complex ] \times \frac{ln ( 2 )}{{τ}_{Albumin}}$$

where kₘ is the association constant for binding between albumin and RPI, and k_{d} is the dissociation constant. The reaction r₁ represent the binding of RPI to albumin and is dependent on the concentrations of both. The binding is reversible, and r₂ expresses the dissociation of the complex. Furthermore, τ_{RPI} and τ_{Albumin} are the half-lives of unbound RPI(0FA) and albumin, respectively. The reactions r₃ and r₄ describe the exponential decay of unbound RPI(0FA) and albumin, where τ_{RPI} and τ_{Albumin} are their respective half-lives.

The equations in this dynamical system allow calculation of the change of the three variables [RPI], [Albumin] and [Complex]:

$$\frac{d [ RPI ]}{dt} = - {r}_{1} + {r}_{2} - {r}_{3}$$

$$\frac{d [ Albumin ]}{dt} = - {r}_{1} + {r}_{2} + {r}_{4}$$

$$\frac{d [ Complex ]}{dt} = + {r}_{1} - {r}_{2} - {r}_{4}$$

In this system, the concentration of total albumin is kept constant by reintroducing unbound albumin equal to the amount of bound albumin that was degraded.

Starting concentrations for [Albumin], [RPI] and [Complex] can be set to e.g., 250 uM, 10 uM and 0 uM, respectively. The system of differential equations can be solved in python, using scipy.integrate.odeint, at e.g., is intervals for 144 h. The half-life can be calculated using linear regression on the log transformed [RPI]ₜₒₜₐₗ (i.e., bound and unbound) after complex formation has reached equilibrium.

In the Examples below, this model was utilized to determine ELP (as the RPI) binding to albumin. By simulating the kinetics over time, the overall ELP clearance rates were calculated, and predictions made by the model were in good agreement with the empirical measurements for K_{D} and half-life. This indicates predictive capability for the half-life based on empirically determined K_{D} values, or the model can provide a target K_{D} based on the desired half-life. Together, these results also confirm that titrating the number of fatty acids allows predictable tuning of the protein half-life by modifying the binding affinity to albumin.

Thus, a method of determining the half-life of a recombinant protein of interest can include applying empirically determined K_{D} values to the above model. Alternative a method of determining a target K_{D} value can include applying a desired half-life in the above model.

### C. pnY Compositions, and Methods of Making and Use Thereof

Many organisms expand the chemical diversity available in protein synthesis using an array of post-translational modifications (PTMs), which include glycosylation, acetylation, methylation, and phosphorylation (Hornbeck et al., Nucleic acids research 40, D261-270 (2012)). Phosphorylation of serine, threonine, and tyrosine residues regulates cell-to-cell communication pathways, proliferation, differentiation, adhesion, and metabolic homeostasis (Hunter, Curr Opin Cell Biol 21, 140-146 (2009)). Tyrosine (Tyr) phosphorylation is differentially modulated across species and cell types and occurs transiently and in low abundance (Bian et al., Nat Chem Biol (2016)), necessitating the use of varied in vitro and in vivo approaches to study the mechanistic impact of individual events. In cases where the upstream regulators are known, Tyr phosphorylation can be added post-translationally through the use of in vitro kinase reactions, but these protein preparations often suffer from off-target phosphorylation and incomplete stoichiometries (Weir et al., FEBS Lett 590, 1042-1052 (2016)).

Furthermore, in vitro phosphorylation requires knowledge of a suitable upstream kinase (Bordoli et al., Cell 158, 1033-1044 (2014)). With over 16,000 phosphotyrosine (pTyr) events currently catalogued (Bian et al., Nat Chem Biol (2016), Hornbeck et al., Nucleic acids research 40, D261-270 (2012)) new tools are required for the systematic and comprehensive study of their function and tripartite regulation involving “writing” by Tyr kinases, “reading” by Src Homology 2 (SH2) domains, and “erasing” by protein Tyr phosphatases (FIG. 10A).

Phosphorylation of tyrosine cannot be effectively recapitulated with the twenty proteinogenic amino acids, however, the field of genetic code expansion facilitates access to chemical diversity by introducing nonstandard amino acids (nsAAs) into proteins (Dumas et al., Chem Sci, 6, 50-69 (2015), Haimovich et al., Nat Rev Genet 16, 501-516 (2015), Xiao and Schultz, Cold Spring Harb Perspect Biol 8 (2016)). For example, site-specific mimicry via glutamate mutations poorly mimics the charge, pKa, and geometry of pTyr (Chooi et al., J Am Chem Soc 136, 1698-1701 (2014)) and requires empirical testing for each application. In contrast, recombinant protein expression with nsAAs has permitted precise control over PTMs and offers a targeted approach for investigating the functional impact of phosphorylation residue and its higher order combinations (Dumas et al., Chem Sci, 6, 50-69 (2015), Park et al., Science 333, 1151-1154 (2011), Subramanyam et al., Proc Natl Acad Sci USA (2016)). Extensive efforts have yielded proof-of-concept studies for the incorporation of phosphoserine, phosphothreonine and pTyr (Fan et al., FEBS Letters 590, 3040-3047 (2016), Luo et al., Nat Chem Biol 13, 845-849 (2017), Park et al., Science 333, 1151-1154 (2011), Zhang et al., Nature Methods 14, 729 (2017)). Direct incorporation of pTyr has been demonstrated with two orthogonal translation systems (OTSs) derived from Methanococcus jannaschii tyrosyl-tRNA synthetase (RS), but the methods suffer from low yields and purity (Fan et al., FEBS Letters 590, 3040-3047 (2016), Luo et al., Nat Chem Biol 13, 845-849 (2017)). A complementary strategy used an evolved Methanosarcina mazei pyrrolysyl (Pyl) RS to facilitate the incorporation of a pTyr analog containing a phosphoramidate group that can be hydrolyzed and converted to pTyr. The cleavage of the phosphoramidate group requires harsh chemical treatment (0.4 M HCl, pH-1, for 48 h), which may impair the quality of the protein under study (Hoppmann et al., Nat Chem Biol 13, 842-844 (2017)).

As an alternative, several pTyr analogs have been introduced through genetic code expansion to study pTyr (Liu and Schultz, Nat Biotechnol 24, 1436-1440 (2006), Xie and Schultz, ACS Chem Biol 2, 474-478 (2007)). Examples include direct incorporation of O-sulfotyrosine (sY), p-carboxymethyl-L-phenylalanine (pCMF), and 4-phosphonomethyl-L-phenylalanine (Pmp). Another strategy involves the conversion of p-azido-L-phenylalanine (pAzF) to a phosphoramidate (pnY), which differs from pTyr only on the atom connecting the phosphorous and the aromatic ring (Bertran-Vicente et al., Journal of the American Chemical Society 136, 13622-13628 (2014a), Serwa et al., Angew Chem Int Ed Engl 48, 8234-8239 (2009)) (Table 1).

TABLE 1
Structures and properties of phosphotyrosine mimetics: O-
sulfotyrosine, p-carboxymethyl-L-phenylalanine, 4-phosphonomethyl-L-
phenylalanine and the phosphoramidate mimetic used in this work (pnY)

| | | Decrease | | | |
|---|---|---|---|---|---|
| | | in | | | |
| | | binding | | Can be | |
| | | affinity | In vivo | dephos- | |
| Compound | Compound name | with SH2 | expression | phorylated | Ref. |
| 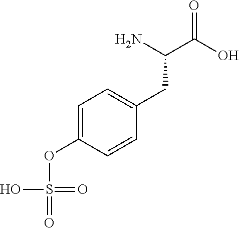 [SMILES: N[C@@H](Cc1ccc(OS(=O)(=O)O)cc1)C(=O)O] | O-sulfotyrosine (sY) | 160-fold | Y | N | Ju, et al., Mol Biosyst 9, 1829-1832 (2013) |
| 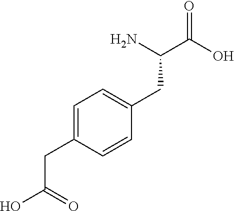 [SMILES: N[C@@H](Cc1ccc(CC(=O)O)cc1)C(=O)O] | p-carboxymethyl-L- phenylalanine (pCMF) | 450-fold | Y | N | Tong, et al., J Biol Chem 273, 20238- 20242 (1998) |
| 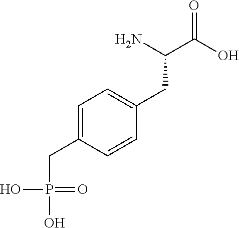 [SMILES: N[C@@H](Cc1ccc(CP(=O)(O)O)cc1)C(=O)O] | 4- phosphonomethyl- L-phenylalanine (Pmp) | 6-fold | N | N | Burke, et al., Biochemistry 33, 6490-6494 (1994) |
| 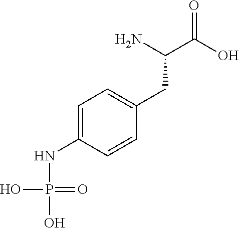 [SMILES: N[C@@H](Cc1ccc(NP(=O)(O)O)cc1)C(=O)O] | phosphoramidate (pnY) | 17-fold | Y⁶ | Y | Serwa, et al., Angew Chem Int Ed Engl 48, 8234-8239 (2009), this work |

Compared with pTyr, binding affinities of peptides with sY, pCMF, and Pmp to SH2 domains are reduced by 160-, 450-, and 6-fold, respectively (Ju et al., Mol Biosyst 9, 1829-1832 (2013)). Furthermore, these three analogs are resistant to dephosphorylation by phosphatases, and therefore do not allow probing of the full dynamics of phosphorylation at these sites. Previously, there is no established methodology to robustly and selectively produce proteins with pTyr (or a hydrolysable analog). Thus, compositions and methods of making pnY, and compositions formed therefrom are provided. Such production of such proteins can be used to, for example, facilitate the study of the dynamic role of pTyr in proteins. Exemplified in Examples 9-12 below, the data show that pnY offers a robust, scalable, site-specific, and recyclable method for the study of tyrosine phosphorylation.

The methods typically include production of pAzF-containing protein, optionally, but preferably, using a genomically recoded E. coli (Isaacs et al., Science, 333, 348-53 (2011), Lajoie et al., Science 342, 357-360 (2013)). In some embodiments, the methods also utilize an improved pAcFRS.1.t1 aaRS-tRNA pair (Amiram et al., Nat Biotechnol 33, 1272-1279 (2015)).

Optionally, but preferably, a diazotransfer reaction such as those disclosed herein, can be used to increase post-translational recovery of pAzF.

Next, a Staudinger-phosphite ligation is utilized to convert pAzF to pnY, also referred to herein as “writing” pnY. For example, in the experiments below, the protein was treated with tris(4-(2,5,8,11,14-pentaoxahexadecan-16-yloxy)-5-methoxy-2-nitrobenzyl) phosphite (TNBP, below) (100 eq., 16 h), producing Cav(pnY-protected)GFP.

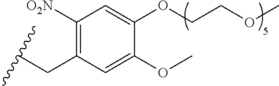

R-group of P(OR)₃, i.e. tris(4-(2,5,8,11,14-pentaoxahexadecan-16-yloxy)-5-methoxy-2-nitrobenzyl) phosphite (TNBP). The reaction can be carried out at 0, 4, 10, 20° C., preferably at 4° C. The preferred pH for the reaction is 8.0.

Next, deprotection is carried out. The Examples below show that deprotection can be carried out by laser (e.g., 355 nm, 90 sec) and sunlight (1, 2, or 3 hours). Thus, suitable means of deprotection include, but are not limited to, exposure of UV light from e.g., UV-spectrum lasers (Serwa et al., Angew Chem Int Ed Engl 48, 8234-8239 (2009)), LEDs, or other lamps, or sunlight for an effective amount of time. Strong UV sources may require less exposure time and weaker UV sources may require more exposure time, relatively speaking.

In the experiments below, the reaction was carried out in alkaline buffered aqueous conditions, compatible with the pnY-containing polypeptides and interaction partner. The reactions can be performed at 4, 10, 20, 30 or 37° C., preferably at 20° C.

The resulting pnY-containing polypeptides can be utilized for a variety of applications including testing of putative pTyr binding domains (as referred to herein as “reading”) and phosphatases (also referred to herein as “erasing”). Assays can include binding affinity testing, and competition assays which can be used to determine the EC50.

For example, in some embodiments, a method of determining affinity of a putative binding domain can include, mixing pnY-containing polypeptide with the putative binding domain or a larger protein including the domain, or titration set thereof. EC50 for percent inhibition can estimated by plotting log₁₀[agonist] vs. response. See, e.g., Jarmoskaite, et al., eLife 2020; 9:e57264 DOI: 10.7554/eLife.57264 In some embodiments, a method of determining the activity of a putative phosphatase can include, for example, mixing pnY-containing polypeptide with the putative phosphatase or titration set thereof, and assess the phosphorylation status of the pnY-containing. Methods of assessing phosphorylation status are known in the art and may include, for example, staining with phospho-antibody (e.g., Western blot), liquid chromatography (including, e.g., HPLC), mass spectroscopy, or a combination thereof. These phosphatase assays may be used to identify or characterize physiologically relevant phosphatases.

A diazotransfer reaction utilized for example, as described herein, can be used to regenerate pAzF-containing protein from the amine degradation product of both pnY and pAzF (“re-writing”). Thus, in some embodiments, the methods include treating degraded pnY and pAzF with a diazotransfer reaction.

### III. Compositions and Methods of Making Polypeptides with NSAAs

The improvements and enhancements introduced above are typically employed as an element of or subsequent to a method of making pAzF-containing polypeptides. Thus, methods of making polypeptides containing one or more instances of pAzF alone or in combination with one or more additional non-standard amino acids (nsAAs) are also provided.

Several methods of making nsAA-containing polypeptides are known in the art. A first approach introduces an nsAA by complete amino acid replacement wherein a natural amino acid is substituted for a close synthetic analog (i.e., the nsAA) in an auxotrophic strain (Dougherty, et al., Macromolecules, 26:1779-1781 (1993)).

Preferably, as discussed in more detail below, nsAAs can be incorporated via codon reassignment or frameshift codons using orthogonal translation systems (OTSs) having an aminoacyl tRNA synthetases (“AARS”) that is only able to charge a cognate tRNA, which is not aminoacylated by endogenous AARSs (Liu, et al., Annu Rev Biochem, 79:413-44 (2010), Chin, et al., Annu Rev Biochem, (2014), Amiram, et al., Nat Biotechnol., 33, 1272-1279 (2015), WO 2015/120287).

### A. Systems

Systems for making polypeptides including one or azides are provided. The systems typically include a host organism as well as an aminoacyl-tRNA synthetase (AARS) and paired transfer RNA (tRNA) pair (i.e., an orthogonal pair), and an mRNA encoding a polypeptide. The AARS, tRNA, and mRNA are typically heterologous to the host organism. In preferred embodiments, the host system is a genomically recoded organism (GRO). A GRO is an organism that has been recoded such that at least one codon is deleted from most, or preferable all, its instances in the organism's genome. The heterologous tRNA can include an anticodon that recognizes the reduced or missing codon. The heterologous AARS is one that can charge it's paired heterologous tRNA with a non-standard amino acid. When a heterologous mRNA including at least one iteration of the GRO-deleted codon is expressed in the host in the presence of the non-standard amino acid, the non-standard amino acid is incorporated into the polypeptide by the heterologous tRNA during translation of the heterologous mRNA.

### 1. Host Organisms

a. In vivo Methods

When translation is carried out in vivo using a genomically recoded organism (GRO) or other host organism, nucleic acids encoding the orthogonal AARS and tRNA operably linked to one or more expression control sequences are introduced or integrated into cells or organisms. The heterologous mRNA encoding the protein of interest is introduced or integrated into host cells or organisms, and can also be linked to an expression control sequence.

i. Genomically Recoded Organisms

The host can be a genomically recoded organism (GRO). The GRO can be transformed or genetically engineered to express the orthogonal AARS-tRNA pair and the mRNA of interest. As discussed in more detail below, the AARS-tRNA pair and mRNA of interest transformed or transfected into the host expressed extrachomasomally, for example by plasmid(s) or another vector(s) or an episome, or can be integrated into the host's genome. The GRO host organism prior to transfection or integration of the AARS-tRNA pair can be referred to as a precursor or parental GRO.

Typically, the GRO is a cell or cells, preferably a bacterial strain, for example, an E. coli bacterial strain, wherein one or more codons has been replaced by a synonymous or even a non-synonymous codon. Because there are 64 possible 3-base codons, but only 20 canonical amino acids (plus stop codons), some amino acids are coded for by 2, 3, 4, or 6 different codons (referred to herein as “synonymous codons”). In a GRO, most or preferably all, of the instances of a particular codon are replaced with a synonymous (or non-synonymous) codon. Preferably, the GRO is recoded such that at least one codon is completely absent from the genome (also referred to as an eliminated codon). In some embodiments, two, three, four, five, six, seven, eight, nine, ten, or more codons are eliminated. Removal of a codon from the precursor GRO allows reintroduction of the deleted codon in a heterologous mRNA of interest. As discussed in more detail below, the reintroduced codon is typically dedicated to a pAzF amino acid, which in the presence of the appropriate orthogonal translation machinery, can be incorporated in the nascent peptide chain of during translation of the mRNA.

When a sense codon is eliminated, its elimination is preferably accompanied by mutation, or reduction or elimination of expression, of the cognate tRNA that decodes the codon during translation, reducing or eliminating the recognition of the codon by the tRNA. For example, the tRNA can be deleted from the organism, the tRNA can be mutated to recognized fewer or different codons (e.g., from recognizing AUA and AUC to just recognizing AUC), etc. In preferred embodiments, tRNAs that decode a particular codon(s) are deleted, as in some instances (due to Wobble effect), one tRNA decodes >1 codon (e.g., AGG, AGA).

When a nonsense codon is eliminated, its elimination is preferably accompanied by mutation, reduction, or deletion of the endogenous factor or factors, for example, release factor(s), associated with terminating translation at the nonsense codon (e.g., to reduce or eliminate expression of the release factor or change the recognition specificity of codons for the release factor).

In some embodiments, wherein the organism does not have or use certain codon(s), the unused (i.e., eliminated) codon may not be strictly considered sense or nonsense codons, but can nonetheless be utilized in the strategies discussed herein. For example, a host organism can be created by taking a codon an organism does not have or use, but can still be recognized (see. e.g., Krishnakumar, et al., Chembiochem., 14(15):1967-72 (2013). doi: 10.1002/cbic.201300444) and mutating its translation machinery, e.g., tRNA and/or factors such release factors, to have a greater specificity, thus creating an unassigned codon.

In some embodiments, a sense codon is reassigned as a nonsense codon. Typically a release factor that recognizes the reassigned nonsense codon is also expressed by such organisms.

Different organisms often show particular preferences for one of the several codons that encode the same amino acid, and some codons are considered rare or infrequent. Preferably, the replaced codon is one that is rare or infrequent in the genome. The replaced codon can be one that codes for an amino acid (i.e., a sense codon) or a translation termination codon (i.e., a stop codon). GRO that are suitable for use as host or parental strains for the disclosed systems and methods are known in the art, or can be constructed using known methods. See, for example, Isaacs, et al., Science, 333, 348-53 (2011), Lajoie, et al., Science 342, 357-60 (2013), Lajoie, et al., Science, 342, 361-363 (2013). Chin, et al., Nature, 569(7757):514-518 (2019). doi: 10.1038/s41586-019-1192-5, Ostrov, et al., Science, 353(6301):819-22 (2016). doi: 10.1126/science.aaf3639. See also the Sc2.0 project focused on synthesizing a new version of Saccharomyces cerevisiae refer to as Sc2.0.

In some embodiments, the eliminated codon is one that codes for a rare stop codon. In a particular embodiment, the GRO is one in which all instances of the UAG (TAG) codon have been removed and replaced by another stop codon (e.g., TAA, TGA), and preferably wherein release factor 1 (RF1; terminates translation at UAG and UAA) has also been deleted, eliminating translational termination at UAG codons (Lajoie, et al., Science 342, 357-60 (2013)). In a particular embodiment, the GRO is C321.Δ A [321 UAG→UAA conversions and deletion of prfA (encodes RF1)](genome sequence at GenBank accession CP006698), or a further modified strain thereof. In this GRO the UAG is eliminated. That is, UAG has been transformed from a nonsense codon (terminates translation). UAG is a preferred codon for elimination or recoding because it is the rarest codon in Escherichia coli MG1655 (321 known instances) and a rich collection of translation machinery capable of incorporating non-standard amino acids has been developed for UAG (Liu and Schultz, Annu. Rev. Biochem., 79:413-44 (2010), discussed in more detail below).

Stop codons include TAG (UAG), TAA (UAA), and TGA (UGA). Although recoding to UAG (TAG) is discussed in more detail above, it will be appreciated that either of the other stop codons (or any sense codon) can be elimination and optionally reintroduced using the same strategy. Accordingly, in some embodiments, a sense codon is eliminated, e.g., AGG or AGA to CGG, CGA, CGC, or CGG (arginine), e.g., as the principles can be extended to any set of synonymous or even non-synonymous codons, that are coding or non-coding. The foregoing is non-limiting example.

Similarly, the cognate translation machinery can be removed/mutated/deleted to remove natural codon function (e.g., nonsense codons UAG—RF1; UGA−RF2; tRNA corresponding to an eliminated sense codon, etc). The OTS system, particularly the antisense codon of the tRNA, can be designed to match a reintroduced codon, provided at least one codon remains eliminated. See also, Chin, et al., Nature, 569(7757):514-518 (2019). doi: 10.1038/s41586-019-1192-5, e.g., isoleucine, and Ostrov, et al., Science, 353(6301):819-822 (2016) DOI: 10.1126/science.aaf3639, which describes reducing the number of codons in E. coli from 64 to 57 by removing instances of the UAG stop codon and excising two arginine codons, two leucine codons, and two serine codons

Prokaryotes useful as GRO cells include, but are not limited to, gram negative or gram positive organisms such as E. coli or Bacilli, and although the most preferred host organism is a bacterial GRO, it will be appreciated the methods and compositions disclosed herein can be adapted for use on other host GRO organisms, including, but not limited to, eukaryotic cells, including e.g., yeast, fungi, insect, plant, animal, human, etc. cells, and, viruses.

GRO can have two, three, or more codons replaced with a synonymous codon. Such GRO allow for reintroduction of the two, three, or more deleted codons in a heterologous mRNA of interest, each dedicated to a different non-standard amino acid. Such GRO can be used in combination with the appropriate orthogonal translation machinery to produce polypeptides having two, three, or more different non-standard amino acids.

ii. Other In Vivo Host Systems

Although the most preferred host organism is a GRO, it will be appreciated the methods and compositions disclosed herein can be adapted for use on other host organisms or in vitro. Other hosts and in vitro systems for translation are known in the art.

Suitable organisms include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems.

It will be understood by one of ordinary skill in the art that regardless of the system used (i.e. in vitro or in vivo), expression of genes encoding orthogonal AARS and tRNA will result in site specific incorporation of non-standard amino acids such as pAzF into the target polypeptides or proteins encoded by the specific heterologous mRNA transfected or integrated into the organism. Host cells are genetically engineered (e.g., transformed, transduced or transfected) with the vectors encoding orthogonal AARS, tRNA and heterologous mRNA which can be, for example, a cloning vector or an expression vector. The vector can be, for example, in the form of a plasmid, a bacterium, a virus, a naked polynucleotide, or a conjugated polynucleotide. The vectors are introduced into cells and/or microorganisms by standard methods including electroporation, infection by viral vectors, high velocity ballistic penetration by small particles with the nucleic acid either within the matrix of small beads or particles, or on the surface. Such vectors can optionally contain one or more promoter. A “promoter” as used herein is a DNA regulatory region capable of initiating transcription of a gene of interest.

Kits are commercially available for the purification of plasmids from bacteria, (see, e.g., GFX™ Micro Plasmid Prep Kit from GE Healthcare; STRATAPREP® Plasmid Miniprep Kit and STRATAPREP® EF Plasmid MIDIPREP Kit from Stratagene; GENELUTE™ HP Plasmid Midiprep and MAXIPREP Kits from Sigma-Aldrich, and, Qiagen plasmid prep kits and QIAfilter™ kits from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems.

Prokaryotes useful as host cells include, but are not limited to, gram negative or gram positive organisms such as E. coli or Bacilli. In a prokaryotic host cell, a polypeptide may include an N-terminal methionine residue to facilitate expression of the recombinant polypeptide in the prokaryotic host cell. The N-terminal Met may be cleaved from the expressed recombinant polypeptide. Promoter sequences commonly used for recombinant prokaryotic host cell expression vectors include lactamase and the lactose promoter system.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. To construct an expression vector using pBR322, an appropriate promoter and a DNA sequence are inserted into the pBR322 vector. Other commercially available vectors include, for example, T7 expression vectors from Invitrogen, pET vectors from Novagen and pALTER® vectors and PinPoint® vectors from Promega Corporation.

Yeasts useful as host cells include, but are not limited to, those from the genus Saccharomyces, Pichia, K Actinomycetes and Kluyveromyces. Yeast vectors will often contain an origin of replication sequence, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes (Holland et al., Biochem. 17:4900, (1978)) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Fleer et al., Gene, 107:285-195 (1991), in Li, et al., Lett Appl Microbiol. 40(5):347-52 (2005), Jansen, et al., Gene 344:43-51 (2005) and Daly and Hearn, J. Mol. Recognit. 18(2):119-38 (2005). Other suitable promoters and vectors for yeast and yeast transformation protocols are well known in the art.

Mammalian or insect host cell culture systems well known in the art can also be employed for producing proteins or polypeptides. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are particularly useful because both are easily obtained from a viral genome as a fragment which may also contain a viral origin of replication. Exemplary expression vectors for use in mammalian host cells are well known in the art.

b. In vitro Transcription/Translation

In some embodiments, the nucleic acids encoding AARS and tRNA are synthesized prior to translation of the target protein and are used to incorporate pAzF into a target protein in a cell-free (in vitro) protein synthesis system.

In vitro protein synthesis systems involve the use crude extracts containing all the macromolecular components (70S or 80S ribosomes, tRNAs, aminoacyl-tRNA synthetases, initiation, elongation and termination factors, etc.) required for translation of exogenous RNA. To ensure efficient translation, each extract must be supplemented with amino acids, energy sources (ATP, GTP), energy regenerating systems (creatine phosphate and creatine phosphokinase for eukaryotic systems, and phosphoenol pyruvate and pyruvate kinase for the E. coli lysate), and other co-factors (Mg2+, K+, etc.).

In vitro protein synthesis does not depend on having a polyadenylated RNA, but if having a poly(A) tail is essential for some other purpose, a vector may be used that has a stretch of about 100 A residues incorporated into the polylinker region. That way, the poly(A) tail is “built in” by the synthetic method. In addition, eukaryotic ribosomes read RNAs that have a 5′ methyl guanosine cap more efficiently. RNA caps can be incorporated by initiation of transcription using a capped base analogue, or adding a cap in a separate in vitro reaction post-transcriptionally.

Suitable in vitro transcription/translation systems include, but are not limited to, the rabbit reticulocyte system, the E. coli S-30 transcription-translation system, the wheat germ based translational system. Combined transcription/translation systems are available, in which both phage RNA polymerases (such as T7 or SP6) and eukaryotic ribosomes are present. One example of a kit is the TNT® system from Promega Corporation.

### 2. Orthogonal Translation System

Translation systems include most or all of the translation machinery of the host organism and additionally include a heterologous aminoacyl-tRNA synthetase (AARS)-rRNA pair (also referred to as an orthogonal translation system (OTS)) that can incorporate one or more pAzF into a growing peptide during translation of the heterologous mRNA. AARS are enzymes that catalyze the esterification of a specific cognate amino acid or its precursor to one or all of its compatible cognate tRNAs to form an aminoacyl-tRNA. An AARS can be specific for pAzF, or can be polyspecific for two or more non-standard amino acids, canonical amino acids, or a combination thereof. The heterologous AARS used in the disclosed system typical can recognize, bind to, and transfer at least one non-standard amino acid to a cognate tRNA. Accordingly, the AARS can be selected by the practitioner based on the non-standard amino acid on interest.

Some of the disclosed systems include two or more heterologous AARS. tRNA is an adaptor molecule composed of RNA, typically about 76 to about 90 nucleotides in length that carries an amino acid to the protein synthetic machinery. Typically, each type of tRNA molecule can be attached to only one type of amino acid, so each organism has many types of tRNA (in fact, because the genetic code contains multiple codons that specify the same amino acid, there are many tRNA molecules bearing different anticodons which also carry the same amino acid). The heterologous tRNA used in the disclosed systems is one that can bind to the selected heterologous AARS and receive a non-standard amino acid to form an aminoacyl-tRNA. Because the transfer for the amino acid to the tRNA is dependent in-part on the binding of the tRNA to the AARS, these two components are typically selected by the practitioner based on their ability to interact with each other and participate in protein synthesis including the non-standard amino acid of choice in the host organism. Therefore, a selected heterologous AARS and tRNA are often referred to herein together as a heterologous AARS-tRNA pair, or an orthogonal translation system. Preferably, the heterologous AARS-tRNA pair does not cross-react with the existing host cell's pool of synthetases and tRNAs, or do so a low level (e.g., inefficiently), but is recognized by the host ribosome. Therefore, preferably the heterologous AARS cannot charge an endogenous tRNA with a non-standard amino acid (or does so a low frequency), and/or an endogenous AARS cannot charge the heterologous tRNA with a standard amino acid.

Furthermore, preferably, the heterologous AARS cannot charge its paired heterologous tRNA with a standard amino acid (or does so at low frequency).

The heterologous tRNA also includes an anticodon that recognizes the codon of the codon in the heterologous mRNA that encodes the non-standard amino acid of choice. In the most preferred embodiment, the anticodon is one that hybridizes with a codon that is reduced or deleted in the host organism and reintroduced by the heterologous mRNA. For example, if the reduced or deleted codon is UAG (TAG), as in C321.Δ A, the heterologous tRNA anticodon is typically CUA.

In the disclosed system at least one orthogonal pair is dedicated to incorporation of pAzF into a polypeptide. However, the system may include two, three, or more orthogonal pairs, where one is dedicated to pAzF and one or more are dedicated to incorporation of one or more other non-standard amino acids.

The AARS-tRNA pair can be from an achaea, such as Methanococcus maripaludis, Methanocaldococcus jannaschii, Methanopyrus kandleri, Methanococcoides burtonii, Methanospirillum hungatei, Methanocorpusculum labreanum, Methanoregula boonei, Methanococcus aeolicus, Methanococcus vannieli, Methanosarcina mazei, Methanosarcina barkeri, Methanosarcina acetivorans, Methanosaeta thermophila, Methanoculleus marisnigri, Methanocaldococcus vulcanius, Methanocaldococcus fervens, or Methanosphaerula palustris, for can be variant evolved therefrom.

Suitable heterologous AARS-tRNA pairs for use in the disclosed systems and methods are known in the art. For example, Table 1 and the electronic supplementary information provided in Dumas, et al., Chem. Sci., 6:50-69 (2015), provide non-natural amino acids that have been genetically encoded into proteins, the reported mutations in the AARS that permit their binding to the non-natural amino acid, the corresponding tRNA, and a host organism in which the translation system is operational. See also Liu and Schultz, Annu. Rev. Biochem., 79:413-44 (2010) and Davis and Chin, Nat. Rev. Mol. Cell Biol., 13:168-82 (2012), which provide additional examples of AARS-tRNA pairs which can be used in the disclosed systems and methods. Preferred AARS with improved activity and specificity for the specific non-naturally occurring amino acids are disclosed and described in WO 2015/120287, which is specifically incorporated by reference herein in its entirety.

The AARS and tRNA can be provided separately, or together, for example, as part of a single construct. In a particular embodiment, the AARS-tRNA pair is evolved from a Methanocaldococcus jannaschii aminoacyl-tRNA synthetase(s) (AARS)/suppressor tRNA pairs and suitable for use in an E. coli host organism. See, for example, Young, J. Mol. Biol., 395(2):361-74 (2010), which describes an OTS including constitutive and inducible promoters driving the transcription of two copies of a M. jannaschii AARS gene in combination with a suppressor tRNA(CUA)(opt) in a single-vector construct.

During protein synthesis, tRNAs with attached amino acids are delivered to the ribosome by proteins called elongation factors (EF-Tu in bacteria, eEF-1 in eukaryotes), which aid in decoding the mRNA codon sequence. If the tRNA's anticodon matches the mRNA, another tRNA already bound to the ribosome transfers the growing polypeptide chain from its 3′ end to the amino acid attached to the 3′ end of the newly delivered tRNA, a reaction catalyzed by the ribosome. Accordingly, the heterologous AARS-tRNA pair should be one that can be processed by the host organism's elongation factor(s). Additional or alternatively, the system can include additional or alternative elongation factor variants or mutants that facilitate delivery of the heterologous aminoacyl-tRNA to the ribosome.

It will also be appreciated that methods of altering the anticodon of tRNA are known in the art. Any suitable tRNA selected for use in the disclosed systems and methods can be modified to hybridize to any desired codon. For example, although many of the heterologous tRNA disclosed here and elsewhere have a CUA anticodon, CUA can be substituted for another stop anticodon (e.g., UUA or UCA), or anticodon for any desired sense codon. The tRNA anticodon can be selected based on the GRO and the sequence of the heterologous mRNA as discussed in more detail above.

The OTS can also include mutated EF-Tu, in addition to AARS and tRNA, especially for bulky and/or highly charged NSAAs (e.g., phosphorylated amino acids) (Park, et al., Science, 333:1151-4 (2011)).

### B. Methods Making Polypeptides

The methods typically involve using an orthogonal AARS-tRNA pair in the translation process for a target polypeptide from heterologous mRNA of interest. As discussed above, the AARS preferentially aminoacylates its cognate tRNA with a non-naturally occurring amino acid such pAzF. The resulting aminoacyl-tRNA recognizes at least one codon in the mRNA for the target protein, such as a stop codon. An elongation factor (such as EF-Tu in bacteria) mediates the entry of the amninoacyl-tRNA into a free site of the ribosome. If the codon-anticodon pairing is correct, the elongation factor hydrolyzes guanosine triphosphate (GTP) into guanosine diphosphate (GDP) and inorganic phosphate, and changes in conformation to dissociate from the tRNA molecule. The aminoacyl-tRNA then fully enters the A site, where its non-standard amino acid is brought near the P site's polypeptide and the ribosome catalyzes the covalent transfer of the pAzF onto the polypeptide.

In some embodiments, as discussed above, the resulting polypeptides are treated with diazotransfer reaction, modified to include a further moiety or moieties using e.g., copper(I)-catalyzed azide-alkyne Huisgen cycloaddition (click-chemistry).

The resulting polypeptides can be isolated, purified, or otherwise enriched using methods known in the art, and discussed in more detail below.

In some embodiments, the heterologous AARS, its cognate tRNA, or more preferably both, are integrated into the host genome. Although suitable AARS are known in the art, in the most preferred embodiments, the AARS is a variant AARS that has improved binding to its cognate tRNA, its non-standard amino acid(s), or both compared to a known AARS. Exemplary variant AARS are discussed in more detail below.

The methods of making polypeptide are typically capable of producing polypeptides having a greater number of instances of non-standard amino acids and/or a greater yield of the desired polypeptide than the same or similar polypeptide made using conventional compositions, systems, and methods.

C. Compositions for Making Polypeptides with Nonstandard Amino Acids

### 1. Variant AARS

Methods of making AARS are provided in WO 2015/120287, and variant AARS obtaining according to the method, including, but not limited to those provided in WO 2015/120287 are provided and can be used in the disclosed methods. DNA sequence(s) can also be deduced from the amino acid sequence of the variant. Accordingly, nucleic acid sequences encoding variant AARS are also provided.

The precise percentage of similarity between sequences that is useful in establishing sequence identity varies with the nucleic acid and protein at issue, but as little as 25% sequence similarity is routinely used to establish sequence identity. Higher levels of sequence similarity, e.g., at least 30%, 40%, 50%, 60%, 70%, 80%, 90%, 95% or 99% or more can also be used to establish sequence identity. Therefore, in some embodiments, the variant includes at least 30%, 40%, 50%, 60%, 70%, 80%, 90%, 95% or 99% or more sequence identity with the parent AARS.

Variant AARS of a parent M. jannaschii AARS referred to pAcF AARS (pAcFRS) (Young, et al., J Mol Biol, 395:361-74 (2010)) are provided. The amino acid sequence for pAcFRS is

(SEQ ID NO: 1)
MDEFEMIKRNTSEIISEEELREVLKKDEKSALIGFEPSGKIHLGHYLQIKK
MIDLQNAGEDIIILLADLHAYLNQKGELDEIRKIGDYNKKVFEAMGLKAKY
VYGSEFQLDKDYTLNVYRLALKTTLKRARRSMELIAREDENPKVAEVIYPI
MQVNGCHYRGVDVAVGGMEQRKIHMLARELLPKKVVCIHNPVLTGLDGEGK
MSSSKGNFIAVDDSPEEIRAKIKKAYCPAGVVEGNPIMEIAKYFLEYPLTI
KRPEKFGGDLTVNSYEELESLFKNKELHPMRLKNAVAEELIKILEPIRKR
L.

The nucleic acid sequence for a cognate tRNA of SEQ ID NO:1 is CCGGCGGTAGTTCAGCAGGGCAGAACGGCGGACTCTAAATCCGCATGGCAG GGGTTCAAATCCCCTCCGCCGGACCA SEQ ID NO:17. This tRNA can also be a congnate tRNA for the variant AARS described in more detail below.

Variants of pAcFRS have one or more mutations relative to SEQ ID NO:1, and typically have altered specificity and/or activity toward one or more non-standard amino acids and/or altered specificity and/or activity toward a paired tRNA relative to the protein of SEQ ID NO:1. In some embodiments, the variant includes at least 30%, 40%, 50%, 60%, 70%, 80%, 90%, 95% or 99% or more sequence identity with the parent AARS, or a functional fragment thereof.

The variants typically have one or more substitution mutations in the non-standard amino acid (amino acid ligand) binding pocket of SEQ ID NO:1, the tRNA anticodon recognition interface of SEQ ID NO:1, or a combination thereof. For example, the variants can have a substitution mutation at one or more of amino acid positions 65, 107, 108, 109, 158, 159, 162, 167, 257, and 261 of SEQ ID NO:1 relative to the N-terminal methionine of SEQ ID NO:1.

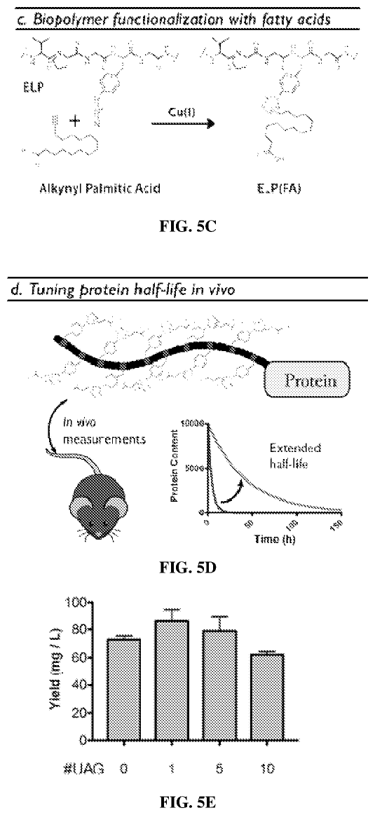

Exemplary variants are provided below and have nsAA specificities at least as provided. The relative polyspecificities (or monospecificy) of each are discussed in more detail in the working Examples and FIGS. 5A-5M. pAcFRS.1 (polyspecifity for at least pAcF, pAzF, StyA, 4IF, 4BrF, 4ClF, 4MeF, 4Cf3F, MeY, 4NO2F, 4BuF, BuY, 2NaA, PheF):

(SEQ ID NO: 2)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSEFQLDKDYTLNVYRLALKTTLKRARRSME
LIAREDENPKVAEVIYPIMQVNGCHYRGVDVDVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKRPEKFGGDLTVNSY
EELESLFKNKELHPMRLKNAVAEELIKILEPIRKRL;
pAcFRS.t1 (polyspecifity for at least pAcF,
pAzF, StyA):
(SEQ ID NO: 3)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSEFQLDKDYTLNVYRLALKTTLKRARRSME
LIAREDENPKVAEVIYPIMQVNGCHYRGVDVAVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKGPEKFGGDLTVNSY
EELESLFKNKELHPMRLKNAVAEELIKILEPIRKRL;
pAcFRS.t2 (polyspecifity for at least pAcF,
pAzF, StyA):
(SEQ ID NO: 4)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSEFQLDKDYTLNVYRLALKTTLKRARRSME
LIAREDENPKVAEVIYPIMQVNGCHYRGVDVAVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKCPEKEGGDLTVNSY
EELESLFKNKELHPMRLKNAVAEELIKILEPIRKRL;
pAcFRS.1.t1 (polyspecifity for at least pAcF,
pAzF, StyA, 4IF, 4BrF, 4CIF, 4MeF, 4Cf3F,
MeY, 4NO2F, 4BuF, BuY, 2NaA, PheF):
(SEQ ID NO: 5)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSEFQLDKDYTLNVYRLALKTTLKRARRSME
LIAREDENPKVAEVIYPIMQVNGCHYRGVDVDVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKGPEKFGGDLTVNSY
EELESLFKNKELHPMRLKNAVAEELIKILEPIRKRL;
pAcFRS.1.t2 (polyspecifity for at least pAcF,
pAzF, StyA, 4IF, 4BrF, 4CIF, 4MeF, 4Cf3F,
MeY, 4NO2F, 4BuF, BuY, 2NaA, PheF):
(SEQ ID NO: 6)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSEFQLDKDYTLNVYRLALKTTLKRARRSME
LIAREDENPKVAEVIYPIMQVNGCHYRGVDVDVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKCPEKEGGDLTVNSY
EELESLFKNKELHPMRLKNAVAEELIKILEPIRKRL;
pAcFRS.2 (polyspecifity for at least pAcF,
pAzF, StyA, 4IF, 4BrF, 4CIF, 4MeF, 4Cf3F,
MeY, 4NO2F, 4BuF, BuY, 2NaA, PheF).
(SEQ ID NO: 7)
MDEFEMIKRNTSEIISEEELREVLKKDEKSALIGFEPSGKIHLGH
YLQIKKMIDLQNAGFDIIIVLADLHAYLNQKGELDEIRKIGDYNK
KVFEAMGLKAKYVYGSEFQLDKDYTLNVYRLALKTTLKRARRSME
LIAREDENPKVAEVIYPIMQVNGCHYRGVDVDVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKRPEKFGGDLTVNSY
EELESLFKNKELHPMRLKNAVAEELIKILEPIRKRL;
pAcFRS.2.t1 (polyspecifity for at least pAcF,
pAzF, StyA, 4IF, 4BrF, 4CIF, 4MeF, 4Cf3F,
MeY, 4NO2F, 4BuF, BuY, 2NaA, PheF)
(SEQ ID NO: 8)
MDEFEMIKRNTSEIISEEELREVLKKDEKSALIGFEPSGKIHLGH
YLQIKKMIDLQNAGFDIIIVLADLHAYLNQKGELDEIRKIGDYNK
KVFEAMGLKAKYVYGSEFQLDKDYTLNVYRLALKTTLKRARRSME
LIAREDENPKVAEVIYPIMQVNGCHYRGVDVDVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKGPEKFGGDLTVNSY
EELESLFKNKELHPMRLKNAVAEELIKILEPIRKRL;
pAcFRS.2.t2 (polyspecifity for at least pAcF,
pAzF, StyA, 4IF, 4BrF, 4CIF, 4MeF, 4Cf3F,
MeY, 4NO2F, 4BuF, BuY, 2NaAPheF):
(SEQ ID NO: 9)
MDEFEMIKRNTSEIISEEELREVLKKDEKSALIGFEPSGKIHLGH
YLQIKKMIDLQNAGFDIIIVLADLHAYLNQKGELDEIRKIGDYNK
KVFEAMGLKAKYVYGSEFQLDKDYTLNVYRLALKTTLKRARRSME
LIAREDENPKVAEVIYPIMQVNGCHYRGVDVDVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKCPEKEGGDLTVNSY
EELESLFKNKELHPMRLKNAVAEELIKILEPIRKRL;
pAzFRS.1 (specific for pAzF):
(SEQ ID NO: 10)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSEFQLDKDYTLNVYRLALKTTLKRARRSME
LIAREDENPKVAEVIYPIMQVNVMHYDGVDVYVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKRPEKFGGDLTVNSY
EELESLFKNKELHPMRLKNAVAEELIKILEPIRKRL;
pAzFRS.1.t1 (specific for pAzF):
(SEQ ID NO: 11)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSEFQLDKDYTLNVYRLALKTTLKRARRSME
LIAREDENPKVAEVIYPIMQVNVMHYDGVDVYVGGMEQRKIHMLA
RELLPKKVVCIHNPVLTGLDGEGKMSSSKGNFIAVDDSPEEIRAK
IKKAYCPAGVVEGNPIMMEIAKYFLEYPLTIKGPEKFGGDLTVNS
YEELESLFKNKELHPMRLKNAVAEELIKILEPIRKRL;
pAzFRS.1.t2 (specific for pAzF):
(SEQ ID NO: 12)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSEFQLDKDYTLNVYRLALKTTLKRARRSME
LIAREDENPKVAEVIYPIMQVNVMHYDGVDVYVGGMEQRKIHMLA
RELLPKKVVCIHNPVLTGLDGEGKMSSSKGNFIAVDDSPEEIRAK
IKKAYCPAGVVEGNPIMMEIAKYFLEYPLTIKCPEKEGGDLTVNS
YEELESLFKNKELHPMRLKNAVAEELIKILEPIRKRL;
pAzRS.2 (polyspecific for at least pAcF, pAzF,
StyA, 4IF, 4BrF, 4CIF, 4MeF, 4Cf3F, MeY, 4NO2F,
4BuF, BuY, 2NaA, PheF):
(SEQ ID NO: 13)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSTYMLDKDYTLNVYRLALKTTLKRARRSME
LIAREDENPKVAEVIYPIMQVNGCHYRGVDVAVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKRPEKFGGDLTVNSY
EELESLFKNKELHPMRLKNAVAEELIKILEPIRKRL;
pAzRS.2.t1(polyspecific for at least pAcF, pAzF,
StyA, 4IF, 4BrF, 4CIF, 4MeF, 4Cf3F, MeY, 4NO2F,
4BuF, BuY, 2NaA, PheF):
(SEQ ID NO: 14)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSTYMLDKDYTLNVYRLALKTTLKRARRSME
LIAREDENPKVAEVIYPIMQVNGCHYRGVDVAVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKGPEKFGGDLTVNSY
EELESLFKNKELHPMRLKNAVAEELIKILEPIRKRL;
and
pAzRS.2.t2 (polyspecific for at least pAcF, pAzF,
StyA, 4IF, 4BrF, 4CIF, 4MeF, 4Cf3F, MeY, 4NO2F,
4BuF, BuY, 2NaA, PheF):
(SEQ ID NO: 15)
MDEFEMIKRNTSEIISEEELREVLKKDEKSALIGFEPSGKIHLGH
YLQIKKMIDLQNAGEDIIILLADLHAYLNQKGELDEIRKIGDYNK
KVFEAMGLKAKYVYGSTYMLDKDYTLNVYRLALKTTLKRARRSME
LIAREDENPKVAEVIYPIMQVNGCHYRGVDVAVGGMEQRKIHMLA
RELLPKKVVCIHNPVLTGLDGEGKMSSSKGNFIAVDDSPEEIRAK
IKKAYCPAGVVEGNPIMEIAKYFLEYPLTIKCPEKEGGDLTVNSY
EELESLFKNKELHPMRLKNAVAEELIKILEPIRKRL.

The position and domain of the mutation in each of SEQ ID NO:2-15 relative to SEQ ID NO:1 is provided in Table 2 below. Variants having any combination of the mutations disclosed in Table 2 are also specifically provided.

TABLE 2
Annotations of specific mutations in AARS variants
(mutations in evolved synthetases are annotated
with respect to the progenitor pAcFRS variant)

| | | Evolved for |
|---|---|---|
| Annotation | Mutant | nsAA/tRNA |
| pAcFRS | pAcFRS (Young, et al., J Mol | — |
| | Biol, 395: 361-74 (2010)) | |
| pAzFRS | pAzFRS (Schultz, et al., J Am | — |
| | Chem Soc, 128: 13984-5 (2006)) | |
| pAcFRS.1 | A167D | pAcF |
| pAcFRS.t1 | R257G | tRNA |
| pAcFRS.t2 | R257C, F261E | tRNA |
| pAcFRS.1.t1 | A167D, R257G | pAcF + tRNA |
| pAcFRS.1.t2 | A167D, R257C, F261E | pAcF + tRNA |
| pAzFRS.1 | D158V, I159M, L162D, A167Y | pAzF |
| pAzFRS.1.t1 | D158V, I159M, L162D, A167Y, | pAzF + tRNA |
| | R257G | |
| pAzFRS.1.t2 | D158V, I159M, L162D, A167Y, | pAzF + tRNA |
| | R257C, F261E | |
| pAzFRS.2 | E107T, F108Y, Q109M | pAzF |
| pAzFRS.2.t1 | E107T, F108Y, Q109M, R257G | pAzF + tRNA |
| pAzFRS.2.t2 | E107T, F108Y, Q109M, R257C, | pAzF + tRNA |
| | F261E | |
| pAcFRS.2 | L65V, A167D | pAcF |
| pAcFRS.2.t1 | L65V, A167D, R257G | pAcF + tRNA |
| pAcFRS.2.t2 | L65V, A167D, R257C, F261E | pAcF + tRNA |

In some embodiments, the variant is a polypeptide including the amino acids of the non-standard amino acid (amino acid ligand) binding pocket of any of SEQ ID NO:1-15; a polypeptide including the amino acids of the tRNA anticodon recognition interface of any of SEQ ID NO:1-15; or a polypeptide including the non-standard amino acid (amino acid ligand) binding pocket and the amino acids of the tRNA anticodon recognition interface of any of SEQ ID NO:1-15. In some embodiments, the variant is a polypeptide including amino acids 65-261 of any of SEQ ID NO:1-15. All of SEQ ID NOS:1-15 are also specifically provided both with and without the N-terminal methionine. Variants having at least 30%, 40%, 50%, 60%, 70%, 80%, 90%, 95% or 99% or more sequence identity with any of SEQ ID NOS: 1-15, with and without the N-terminal methionine, and functional fragments thereof are also provided.

### D. Nucleic Acids

Polynucleotides encoding each of the proteins of SEQ ID NO:1-15, and variants and fragments thereof are also disclosed. The polynucleotides can be isolated nucleic acids, incorporated into in a vector, or part of a host genome. The polynucleotides can also be part of a cassette including nucleic acids encoding other translational components such as a paired tRNA, selection marker, promoter and/or enhancer elements, integration sequences (e.g., homology arms), etc.

### 1. Promoters and Enhancers

Nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Therefore, polynucleotides encoding each of the proteins of SEQ ID NO:1-15 operably linked to an expression control sequence are also provided

Suitable promoters are generally obtained from viral genomes (e.g., polyoma, Simian Virus 40 (SV40), adenovirus, retroviruses, hepatitis-B virus, and cytomegalovirus) or heterologous mammalian genes (e.g. beta actin promoter). Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers also often contain response elements that mediate the regulation of transcription. Many enhancer sequences are now known from mammalian genes (globin, elastase, albumin, α-fetoprotein and insulin). However, enhancer from a eukaryotic cell virus are preferably used for general expression. Suitable examples include the SV40 enhancer on the late side of the replication origin, the cytomegalovirus early promoter enhancer, the polyoma enhancer on the late side of the replication origin, and adenovirus enhancers.

In certain embodiments the promoter and/or enhancer region can act as a constitutive promoter and/or enhancer to maximize expression of the region of the transcription unit to be transcribed. In certain constructs the promoter and/or enhancer region is active in all eukaryotic cell types, even if it is only expressed in a particular type of cell at a particular time. A preferred promoter of this type is the CMV promoter. In other embodiments, the promoter and/or enhancer is tissue or cell specific.

In certain embodiments the promoter and/or enhancer region is inducible. Induction can occur, e.g., as the result of a physiologic response, a response to outside signals, or as the result of artificial manipulation. Such promoters are well known to those of skill in the art. For example, in some embodiments, the promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

### 2. Host Organisms

Host organisms whose genome is engineered to include a polynucleotide encoding any of SEQ ID NO:1-15, or a functional fragment thereof, are also provided. In a particularly preferred embodiment, the host organism is a GRO. Accordingly, genetically recoded organisms wherein a heterologous AARS, a heterologous tRNA, or a combination thereof is incorporated in the organism's genome are also provided. In some embodiments, the organism's genome includes a nucleic acid sequence encoding the AARS variant of any one of SEQ ID NO:1-15, or a functional fragment or variant thereof. The GRO can be bacteria, for example E. coli. In a particular embodiment, the E. coli is C321.ΔA.

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of delivery, such as a liposome, so that the nucleic acid contained in the delivery system can become integrated into the host genome. Techniques for integration of genetic material into a host genome are also known and include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

An exemplary orthogonal translation system integration cassette can include homology arms as well as nucleic acids sequences encoding an AARS and its cognate tRNA each operably linked to a promoter.

### IV. Polypeptides, Peptide Compositions, and Methods of Use Thereof

### A. Polypeptides

Polypeptides including one or more instances of one or more different non-standard amino acids are also provided. In preferred embodiments, the polypeptides are prepared using one or more of the variant AARS provided herein, and/or according to the methods of making polypeptides including non-standard amino acids provided herein. The polypeptides typically have one or more pAzF residues. The pAzF residues can be modified or have one or more additional moieties conjugated thereto, e.g., by click-chemistry, or other chemical reaction. For example, in some embodiments, the pAzF residue(s) are modified to include e.g. a fatty acid moiety. In other embodiments, the pAzF residue(s) are converted to pnY e.g., a Staudinger-phosphite ligation.

The polypeptide can have any sequence dictated by the practitioner. As discussed herein, the practitioner can design a heterologous mRNA encoding the polypeptide can designed using a recoded codon (e.g., a stop codon such as UAG) to encode the non-standard amino acid. When the mRNA is expressed in a translation system in the presence of the non-standard amino acid (e.g., pAzF), and the translation system includes an AARS that can aminoacylate a cognate tRNA having an anticodon that recognizes the recoded codon with the non-standard amino, the non-standard amino acid will be incorporated into the nascent peptide during translation of the mRNA.

The polypeptides can be monomeric or polymeric. A monomer is a molecule capable of reacting with identical or different molecules to form a polymer. Therefore, in some embodiments, the heterologous mRNA encodes a single subunit that can be part of a larger homomeric or heteromeric macromolecule. The compositions and methods can be used to produce sequence-defined polymers. In other embodiments, the mRNA encodes two or more subunits, for example, two or more repeats of a monomer. In some embodiments, the mRNA encodes a fusion protein including a sequence having at least one non-standard amino acid (e.g., pAzF) fused to a sequence of another protein of interest. Accordingly, the polypeptide including one or more non-standard amino acids can be part of a tag or a domain of a larger multiunit polypeptide. The polypeptide can include both standard and non-standard amino acids (e.g., pAzF). In some embodiments, the biomolecule consists of a run of consecutive non-standard amino acids, (e.g., 2, 3, 4, 5, 6, 7, 8, 9, 10, or more), or consists entirely of non-standard amino acids. All instances of non-standard amino acids can be the same, or the biomolecule can include combinations of two, three, four, or more non-standard amino acids. For example, the compositions can be used to create higher order combinations of monomers to create block polymers with more diverse chemistries.

The polypeptide can have any integer “n” from 1 to 500 of any non-standard amino acid. In some embodiments, “n” is more than 500. The compositions and methods allow for template-based biosynthesis of polymers of, in principle, any length including multiple instances of nonstandard amino acids. Polypeptides made using the disclosed variant AARS and/or methods exhibit higher yields and/or higher purities when compared to the same polypeptide produced by conventional translation-based methods and synthetic chemical methods.

In addition to pAzF (or a reacted or conjugated derivative thereof), the polypeptides can have any one or more additional non-standard amino acids. Exemplary non-standard amino acids that can be incorporated into the polypeptides disclosed herein are listed in Table 11 of WO 2015/120287. The non-standard amino acid or non-standard amino acid(s) are typically selected by the practitioner based on the side chain and the desired properties and/or use of the polypeptide as discussed in more detail below.

### B. Methods of Using Polypeptides Including Non-Standard Amino Acids

Polypeptides engineered to include one or more instances of one or more pAzF alone or in combination with one or more additional non-standard amino acids have far reaching uses. Over 100 non-standard amino acids have been described containing diverse chemical groups, including post-translational modifications, photocaged amino acids, bioorthogonal reactive groups, and spectroscopic labels (Liu, et al., Annu Rev Biochem, 79:413-44 (2010); Johnson, et al., Curr Opin Chem Biol, 14:774-80 (2010), O'Donoghue, et al., Nat Chem Biol, 9:594-8 (2013), Chin, et al., Annu Rev Biochem, (2014), Seitchik, et al., J Am Chem Soc, 134:2898-901 (2012), Davis and Chin, Nature Reviews, 13:168-182 (2012)). The use of the polypeptide is typically based on the nature of the polypeptide and the specific non-standard amino acid incorporated therein. Templates for polypeptides and methods of use thereof are known in the art. For example, site-specific incorporation of a non-standard amino acid at a single position facilitates engineering of protein-drug conjugates (Tian, et al., Proc Natl Acad Sci USA, 111:1766-71 (2014)), cross-linking proteins (Furman, et al., J Am Chem Soc, 136:8411-7 (2014)), and enzymes with altered or improved function (Kang, et al., Chembiochem, 15:822-5 (2014), Wang, et al., Angew Chem Int Ed Engl, 51:10132-5 (2012)). Multi-site non-standard amino acid incorporation can further expand the function and properties of proteins and biomaterials by enabling synthesis of polypeptide polymers with programmable combinations of natural and non-standard amino acids.

The disclosed compositions and methods allow for site-specific non-standard amino acid incorporation where multiple identical non-standard amino acids provide the dominant physical and biophysical properties to biopolymers, proteins and peptides. Multi-site non-standard amino acid incorporation also facilitate design and production of post-translationally modified proteins (e.g., kinases) for the study and treatment of disease or of new biologics (e.g., antibodies) with multiple instances of new chemical functionalities.

Other biomolecules include, but are not limited to, tunable materials, nanostructures, polypeptide-based therapeutics with new properties, industrial enzymes with new chemistries and properties, bio-sensors, drug delivery vehicles, adhesives, stimuli (e.g., metals-responsvie materials), antimicrobials, synthetic peptides with enhanced pharmacokinetic properties, and biologics.

### C. Exemplary Polypeptides

### 1. Elastin-like Proteins (ELPs)

ELPs are biopolymers composed of the pentapeptide repeat Val-Pro-Gly-Xaa-Gly (VPGXG) (SEQ ID NO:17), wherein “X” can be any standard or non-standard amino acid. ELPs are discussed in U.S. Pat. No. 6,852,834, which is specifically incorporated by reference herein in its entirety, and Tang, et al., Angew Chem Int Ed Engl, 40:1494-1496 (2001), Kothakota, Journal of the American Chemical Society, 117:536-537 (1995), and Wu, Chembiochem 14:968-78 (2013). They are monodisperse, stimuli-responsive, and biocompatible, making them attractive for applications like drug delivery and tissue engineering. Moreover, ELP properties can be precisely defined and genetically encoded, making them ideal candidates for expanded function via incorporation of multiple non-standard amino acids.

Accordingly, ELPs having or including the sequence (VPGXG)ₙ (SEQ ID NO:18), wherein “X” is pAzF, and wherein “n” is an integer from 1 to 500, or more than 500 are disclosed.

Also disclosed are ELPs having or including the sequence VPGGGVPGAGVPG(X)G)_{y}(VPGGGVPGAGVPGYG)_{z} (SEQ ID NO:19) wherein “X” is pAzF, and wherein “y” is an integer from 1 to 500, or more than 500, and “z” is zero or an integer from 1 to 500, or more than 500.

In some embodiments, “n”, “y”, and/or “z” is at least 5, at least 10, at least 15, at least 20, at least 25, at least 30, at least 35, at least 40, at least 45, or at least 50. In some embodiments, “n”, “y” and/or “z” is not more than 500, not more than 250, not more than 200, not more than 100, not more than 50, not more than 45, not more than 40, not more than 35, not more than 30, not more than 25, not more than 20, not more than 15, not more than 10, or not more than 5.

The ELPs can also be a fusion protein including one or more ELP domains fused to a one or more heterologous protein. The ELP and fusion proteins can include, for example, a leader sequence, linkers between the domains, or a combination thereof.

An exemplary leader sequence is MSKGPG (SEQ ID NO:20).

An exemplary linker is PGGGG (SEQ ID NO:21).

ELP fusion proteins are exemplified below by fusion of an ELP polymer to GFP.

An exemplary GFP sequence is

(SEQ ID NO: 22)
SKGEELFTGVVPILVELDGDVNGHKFSVRGEGEGDATNGKLTLKFIC
TTGKLPVPWPTLVTTLTYGVQCFSRYPDHMKRHDFFKSAMPEGYVQE
RTISFKDDGTYKTRAEVKFEGDTLVNRIELKGIDFKEDGNILGHKLE
YNFNSHNVYITADKQKNGIKANFKIRHNVEDGSVQLADHYQQNTPIG
DGPVLLPDNHYLSTQSVLSKDPNEKRDHMVLLEFVTAAGITHGMDEL
YKGS

The ELPs can be made using the variant AARS disclosed herein and/or according to the methods of making polypeptides including non-standard amino acids disclosed herein. The ELPs disclosed herein can have more instances of a non-standard amino acid, a higher purity (e.g., reduced heterogeneity), and/or a higher yield than ELPs made according to conventional methods.

### 2. Exemplary Methods of Using ELPs

As with other polypeptides including one or more non-standard amino acids, uses for ELP include a wide range of medical and non-medical applications. The disclosed compositions and methods can be used to incorporate one or more pAzF, e.g., 1, 3, 5, 10, 15, 20, 25, 50, or more into protein polymers. Since ELPs undergo a sharp soluble-to-insoluble phase transition at their transition temperature (Tt), which depends on the ELP composition, ELP templates used for non-standard amino acid incorporation can be utilized as a scaffold for the design of smart biomaterials in which non-standard amino acid functionality can be translated to, for example, stimuli-responsiveness to light, electro-magnetic field, and various analytes. Multi-site nsAA incorporation into these and other protein-based biomaterials at high purity can modify and expand their chemical or physical properties to generate new materials.

Specific uses of ELPs are exemplified in the working Examples below.

Polypeptides including multiple instances of pAzF are also provided. The azide group of pAzF allows for the highly efficient copper-catalyzed azide-alkyne cycloaddition (“click”) chemistry reaction with alkyne containing molecules. Other reactions that can be utilized include, but are not limited to, strain promoted azide-alkyne cycloaddition, and Staudinger ligation photocrosslinking The pAzF-containing polypeptides can be functionalized with additional molecules by click addition using known methods. Suitable molecules include, but are not limited to, small molecules, proteins, etc. In some embodiments, the molecule is an active agent such a small molecule drug, and imaging agent, etc. In some embodiments, the molecule is a molecular linker that links the polypeptide to another molecule. The molecule can be any molecule with an alkyne capable of underdoing a click reaction with pAzF. The molecule can be a biomolecule.

In particular Examples below, polymers containing multiple instances of p-azidophenylalanine (pAzF) amino acid were prepared. In one Example, a fluorophore (Cy5.5) was conjugated to the pAzF creating a molecule with a detectable signal for imaging in vitro and in vivo.

In yet another example, click chemistry was used to conjugate palmitic acid-alkyne to azide group of pAzF ELPs. The resulting molecule improved serum albumin (human and mouse) binding. Fatty acid conjugation to small molecules and peptides improves in vivo pharmacokinetics profile via albumin binding. Therefore, ELPs containing pAzF to conjugate multiple fatty acid molecules per protein can be used as a platform to further enhance albumin binding and facilitate tunable enhancement (as a function of the number of fatty acid molecules) of pharmacokinetics for therapeutic proteins in vivo.

Benefits of half-life extended therapeutics include reduced injection frequency, reduced peak-valley profile, increased patient compliance, and reduced potential for undesired immune response.

Non-limiting examples of therapeutic proteins that may benefit from extended half-life include recombinant blood factor concentrates and substitutes (e.g., for the treatment of blood factor deficiencies), recombinant granulocyte colony stimulating factor (e.g., for the treatment of neutropenia), recombinant glucagon-like peptide-1 (e.g., for the treatment of diabetes type II), and asparaginase (e.g., for the treatment of acute lymphoblastic leukemia). Thus, lipidated ELP-therapeutic protein fusions thereof are also provided.

### D. Compositions Including Polypeptides

### 1. Formulations

As discussed above, polypeptides including non-standard amino acids have a broad range of applications, including biomedical applications. Therefore, pharmaceutical compositions including a polypeptide having one or more instances of one or more non-standard amino acids are provided. Pharmaceutical compositions containing peptides or polypeptides may be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration. The compositions may also be administered using bioerodible inserts and may be delivered directly to an appropriate lymphoid tissue (e.g., spleen, lymph node, or mucosal-associated lymphoid tissue) or directly to an organ or tumor. The compositions can be formulated in dosage forms appropriate for each route of administration.

a. Formulations for Parenteral Administration

In a preferred embodiment, the disclosed compositions, including those containing peptides and polypeptides, are prepared in an aqueous solution, and can be delivered to subject in need therefore, for example, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions include sterile water, buffered saline (e.g., Tris-HCl, acetate, phosphate), pH and ionic strength; and optionally, additives such as detergents and solubilizing agents (e.g., TWEEN® 20, TWEEN 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

b. Controlled Delivery Polymeric Matrices

Compositions including a polypeptide having one or more instances of one or more non-standard amino acids can be administered in controlled release formulations. In some embodiments, the polypeptide is the controlled release agent and is used in combination with another active agent. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel. The matrix can also be incorporated into or onto a medical device to modulate an immune response, to prevent infection in an immunocompromised patient (such as an elderly person in which a catheter has been inserted or a premature child) or to aid in healing, as in the case of a matrix used to facilitate healing of pressure sores, decubitis ulcers, etc.

The matrices can be non-biodegradable or biodegradable matrices. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

Controlled release oral formulations may be desirable. Polypeptides can be incorporated into an inert matrix which permits release by either diffusion or leaching mechanisms, e.g., films or gums. Slowly disintegrating matrices may also be incorporated into the formulation. Another form of a controlled release is one in which the drug is enclosed in a semipermeable membrane which allows water to enter and push drug out through a single small opening due to osmotic effects. For oral formulations, the location of release may be the stomach, the small intestine (the duodenum, the jejunem, or the ileum), or the large intestine. Preferably, the release will avoid the deleterious effects of the stomach environment, either by protection of the active agent (or derivative) or by release of the active agent beyond the stomach environment, such as in the intestine. To ensure full gastric resistance an enteric coating (i.e, impermeable to at least pH 5.0) is essential.

The devices can be formulated for local release to treat the area of implantation or injection and typically deliver a dosage that is much less than the dosage for treatment of an entire body. The devices can also be formulated for systemic delivery. These can be implanted or injected subcutaneously.

c. Formulations for Enteral Administration

The polypeptides can also be formulated for oral delivery. Oral solid dosage forms are known to those skilled in the art. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 21st Ed. (2005, Lippincott, Williams & Wilins, Baltimore, Md. 21201) pages 889-964. The compositions may be prepared in liquid form, or may be in dried powder (e.g., lyophilized) form. Liposomal or polymeric encapsulation may be used to formulate the compositions. See also Marshall, K. In: Modern Pharmaceutics Edited by G. S. Banker and C. T. Rhodes Chapter 10, 1979. In general, the formulation will include the active agent and inert ingredients which protect the polypeptide in the stomach environment, and release of the biologically active material in the intestine.

Liquid dosage forms for oral administration, including pharmaceutically acceptable emulsions, solutions, suspensions, and syrups, may contain other components including inert diluents; adjuvants such as wetting agents, emulsifying and suspending agents; and sweetening, flavoring, and perfuming agents.

### 2. Devices

In some embodiments, a polypeptide including one or more instances of one or more non-standard amino acids is coated onto, or incorporated into, an object or device, for example a medical device. The device can be a device that is inserted into a subject transiently, or a device that is implanted permanently. In some embodiments, the device is a surgical device.

Examples of medical devices include, but are not limited to, needles, cannulas, catheters, shunts, balloons, and implants such as stents and valves.

In some embodiments, the polypeptide can be formulated to permit its incorporation onto the medical device. In some embodiments the polypeptide inhibitor or pharmaceutical composition thereof is formulated by including it within a coating on the medical device. There are various coatings that can be utilized such as, for example, polymer coatings that can release an active agent over a prescribed time period. The polypeptide can be the polymer, the active agent, or both. The polypeptide can be embedded directly within the medical device. In some embodiments, the polypeptide is coated onto or within the device in a delivery vehicle such as a microparticle or liposome that facilitates its release and delivery. In some embodiments, the polypeptide is miscible in the coating.

In some embodiments, the medical device is a vascular implant such as a stent. Stents are utilized in medicine to prevent or eliminate vascular restrictions. The implants may be inserted into a restricted vessel whereby the restricted vessel is widened. The experience with such vascular implants indicates that excessive growth of the adjacent cells results again in a restriction of the vessel particularly at the ends of the implants which results in reduced effectiveness of the implants. If a vascular implant is inserted into a human artery for the elimination of an arteriosclerotic stenosis, intimahyperplasia can occur within a year at the ends of the vascular implant and results in renewed stenosis.

In some embodiments, the stents are coated or loaded with a composition including a polypeptide including one or more instances or one or more non-standard polypeptides. Many stents are commercially available or otherwise know in the art.

The compositions, methods of making, methods of using, and other embodiments disclosed herein can be further understood through the following numbered paragraphs.

1. A method of restoring one or more reduced or degraded para-azido-phenylalanine (pAzF) residues in a polypeptide in need thereof comprising contacting the polypeptide with an effective amount of imidazole-1-sulfonyl azide (ISAz) to restore one or more of the reduced or degraded pAzF residues to pAzF therein.

2. The method of paragraph 1, wherein the contacting occurs under aqueous conditions.

3. The method of paragraphs 1 or 2, wherein the contacting occurs in the absence of organic solvents.

4. The method of any one of paragraphs 1-3, wherein the conditions are not effective to limit or prevent the conversion of amines at the N-terminus and/or lysine residues to azides.

5. The method of any of one of paragraphs 1-4 wherein the contacting occurs in pH of between about 6.0 and about 8.5 inclusive, or between about 6.5 and about 7.6 inclusive, or between about 7.0 and about 7.5 inclusive, or about 7.2, or 7.2

6. The method of any one of paragraphs 1-5, wherein the ISAz is in about 2 to about 500 inclusive, or between about 20 and 250 inclusive, or about 200, or 200 equivalents per molecule.

7. The method of any one of paragraphs 1-6, wherein the contacting is carried out for about 1 to about 150 hours, or about 2 to about 100 hours, or about 5 to about 90 hours, or about 10 to about 72 hours, or about 42, 72, or 90 hours.

8. The method of any one of paragraphs 1-7, wherein the polypeptide comprises between 1 and 500 residues that are either pAzF or reduced or degraded pAzF.

9. The method of paragraph 8, wherein at least 10, 15, 20, 25, 30, 35, 40, 45, 50, 55, 60, 65, 70, 75, 80, 85, 90, or 95 percent of the between 1 and 500 residues are reduced or degraded pAzF prior to the contacting.

10. The method of paragraphs 8 or 9, at least 95, 90, 85, 80, 75, 70, 65, 60, 55, or 50 percent of the between 1 and 500 residues are pAzF after the contacting.

11. The method of any one of paragraphs 1-9, wherein at least 10, 20, 30, 40, 50, 60, 70, 75, 80, 85, 90, 95, 96, 97, 98, 99 or 100 percent of reduced or degraded pAzF are restored to pAzF.

12. The method of any one of paragraphs 1-11, wherein the contacting occurs in a composition comprising a plurality of the polypeptide.

13. The method of any one of paragraphs 1-12, wherein the contacting occurs in a composition comprising a heterogeneous mixture of different polypeptides comprising one or more reduced or degraded pAzF residues.

14. The method of any one of paragraphs 1-13, wherein the reduced or degraded pAzF is p-amino-phenylalanine (pAF).

15. The method of any one of paragraphs 1-14, wherein the reduced or degraded pAzF is a degradation product of phosphoramidate (pnY).

16. The method of paragraph 15, wherein the reduced or degraded pnY is p-amino-phenylalanine (pAF).

17. The method of any one of paragraphs 1-16, wherein the polypeptide comprises or is an elastin-like polypeptide (ELP).

18. The method of any one of paragraphs 1-17, wherein the polypeptide is a fusion protein.

19. The method of any one of paragraphs 1-18, wherein the polypeptide comprise the amino acid sequence of SEQ ID NOS:17 or 18.

20. The method of any one of paragraphs 1-18, further comprising modifying the pAzF residues to include one or more moieties conjugated thereto.

21. The method of paragraph 20, wherein the modifying comprises a copper-catalyzed azide-alkyne cycloaddition (“click”), strain promoted azide-alkyne cycloaddition, or Staudinger ligation photocrosslinking.

22. The method of paragraphs 20 and 21, wherein the moiety is a lipid.

23. The method of paragraph 22, wherein the lipid is a fatty acid, optionally wherein the fatty acid is palmitic acid.

24. The method of paragraph 22 or 23, further comprising determining the serum half-life of the polypeptide.

25. The method of paragraphs 24, wherein determining the half-life comprises determining: (i) the half-life of unbound polypeptide, (ii) the half-life of serum albumin, and (iii) the binding affinity between the protein and albumin.

26. The method of paragraph 25, wherein the polypeptide is a fusion protein comprising an ELP comprising one or more lipid-conjugated pAzF residues and a therapeutic protein, optionally wherein the therapeutic protein is a recombinant blood factor concentrate or substitute, recombinant granulocyte colony stimulating factor, asparaginase, or GLP-1.

27. A method of testing the activity of a putative phosphatase comprising
(i) contacting a putative phosphatse with a polypeptide comprising one or more comprising one or more phosphoramidate (pnY) residues;
(ii) optionally selecting the phosphatase when it dephosphorylates pnY to form p-amino-phenylalanine (pAF); and
(iii) converting the pAF to pAzF according to the method of any one of paragraphs 1-16.

28. The method of paragraph 27, wherein the polypeptide comprising one or more phosphoramidate (pnY) residues is made according to a method comprising carrying out a Staudinger-phosphite ligation reaction on a pre-cursor polypeptide comprising one or more pAzF residues.

29. The method of paragraph 28, wherein the Staudinger-phosphite ligation reaction comprises contacting the polypeptide with an effective amount of tris(4-(2,5,8,11,14-pentaoxahexadecan-16-yloxy)-5-methoxy-2-nitrobenzyl) phosphite (TNBP).

30. The method of paragraphs 28 and 29 comprising a deprotection reaction.

31. The method of paragraph 30, wherein the deprotection reaction comprises exposing the polypeptide to UV light.

32. The method of paragraph 31, wherein the UV light is from a laser, LED, or sunlight.

33. The method of any one of paragraphs 28-32, wherein the reaction is carried out in an alkaline buffered aqueous solution.

34. The method of any one of paragraphs 27-33, further comprising utilizing the polypeptide as the subject of a binding affinity assay with a test polypeptide.

35. The method of any one of paragraphs 27-34, comprising carrying out the method two or more in parallel with different putative phospatases.

36. The method of any one of paragraphs 27-35, comprising selecting the phosphatase when it dephosphorylates pnY to form p-amino-phenylalanine (pAF).

37. The method of any one of paragraphs 1-36, preceded by a method of making the polypeptide comprising translation of mRNA encoding the polypeptide in a translation system comprising an aminoacyl tRNA synthetase (AARS) and a cognate tRNA that can be charged with pAzF by the AARS and who's anticodon can recognize a codon encoding the pAzF in the mRNA.

38. The method of paragraph 37, wherein translation is in genomically recoded organism (GRO) E. coli cells expressing the translation system.

39. The method of paragraph 38, wherein translation is in vitro, optionally in a GRO lysate.

40. The method of any one of paragraphs 37-39, wherein the AARS is selected from SEQ ID NOS:1-15.

41. A polypeptide manufactured according to the method of any one of paragraphs 1-40.

42. The polypeptide of paragraph 41 comprising at least 75, 80, 85, 90, 95, 96, 97, 98, or 99 percent incorporation of pAzF at the desired locations.

43. A plurality of the polypeptides of paragraphs 41 or 42.

44. The plurality of polypeptides of paragraph 43 comprising at least 75, 80, 85, 90, 95, 96, 97, 98, or 99 percent incorporation of pAzF at the desired locations across the entire plurality.

45. A heterologous mixture comprising two or more different pluralities according to paragraphs 43 or 44.

46. A composition comprising the polypeptide, plurality of polypeptides, or mixture of any one of paragraphs 41-45.

47. The composition of paragraph 46, wherein the composition is a cell lysate or subfraction thereof.

48. The composition of paragraph 44, wherein the composition is a pharmaceutical composition comprising a pharmaceutical acceptable carrier and is suitable for administration to a subject in need thereof.

49. A composition, use, or method according to any of the disclosure herein including, but not limited to, the description, the experimental examples, and/or the figures and their descriptions.

### EXAMPLES

### Example 1: Investigating the Origin of pAzF Reduction in Proteins

### Materials and Methods

### General Materials.

ChemBioDraw was used for drawing, displaying and characterizing chemical structures, substructures and reactions, ChemBioDraw Ultra 14.0.0.117, 2014, PerkinElmer Informatics. Calculator Plugins were used for structure property prediction and calculations including pKa estimations, Marvin 17.2.27.0, 2017, ChemAxon (www.chemaxon.com). All solvents were purchased from Fisher Scientific with certified ACS grade. Formic acid was purchased from J. T. Baker (Avantor Performance Materials, Center Valley, PA, USA). Ammonium acetate was purchased from Sigma Aldrich. Peptides were synthesized by the Tufts University Core Facility (Boston, MA, USA) or ChinaPeptides Co., Ltd. (Shanghai, China). Imidazole-1-sulfonyl azide (ISAz) was acquired from Sigma-Aldrich. It is imperative to pay careful attention during its synthesis, handling and storage, due its sensitivity and risk of explosion (Fischer, et al., J Org Chem, 77, 1760-4 (2012), Goddard-Borger and Stick, Org Lett, 9, 3797-800 (2007)).

### Strain and Plasmid Information.

Azide-containing proteins were produced in a fully recoded strain of E. coli (C321. ΔA) where all genomic TAG codons were recoded to TAA (Lajoie et al., Science, 342, 357-60 (2013)). This strain contained a plasmid containing an inducible araBAD promoter and a constitutive copy of the pAcFRS.1.t1 synthetase (Amiram et al., Nat Biotechnol 33, 1272-1279 (2015)) and a constitutive tRNA_{CUA}. Together, the pAcFRS.1.t1 synthetase and tRNA_{CUA} comprise the orthogonal translation system (OTS) used in this study (Table 3). All GFP constructs are on a plasmid containing the pBR322 origin and are under the control of the PLtetO promoter induced with anhydrotetracycline.

TABLE 3
Description of plasmids for the orthogonal translational
system and the ELP-GFP reporter proteins. Plasmid characteristics
and gene context is given for each plasmid.

| | Origin of | | |
|---|---|---|---|
| Plasmid | Replication | Gene | Promoter |
| pEvol-OTS | P15A | pAcFRS.1.t1 | Ara |
| | | pAcFRS.1.t1 | Constitutive |
| | | tRNA | proK |
| | | chloramphenicol | Constitutive |
| | | acetyltransferase | |
| ELP-GFP | pBR322 | ELP-GFP | PLtetO |
| | | kanamycin resistance | Constitutive |

Expression and Purification of sfGFP Constructs.

Cultures were grown in the presence kanamycin and chloramphenicol (of 30 μg/ml each) at 34° C. in Luria-Bertani medium (American Bioanalytical, Natick, MA, USA). Cultures for the expression of azido-containing proteins were supplemented with 1 mM p-azido-L-phenylalanine (Bachem, Bubendorf, Switzerland) and 0.2% arabinose (Fisher Scientific, Hampton, NH, USA) for induction of the orthogonal translation system. GFP expression was induced at OD 0.4-0.6 using 60 ng/ml anhydrotetracycline and allowed to grow overnight. Pellets from ELP-protein expression were lysed by sonication and cleared with 1-2% polyethylenimine. Next, the samples were purified by salting out with 0.1-0.2 g/mL of sodium citrate, and applying heat/cooling cycles between 75° C. and ice. As comparison, cells were lysed with Bugbuster (EMD Millipore), and purified C-terminal His₆-tagged constructs with Ni-NTA His Bind Resin (Sigma-Aldrich). For both purification methods the resulting protein was confirmed to be >95% pure by Coomassie Brilliant Blue stained Bio-Rad Mini-PROTEAN TGX gel. For the time-course measurements, similar induction protocols were used. A 1 L culture was grown and at specified time-points 50 mL of culture were collected. Proteins were isolated using Bugbuster (EMD Millipore) followed by His-purification.

### Analytical Methods for Amino Acid, Peptide and Protein Analysis.

Protein samples were digested either with trypsin or thermolysin. For trypsinization, buffer exchange was carried out in 100 mM NH₄HCO₃ pH 7.8 or diluted 1:10 into this buffer, and an overnight treatment was applied at 37° C. with trypsin (Promega, Sequencing Grade Modified Trypsin (V511C)) to protein ratio of 1:80 or 1:40. For thermolysin treatments, the samples were first buffer-exchanged to 50 mM Tris buffer at pH 8.0, containing 0.5 mM CaCl₂). Subsequently, Thermolysin (Promega, V4001) was added at a ratio of 1:10-1:20 and samples were incubated for 5 h at 80° C.

High-resolution mass spectrometry (HRMS) data was collected using an Agilent iFunnel 6550 Quadrupole Time-Of-Flight (QTOF) MS with an electrospray ionization (ESI) source, coupled to an Agilent Infinity 1290 ultra-high-performance liquid chromatography (UHPLC) system with an Agilent Eclipse Plus C₁₈ 1.8 μm, 4.6×50 mm column. Solvents used were (solvent A) water 0.1% formic acid and (solvent B) CH₃CN 0.1% formic acid. Mass spectra were gathered using Dual Agilent Jet Stream (AJS) ESI in positive mode. The mass range was set from 110 to 1700 m/z with a scan speed of 3 scan/second. The capillary and nozzle voltages were set to 5500 and 2000 V, respectively. The source parameters were set with a gas temperature of 280° C. and a flowrate of 11 L/min, nebulizer at 40 psig, and sheath gas temperature at 350° C. at a flow of 11 L/min. MS data were acquired with MassHunter Workstation Data Acquistion (Version B.06.01, Agilent Technologies) and analyzed using MassHunter Qualitative Analysis (Version B.07.00, Agilent Technologies). Protein quantification was performed using peptide standards with the same sequence as the digested peptides, or based on calibration curves produced using dilutions series of peptide(1TAG)-GFP.

For MS/MS analysis, an Agilent 1290 UHPLC system coupled with a 6490 triple quadrupole MS was used. The LC system was coupled to an Agilent Eclipse Plus Cis 1.8 μm, 4.6×50 mm column. The MS was equipped with an Agilent AJS ESI source, operated in the positive ion mode. Nitrogen gas was used for nebulization, desolvation, and collision. A product ion scan was performed using a mass range from 100 to 1400 m/z and step size of 0.1 amu. The capillary voltage was set to 3000 V. The source parameters were set with a gas temperature of 220° C. and a flowrate of 19 L/min, nebulizer at 20 psig, and sheath gas temperature at 250° C. at a flow of 11 L/min. The fragmentor voltage and collision energy were set to 380 and 25 V, respectively. MS data were acquired with MassHunter Workstation Data Acquistion (Version B.06.01) and analyzed using MassHunter Qualitative Analysis (Version B.07.00).

In Vitro tRNA Charging.

The pAcFRS.1.t1 synthetase was His-purified, and stored in 50 mM KCl, 50 mM HEPES-KOH at pH 7.5, 10% glycerol. The cognate tRNA was in vitro transcribed (IVT) from dsDNA template containing a T7 promoter, hammerhead ribozyme and tRNA, as described previously(Fechter et al., 1998). The IVT buffer consists of 40 mM Tris-HCl at pH8.0, 0.01% Triton X, 30 mM MgCl₂, 2 mM Spermidine, 1 mM DTT. For a 100 μL reaction, 4 mM riboNTPs (N0466S, NEB), 100U T7 (M0251S, NEB) and 1-2 μM dsDNA template were added. The reaction was incubated at 37° C. for 3 h. Afterwards, the sample was diluted 5× with reaction buffer, and heated to 65C for 1 h to promote hammerhead cleavage. After a phenol:chloroform extraction, the cleaved tRNA was separated from other fragments using a TBE-Urea gel. Finally, the tRNA was refolded by slowly cooling the sample from 95° C. to r.t. At 65° C., MgCl₂ was added to a final concentration of 10 mM.

aaRS specificity was measured using in vitro charging. The reaction composition was 150 mM HEPES-KOH at pH 7.5, 10 mM MgCl₂, 2.5 mM DTT, 0.2 mg/mL BSA, 2 mM ATP, 1 mM amino acid, 500 nM aaRS and 750 nM tRNA. The reactions were incubated at 37° C. for 30 min. Afterwards, they were quenched using loading buffer (0.1 M sodium acetate pH 5.0, 8 M urea, 0.05% bromophenol blue, 0.05% xylene cyanol FF). The samples were then analyzed on an acid-urea PAGE gel (8 M urea, 0.1 M sodium acetate at pH 5.2) (Varshney et al., 1991), that was prerun in running buffer (0.1 M NaOAc at pH 5.2) for 20 min at 13 W. After sample loading, the gel was run at a constant 13 W for 20 h. The tRNA was visualized with ethidium bromide staining.

tRNA purification and amino acid quantification.

To evaluate in vivo tRNA charging with nsAA, bulk charged tRNA was collected (Varshney et al., J Biol Chem, 266, 24712-8 (1991)). Cells were harvested at an OD600 of 1.0. The cells were pelleted and resuspended in lysis buffer (0.3 M sodium acetate, 10 mM EDTA, pH 4.5) and mixed with an equal volume phenol:chloroform (pH 4.5). Cells were lysed by vortexing 3×30 seconds. The aqueous fraction was washed two more times with phenol:chloroform. The bulk tRNA was washed 2 times by ethanol precipitation. The RNA pellet was resuspended in 20 mM ammonium formate at pH 10, and incubated for 1 h at 37° C. to deacylate the tRNAs. Finally, the samples were analyzed by HRMS and quantified using calibration curves of the same amino acids. Further details are found in section Analytical methods for amino acid, peptide and protein analysis.

DNA Protein Sequences.
Peptide(1TAG)-GFP:
(SEQ ID NO: 23)
ATGAAAGGTCGTGACTCTGAAGGTCACCTGTAGACCGTTCCGATCCGTGAA
GGTAAAGGTGGCGGTAGCGGCAGCAAGGGCGAAGAACTGTTTACGGGCGTG
GTGCCGATTCTGGTGGAACTGGATGGTGATGTCAATGGTCACAAATTCAGC
GTGCGCGGCGAAGGTGAAGGCGATGCAACCAATGGTAAACTGACGCTGAAG
TTTATTTGCACCACGGGTAAACTGCCGGTTCCGTGGCCGACCCTGGTCACC
ACGCTGACGTATGGTGTTCAGTGTTTCAGTCGTTACCCGGATCACATGAAA
CGCCACGACTTTTTCAAGTCCGCGATGCCGGAAGGTTATGTCCAAGAACGT
ACCATCTCATTTAAAGATGACGGCACCTACAAAACGCGCGCCGAAGTGAAA
TTCGAAGGTGATACGCTGGTTAACCGTATTGAACTGAAAGGCATCGATTTT
AAGGAAGACGGTAATATTCTGGGCCATAAACTGGAATATAACTTCAATTCG
CACAACGTGTACATCACCGCAGATAAGCAGAAGAACGGTATCAAGGCTAAC
TTCAAGATCCGCCATAATGTGGAAGATGGCAGCGTTCAACTGGCCGACCAC
TATCAGCAAAACACCCCGATTGGTGATGGCCCGGTCCTGCTGCCGGACAAT
CATTACCTGAGCACGCAGTCTGTGCTGAGTAAAGATCCGAACGAAAAGCGT
GACCACATGGTCCTGCTGGAATTCGTGACCGCGGCCGGCATCACGCACGGT
ATGGACGAACTGTATAAAGGCTCACATCATCATCATCATCATTAATAA
ELP(OTAG)GFP:
(SEQ ID NO: 24)
ATGAGCAAAGGTCCCGGGGTTCCGGGTGGCGGCGTGCCGGGCGCAGGTGTT
CCGGGTTATGGTGTGCCGGGCGGCGGTGTCCCGGGTGCTGGTGTGCCGGGC
TACGGTGTCCCGGGTGGCGGTGTTCCGGGCGCTGGTGTCCCGGGTTATGGT
GTCCCGGGTGGCGGTGTTCCGGGTGCAGGCGTTCCGGGTTACGGCGTGCCG
GGCGGCGGTGTTCCGGGTGCTGGTGTGCCGGGCTATGGTGTCCCGGGTGGC
GGTGTGCCGGGCGCAGGTGTCCCGGGTTACGGTGTTCCGGGCGGCGGTGTC
CCGGGTGCAGGTGTGCCGGGCTATGGTGTTCCGGGTGGCGGGGTGCCGGGC
GCTGGTGTTCCGGGTTATGGTGTGCCGGGCGGCGGTGTCCCGGGTGCAGGT
GTGCCGGGCTACGGTGTCCCGGGTGGCGGTGTTCCGGGCGCAGGTGTCCCG
GGTTATGGGCCCGGCGGTGGGGGCAGCAAGGGCGAAGAACTGTTTACGGGC
GTGGTGCCGATTCTGGTGGAACTGGATGGTGATGTCAATGGTCACAAATTC
AGCGTGCGCGGCGAAGGTGAAGGCGATGCAACCAATGGTAAACTGACGCTG
AAGTTTATTTGCACCACGGGTAAACTGCCGGTTCCGTGGCCGACCCTGGTC
ACCACGCTGACGTATGGTGTTCAGTGTTTCAGTCGTTACCCGGATCACATG
AAACGCCACGACTTTTTCAAGTCCGCGATGCCGGAAGGTTATGTCCAAGAA
CGTACCATCTCATTTAAAGATGACGGCACCTACAAAACGCGCGCCGAAGTG
AAATTCGAAGGTGATACGCTGGTTAACCGTATTGAACTGAAAGGCATCGAT
TTTAAGGAAGACGGTAATATTCTGGGCCATAAACTGGAATATAACTTCAAT
TCGCACAACGTGTACATCACCGCAGATAAGCAGAAGAACGGTATCAAGGCT
AACTTCAAGATCCGCCATAATGTGGAAGATGGCAGCGTTCAACTGGCCGAC
CACTATCAGCAAAACACCCCGATTGGTGATGGCCCGGTCCTGCTGCCGGAC
AATCATTACCTGAGCACGCAGTCTGTGCTGAGTAAAGATCCGAACGAAAAG
CGTGACCACATGGTCCTGCTGGAATTCGTGACCGCGGCCGGCATCACGCAC
GGTATGGACGAACTGTATAAAGGCTCATAA
ELP(5TAG)GFP:
(SEQ ID NO: 25)
ATGAGCAAAGGTCCCGGGGTTCCGGGTGGCGGCGTTCCGGGCGCAGGTGTT
CCGGGTTAGGGTGTCCCGGGCGGCGGTGTCCCGGGTGCTGGTGTTCCGGGC
TACGGTGTCCCGGGTGGCGGTGTTCCGGGCGCTGGTGTCCCGGGTTAGGGT
GTCCCGGGTGGCGGTGTTCCGGGTGCAGGCGTTCCGGGTTACGGCGTCCCG
GGCGGCGGTGTTCCGGGTGCTGGTGTTCCGGGCTAGGGTGTCCCGGGTGGC
GGTGTCCCGGGCGCAGGTGTCCCGGGTTACGGTGTTCCGGGCGGCGGTGTC
CCGGGTGCAGGTGTTCCGGGCTAGGGTGTTCCGGGTGGCGGGGTCCCGGGC
GCTGGTGTTCCGGGTTATGGTGTTCCGGGCGGCGGTGTCCCGGGTGCAGGT
GTCCCGGGCTAGGGTGTCCCGGGTGGCGGTGTTCCGGGCGCAGGTGTCCCG
GGTTATGGGCCCGGCGGTGGGGGCAGCAAGGGCGAAGAACTGTTTACGGGC
GTGGTGCCGATTCTGGTGGAACTGGATGGTGATGTCAATGGTCACAAATTC
AGCGTGCGCGGCGAAGGTGAAGGCGATGCAACCAATGGTAAACTGACGCTG
AAGTTTATTTGCACCACGGGTAAACTGCCGGTTCCGTGGCCGACCCTGGTC
ACCACGCTGACGTATGGTGTTCAGTGTTTCAGTCGTTACCCGGATCACATG
AAACGCCACGACTTTTTCAAGTCCGCGATGCCGGAAGGTTATGTCCAAGAA
CGTACCATCTCATTTAAAGATGACGGCACCTACAAAACGCGCGCCGAAGTG
AAATTCGAAGGTGATACGCTGGTTAACCGTATTGAACTGAAAGGCATCGAT
TTTAAGGAAGACGGTAATATTCTGGGCCATAAACTGGAATATAACTTCAAT
TCGCACAACGTGTACATCACCGCAGATAAGCAGAAGAACGGTATCAAGGCT
AACTTCAAGATCCGCCATAATGTGGAAGATGGCAGCGTTCAACTGGCCGAC
CACTATCAGCAAAACACCCCGATTGGTGATGGCCCGGTCCTGCTGCCGGAC
AATCATTACCTGAGCACGCAGTCTGTGCTGAGTAAAGATCCGAACGAAAAG
CGTGACCACATGGTCCTGCTGGAATTCGTGACCGCGGCCGGCATCACGCAC
GGTATGGACGAACTGTATAAAGGCTCATAA
ELP(10TAG)GFP:
(SEQ ID NO: 26)
ATGAGCAAAGGTCCCGGGGTTCCGGGTGGCGGCGTGCCGGGCGCAGGTGTT
CCGGGTTAGGGTGTGCCGGGCGGCGGTGTCCCGGGTGCTGGTGTGCCGGGC
TAGGGTGTCCCGGGTGGCGGTGTTCCGGGCGCTGGTGTCCCGGGTTAGGGT
GTCCCGGGTGGCGGTGTTCCGGGTGCAGGCGTTCCGGGTTAGGGCGTGCCG
GGCGGCGGTGTTCCGGGTGCTGGTGTGCCGGGCTAGGGTGTCCCGGGTGGC
GGTGTGCCGGGCGCAGGTGTCCCGGGTTAGGGTGTTCCGGGCGGCGGTGTC
CCGGGTGCAGGTGTGCCGGGCTAGGGTGTTCCGGGTGGCGGGGTGCCGGGC
GCTGGTGTTCCGGGTTAGGGTGTGCCGGGCGGCGGTGTCCCGGGTGCAGGT
GTGCCGGGCTAGGGTGTCCCGGGTGGCGGTGTTCCGGGCGCAGGTGTCCCG
GGTTAGGGGCCCGGCGGTGGGGGCAGCAAGGGCGAAGAACTGTTTACGGGC
GTGGTGCCGATTCTGGTGGAACTGGATGGTGATGTCAATGGTCACAAATTC
AGCGTGCGCGGCGAAGGTGAAGGCGATGCAACCAATGGTAAACTGACGCTG
AAGTTTATTTGCACCACGGGTAAACTGCCGGTTCCGTGGCCGACCCTGGTC
ACCACGCTGACGTATGGTGTTCAGTGTTTCAGTCGTTACCCGGATCACATG
AAACGCCACGACTTTTTCAAGTCCGCGATGCCGGAAGGTTATGTCCAAGAA
CGTACCATCTCATTTAAAGATGACGGCACCTACAAAACGCGCGCCGAAGTG
AAATTCGAAGGTGATACGCTGGTTAACCGTATTGAACTGAAAGGCATCGAT
TTTAAGGAAGACGGTAATATTCTGGGCCATAAACTGGAATATAACTTCAAT
TCGCACAACGTGTACATCACCGCAGATAAGCAGAAGAACGGTATCAAGGCT
AACTTCAAGATCCGCCATAATGTGGAAGATGGCAGCGTTCAACTGGCCGAC
CACTATCAGCAAAACACCCCGATTGGTGATGGCCCGGTCCTGCTGCCGGAC
AATCATTACCTGAGCACGCAGTCTGTGCTGAGTAAAGATCCGAACGAAAAG
CGTGACCACATGGTCCTGCTGGAATTCGTGACCGCGGCCGGCATCACGCAC
GGTATGGACGAACTGTATAAAGGCTCATAA

### Results

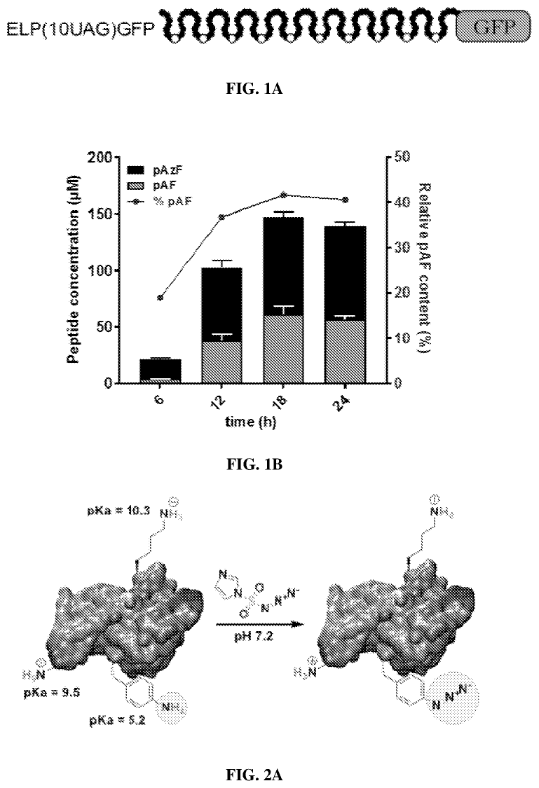

Experiments were designed to characterize the magnitude of pAF impurity in the genomically recoded strain of E. coli (Isaacs et al., Science, 333, 348-53 (2011), Lajoie et al., Science, 342, 357-60 (2013)). A previously reported gene encoding an elastin-like polypeptide (ELP) with 10 UAG codons fused to a GFP (ELP(10 UAG)GFP) was utilized, such that each repeating 15-mer in the ELP contains a single UAG codon capable of encoding pAzF (FIG. 1A) (Amiram et al., Nat Biotechnol, 33, 1272-1279 (2015)). After expression and purification, this reporter was digested with thermolysin to yield 10 identical peptides that can be analyzed by high-resolution mass spectrometry (HRMS) (peptides 1, 2 and 3; Table 4). The relative intensity of extracted-ion chromatograms for pAzF and pAF containing peptides (1 and 2, resp.) were similar to previously reported results (Wang et al., Nat Chem, 6, 393-403 (2014)). The resulting peptides were quantified using calibration curves derived from peptide standards (Table 4). To study the level of pAzF reduction in more depth, samples were collected at different times during protein expression. Results showed that relative pAF content increased over time, and 35-45% of pAF was observed in proteins after a typical expression protocol (FIG. 1B). The resulting combination of pAF and pAzF in the final protein impedes subsequent efforts to generate homogeneous polymers with multi-site functionalization across all pAzF sites.

TABLE 4
Characterization of the peptides used in this study to determine
pAzF reduction, analyze ISAz reactivity and as standards for
quantitation of tryptic peptides. n.a. = not applicable.

| Peptide | | | MW | | Purity |
|---|---|---|---|---|---|
| ID | Peptide name | Sequence | (g/mol) | Experimental m/z | (%) |
| 1 | ELP(pAF) | AGVPG(pAF)GVPGGG | 1238.6 | [M + 2H]2+ = 620.3 | 97 |
| | | VPG (SEQ ID NO: 27) | | | |
| 2 | ELP(pAzF) | AGVPG(pAzF)GVPGG | 1264.6 | [M + 2H]2+ = 633.3 | 97 |
| | | GVPG (SEQ ID NO: 28) | | | |
| 3 | ELP(Tyr) | AGVPGYGVPGGGVPG | 1239.6 | [M + 2H]2+ = 620.8 | 96 |
| | | (SEQ ID NO: 29) | | | |
| 4 | Peptide(pAF) | DSEGHL(pAF)TVPIRE | 1513.7 | [M + 2H]2+ = 757.9 | 98 |
| | | (SEQ ID NO: 30) | | | |
| 5 | Peptide(pAzF) | DSEGHL(pAzF)TVPIRE | 1539.7 | [M + 2H]2+ = 770.9 | n.a. |
| | | (SEQ ID NO: 31) | | | |

If pAF is present in the protein, it may originate from (1) mis-incorporation by a tRNA-aaRS system during translation, (2) post-translational reduction of pAzF, (3) reduction during purification, or (4) reduction due to MS analysis was investigated. To understand if reduction occurs prior to translation, experiments tested whether the OTS used in this work—pAcFRS.1.t1 (Amiram et al., Nat Biotechnol, 33, 1272-1279 (2015)) and amber suppressor tRNA_{AUC} derived from the archaea Methanocaldococcus jannaschii—is able to charge its cognate tRNA with pAF. Using purified pAcFRS.1.t1 in an in vitro charging assay (Varshney et al., J Biol Chem, 266, 24712-8 (1991)), pAzF was observed charged onto the tRNA_{AUC}, whereas no charging of pAF or tyrosine was observed. Similar results were found when evaluating the amino acid content derived from bulk charged tRNA isolated from the expression host after OTS induction (Varshney et al., J Biol Chem, 266, 24712-8 (1991)), with or without pAzF supplemented to the media. Quantification of charged amino acids revealed no signs of pAF, whereas pAzF was readily observed when supplemented. These findings were further corroborated by the observation that pAF is not incorporated into proteins when pAF is supplemented to the media in lieu of pAzF.

Experiments were designed to assess if reduction of pAzF residues occurs during protein purification or MS analysis. Experiments evaluated if pAzF is reduced over a range of pH (6.6, 7.2, 7.9) and temperatures (4, 20, 34, or 80° C.) used during sample purification, and found minimal (<1%) reduction to pAF under all conditions. Additionally, no reduction was seen during MS analysis of synthetic ELP(pAzF) peptide, demonstrating that the MS protocol does not cause reduction. These results, alongside the in vitro and in vivo tRNA charging experiments, indicate that reduction of pAzF to pAF occurs post-translationally in the cell. The cytoplasm of E. coli is known to be a reducing environment (Prinz et al., J Biol Chem, 272, 15661-7 (1997)), which impacts the redox state of certain protein residues (such as cysteines that form disulfide bonds or pAzF). Given the central importance of the cell's redox state, it would be challenging to fully prevent pAzF reduction through metabolic engineering (Lobstein, et al., Microb Cell Fact, 11, 56 (2012)). To maximize protein production and avoid potential growth impairment with mutagenized strains, an approach to revert the reduction of azides in vitro after protein purification was developed.

### Example 2: Restoring pAzF from pAF Using a Diazotransfer Reaction

### Materials and Methods

Diazotransfer reaction using imidazole-1-sulfonyl azide (ISAz).

Diazotransfer reactions were performed using different proportions of ISAz in 10×PBS (1.4 M NaCl, 0.1 M phosphate, 0.03 M KCl) or 100 mM NaPi at specified pH. Diazotransfer reactions with amino acids and peptides were stopped by lowering the pH with formic acid. In case of protein, the reaction was stopped by buffer exchange, and samples were subsequently digested. All samples were analyzed by HRMS as described in section Analytical methods for amino acid, peptide and protein analysis.

### Results

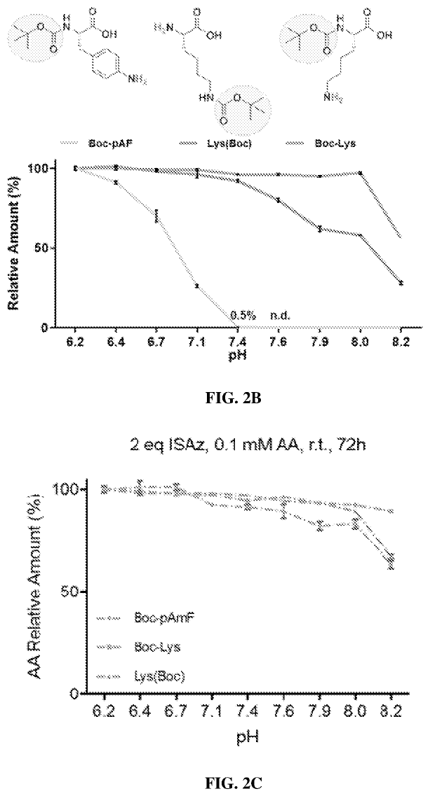

Experiments were designed to establish a diazotransfer reaction that can selectively restore the azide moiety at pAF residues in proteins while preserving the amine groups at the N-terminus and on lysines. Efforts focused on the protein-friendly diazotransfer reagent ISAz, whose activity towards primary amines has previously been shown to depend on the pH of the reaction (Schoffelen et al., Chemical Science, 2, 701-705 (2011), van Dongen et al., Bioconjugate Chemistry 20, 20-23 (2009)). Remarkably, there is almost a 2-unit difference between pAF's pKa 5.2 and the amines found in proteins, i.e., the pKas of lysine side-chains and the N-terminus are between 9.4-11.1 and 6.8-8.4, respectively (Thurlkill et al., Protein Sci, 15, 1214-8 (2006). It was considered that buffered reaction conditions above pAF's pKa may be capable of efficiently converting the amine in pAF to the azide yielding pAzF while minimizing side-reactivity at the N-terminus and lysines (FIG. 2A).

To establish conditions where selectivity and reactivity towards pAF are maximized, the reactivity of pAF was compared to the primary amines found at the N-terminus and lysines using N-tert-butoxycarbonyl-(Boc-) protected amino acids. Reactivity was analyzed by quantitative HRMS by measuring conversion of the primary amine to an azide. To optimize the reaction conditions, the number of equivalents of ISAz per molecule, pH, and duration of the reaction were investigated. When evaluating the reaction over a pH range (6.2-8.2) using 200 equiv. of ISAz at 20° C., it was found that the amino group in pAF reacts first, followed by the N-terminus, and then lysine side-chains at increasing pH (FIG. 2B). It was found that lowering the number of equivalents of ISAz reduces reaction efficiency and discrimination between the different amino groups (FIG. 2C). Under the conditions tested, optimal reactivity conditions were found, and based on these results, all subsequent experiments were preformed with 200 equiv. of ISAz at 20° C., for 72 hours, unless stated otherwise.

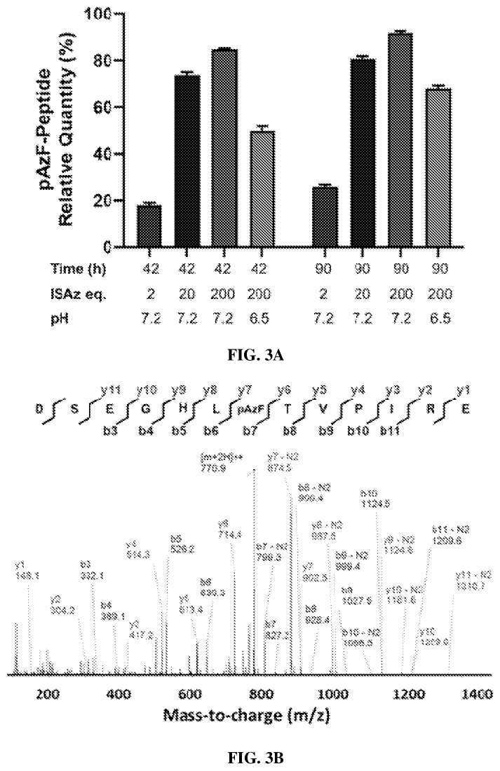

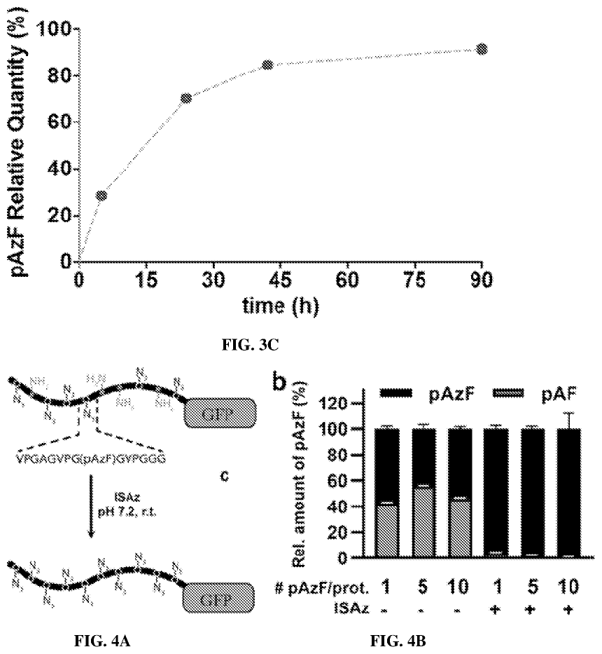

Next, experiments were designed to determine if the established conditions (200 equiv. ISAz, pH 7.2, 20° C., 72 h) could extend to a model peptide 4 (Table 4). pAzF regeneration was quantitatively examined over time by HPLC-HRMS, and compared the reactivity with conditions that could further minimize side-reactivity (i.e. lower ISAz equivalents, or lower pH). The diazotransfer reaction is significantly impaired when the ISAz equivalents are reduced to 2 (and to a lesser extend when using 20 equiv.), or when the pH is lowered to 6.5 (FIG. 3A). Conversely, when using the previously optimized conditions (200 equiv. of ISAz per molecule, pH 7.2, 20° C., 72 h) with amino acids, high conversions are observed: 84 and 92% of pAF is converted to pAzF at 42 h and 90 h, respectively (FIG. 3B; FIG. 3C). After the reaction, a single new peak is observed in the HPLC traces, which indicates a single product. Subsequent MS/MS analysis of the peak confirmed that fragments correspond only to the pAzF-containing peptide (peptide 5), whereas the N-terminal amine remains unaltered (FIG. 3B).

### Example 3: Selectively Restoring pAzF at Multiple Sites in Proteins

### Materials and Methods

Intact mass analysis of ISAz-treated proteins.

Intact MS experiments were performed by buffer exchanging the respective proteins to 200 mM ammonium acetate, pH 7.4, using a centrifugal buffer exchange device (Micro Bio-Spin column, Bio-Rad) (Hernandez and Robinson, 2007). The protein concentration of the buffer exchanged sample was kept between 5-10 μM. NativeMS was performed on a Q Exactive UHMR mass spectrometer (Thermo-Fisher Scientific) using in house nano ion-emitting capillaries. The ultra-high vacuum and the capillary voltage were set at 5.65e-10 mbar and 1.4 kV, respectively. Insource trapping voltage was adjusted between 50-150V to obtain the best quality spectra. The front-end S-lens rf was set to 100 to facilitate the transmission of the intact proteins. During spectral acquisition, 10 micro scans were summed up to a single scan to increase the signal to noise ratio. Relative quantitation of the proteoforms was obtained by combining the area under curves for each charge state and was simultaneously validated using UniDec (Reid et al., J Am Soc Mass Spectrom, 30, 118-127 (2019).

Proteomics analysis of ISAz side-reactivity

First, a set of proteins was reacted with ISAz as previously (200 equiv. of ISAz per molecule, 72 h) but at three pH conditions (7.2, 8.2 and 9.0). The proteins used include human ubiquitin (U-100H, Boston Biochem), bovine serum albumin (05470-1G, Sigma) and the ELP(10Tyr)-GFP used in this study. Next, proteins were digested with trypsin. The volume of 5 μg of protein (dissolved in 50 mM TEAB) was adjusted to 10 μl total using 50 mM TEAB (T7408-100ML, Sigma). 2.5 μl of 50 mM TEAB buffer with 5 mM EDTA, 25 mM TCEP and 0.62% Progenta ALS-110 were added. The samples were incubated for 20 min at 55° C. and immediately placed on ice to quench the reaction. Subsequently added was 1 μl of 200 mM MMTS (23011, Pierce) to cap cysteines and left at room temperature for 20 min. Next, 1.5 μl of 1 M TEAB were added and the sample was vortexed for 10 s. Subsequently, 0.5 μl of trypsin (0.5 μg/μl in 50 mM acetic acid) was added, mixed gently and spun down. Samples were incubated at 37° C. for 6 h and placed on ice to stop the reaction. The acid labile surfactant was cleaved by addition of 1.5 μl of 20% TFA. The reaction is left 15 min at room temperature and lyophilized. Samples were desalted using UltraMicroSpin C18 columns (SUM SS18V, The Nest Group).

Samples were analyzed using a NanoAquity UPLC (Waters) system using a 30 mm×150 μM ID trap column with a Kasil frit packed with ReproSil-Pur C18ΔQ 3 μM 200 Å resin and a 200 mm×75 μM ID self-packed Pico-frit column (New Objective) packed with 1.9 μM 120 Å ReproSil-Pur C18-AQ resin (both resins by Dr. Maisch) on a non-linear 90 min gradient from 5% CH₃CN 0.1% formic acid to 95% CH₃CN 0.1% formic acid with a flow rate of 300 nL/min and analyzed with an LTQ Orbitrap Velos (Thermo) using a Top 10 method. Mass spectrometry data was analyzed with Mascot(Perkins et al., 1999) and MaxQuant(Cox and Mann, 2008) using a custom database containing the sequences of the three proteins analyzed (human ubiquitin, bovine serum albumin and ELP(10Tyr)-GFP) in addition to the E. coli proteome (EcoCyc K-12 MG1655). The searches treated dithiomethane (Cys) as a fixed modification, oxidation (Met), deamidation (Asn, Gln), and ISAz-modified (Lys, N-terminus) as variable modifications. For Mascot, up to 5 missed trypsin cleavage events were allowed and the false discovery rate was set at 5%. For MaxQuant, up to 2 missed trypsin cleavage events were allowed, and peptides identified have a minimum length of 7 amino acids. The false discovery rate was set at 1%.

### Results

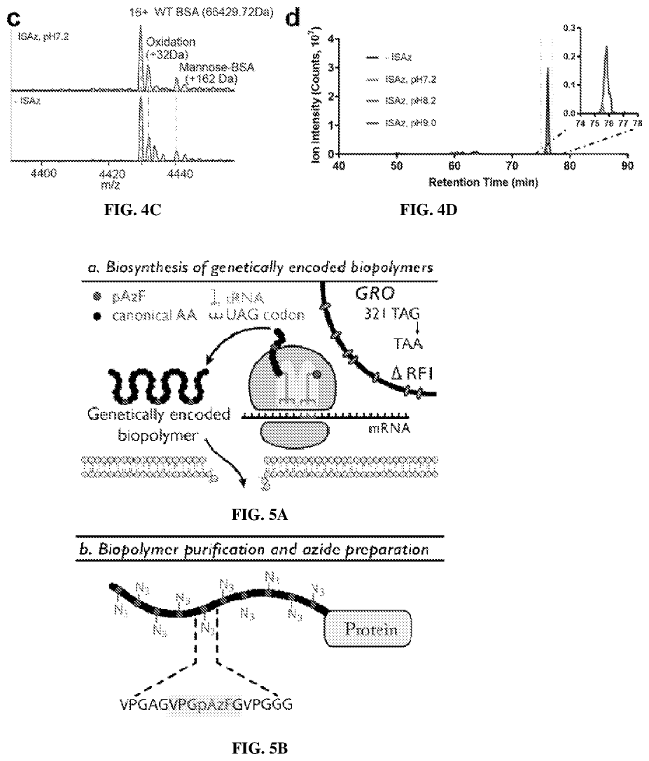

Next, the ability of ISAz to regenerate one, five or ten pAzF residues in proteins was examined (FIG. 4A). A peptide-GFP with one pAzF residue or ELP-GFP proteins with 5 or 10 pAzF residues (Amiram et al., Nat Biotechnol, 33, 1272-1279 (2015)). (ELP(5pAzF)-GFP and ELP(10pAzF)-GFP) were expressed and the residue composition of the digested ELP peptides was examined by HRMS. Based on calibration curves for both peptides (1 and 2), an initial content of 40-50% pAF was detected after protein purification (FIG. 4B). After performing the reaction with ISAz using the optimized conditions described above (200 equiv. of ISAz per molecule, pH 7.2, 20° C., 72 h), >95% conversion of pAF to pAZF was observed in the proteins encoded with 1, 5, or 10 nsAAs (FIG. 4B). These results demonstrate that ISAz can be used to revert pAF to pAzF residues in proteins at high efficiency.

The specificity of the ISAz treatment for pAF residues was characterized to assess any potential side-reactivity at the N-terminus or lysine residues. Previous work demonstrated minimal side-reactivity at ε-amines in lysine side-chains when using up to 175 equiv. of ISAz at pH 8.5 (Schoffelen et al., Chemical Science, 2, 701-705 (2011)). Thus, the side-reactivity at lysines may be lower, or undetectable, for the conditions optimized in this work (200 equiv. of ISAz per molecule, pH 7.2, 20° C., 72 h). To obtain a comprehensive understanding of where off-target reactivity could occur, modifications on three proteins (1) bovine serum albumin (BSA), (2) human ubiquitin (Ubq) and (3) ELP(10Tyr)-GFP were evaluated using two complementary approaches, intact protein mass spectrometry and LC-MS/MS of protein digests.

For intact protein mass spectrometry, high-resolution native mass spectra of untreated and ISAz-treated (pH 7.2) proteins were obtained. Two major species were observed for GFP prior to ISAz treatment, corresponding to GFP(pAF) and GFP(pAzF), at 30,066 Da and 30,092 Da, respectively. Treatment with ISAz largely converted pAF residues to pAzF, such GFP(pAzF) accounted for 89% of the total population, compared to 34% prior to treatment (FIG. 4C). These results are in good agreement with the data obtained from the quantitative MS analyses for digested peptides in FIG. 4B. In contrast, when we compared treated and untreated samples of BSA, Ubq, and ELP(10Tyr)GFP, which do not contain pAF residues, we found that their masses were in agreement with the theoretical masses of unmodified proteins (66430, 8564.43 and 39747.1 Da, respectively). An off-target diazotransfer reaction would correspond to a +26 Da addition, but this was not observed for BSA, Ubq, or ELP(10Tyr)GFP. Only small levels of commonly observed protein modifications, such as cysteine oxidation, Na⁺ binding, or acetylation of Ubq were detected (Charbaut, et al., FEBS Lett, 529, 341-5 (2002)). The presence of these minor species remained constant between untreated and treated samples, confirming they were not related to ISAz treatment. Finally, a lysate from cells expressing GFP(pAzF) was treated with ISAz to study the conjugation of a Cy5.5 alkyne fluorophore with click chemistry. A clear increase in Cy5.5 signal at GFP(pAzF) was observed, and minimal signal was detected at untargeted proteins.

To test if off-target modifications formed at lower abundance, LC-MS/MS proteomics analyses were performed on samples of BSA, Ubq and ELP(10Tyr)-GFP. Each protein was denatured and digested with trypsin and subsequently evaluated by mass spectrometry. The integrity of N-termini and lysine residues after treatment with 200 equiv. of ISAz at pH 7.2, 8.2 and 9.0 for 72 hours was compared in each of the three proteins. MaxQuant (Cox, Nat Biotechnol, 26, 1367-72 (2008)) and Mascot (Perkins et al., Electrophoresis, 20, 3551-67 (1999)) was used to identify modified residues and only found 4, 3 and 1 modified peptides with MaxQuant (and 10, 4 and 5 with Mascot) at pH 9.0 for BSA, Ub and ELP(10Tyr)-GFP, respectively. The observed peptides were further analyzed with Skyline (Schilling et al., Mol Cell Proteomics, 11, 202-14 (2012)) to assess the off-target reactivity. Only two peptides showed distinct EIC traces for the azido modification (FIG. 4D). Both peptides showed an increase in intensity of the modified peptide with increasing pH (and no detection in the untreated sample). The sparsity of modified peptides that were identified and the low signal strength at pH 7.2 indicate that side-reactivity is minimal using the ISAz method presented in this work.

In Examples 1-3, it was demonstrated that current efforts to functionalize proteins expressed with pAzF are hampered by significant levels of post-translational reduction to pAF. This has proven particularly challenging in proteins with multiple pAzF residues, where the reduction dramatically lowers the purity and results in heterogeneous protein product. To address this issue, a method is provided where treatment with ISAz in aqueous conditions and mild pH (7.2) is able to restore pAzF residues in both peptides and proteins to a purity greater than 95%. The low pKa of pAF in comparison to those of primary amines found in proteins (N-terminus and lysines) allows for selective recovery of pAzF, with nearly undetectable side reactivity. Even though, in some cases, the pKa of lysines may be lowered by its microenvironment, extensive reactivity was not found with proteomics analyses of ELP-GFP, BSA and Ubiquitin.

This work establishes a strategy for the production of proteins with multiple instances of pAzF for functionalization at high yield and purity. The combination of the GRO, in which UAG codons are dedicated to the incorporation of nsAAs (Lajoie et al., Science, 342, 357-60 (2013), highly efficient OTSs for pAzF (Amiram et al., Nat Biotechnol, 33, 1272-1279 (2015)), and chemical recovery of reduced pAzF residues allow robust functionalization through click-chemistry at multiple sites. This work sets the stage for genetically encoded biopolymers where diverse chemical moieties can be attached at specified residues to produce new genetically encoded biomaterials with tunable chemical and biophysical properties.

### Example 4: Design of Sequence-Defined Synthetic Biopolymers

To facilitate the biosynthesis of sequence-defined synthetic biopolymers with template-directed conjugation sites, a recently described synthetic biology expression system that allows efficient incorporation of pAzF at UAG codons (FIG. 5A) (Amiram et al., Nat Biotechnol 33, 1272-1279 (2015)) was utilized. This system is based on two key advances. First, the expression host is the genomically recoded organism (GRO) (Lajoie et al., Science 342, 357-360 (2013)), an E. coli MG1655 derivative, in which all instances of UAG stop codons were recoded to synonymous UAA codons, followed by the deletion of release factor 1 (RF1). This GRO establishes an open codon by eliminating competition between an orthogonal tRNA_{cuA}/aminoacyl tRNA synthase (aaRS) pair and termination at UAG codons by RF1. Second, aaRSs evolved for aminoacylation with nsAAs typically have significantly reduced activities compared to native enzymes, resulting in low levels of nsAA-tRNA and low yields for proteins with multiple instances of an nsAA (Umehara et al., FEBS Lett 586, 729-733 (2012)). Here, a tRNA_{cuA}/aaRS derived from M. jannaschii was utilized that was evolved for enhanced activity, enabling efficient multi-site incorporation of nsAAs into proteins (Amiram et al., Nat Biotechnol 33, 1272-1279 (2015)). Together, this expression system facilitates the biosynthesis of polypeptide polymers with multiple pAzF residues at high purity and yields (˜70 mg/L; see FIG. 5E).

To study the effect of the number of fatty acid conjugates on the in vivo serum half-life, the nsAA was introduced in an ELP with 10 consecutive pentadecapeptide repeats for functionalization. This ELP can serve as an independent polymer, or be appended directly to a protein for functionalization. Within each repeat, a designated guest position encodes either a tyrosine or a pAzF residue (FIG. 5B), such that the genetic template controls the number and position of pAzF residues in the ELP-GFP. In turn, the bioorthogonal copper(I)-catalyzed azide-alkyne Huisgen cycloaddition (click-chemistry) reaction between pAzF residues and fatty acid alkynes ensures site-specific conjugation (FIG. 5C-5D). Palmitic acid alkyne was used for functionalization because it had previously been shown to strongly promote binding to albumin (Kurtzhals et al., Biochem J 312 (Pt 3), 725-731 (1995)).

### Example 5: Fatty Acid Conjugation Controls Binding to Albumin

### Materials and Methods

### Strains and Protein Expression.

All proteins were expressed in the GRO (E. coli C321.ΔA, CP006698.1, GI:54981157) (Lajoie et al., Science 342, 357-360 (2013)) containing a previously described OTS plasmid—pAcFRS.1.t1—with a p15A origin of replication and chloramphenicol acetyltransferase selection marker (Amiram et al., Nat Biotechnol 33, 1272-1279 (2015)). The ELP-GFP genes were expressed from a plasmid with colE1 origin of replication and a kanamycin resistance marker. Each ELP-GFP construct had 10 repetitive units of 15 amino acids—VPGAGVPGXGVPGGG (SEQ ID NO:32)—where residue X is either tyrosine or pAzF (see below).

All cultures were grown at 34° C. under shaking (220 rpm). Before expression, the expression strains were grown to confluence in 50 mL 2xYT media. This culture was used to inoculate 1 L of 2xYT, containing 30 μg/mL chloramphenicol, 20 μg/mL kanamycin, 0.2% arabinose, and 1 mM pAzF. After 4 h, expression of ELP-GFP was induced, using a final concentration of 60 ng/mL anhydrotetracycline. Cells were harvested 24 hours after inoculation by centrifugation at 4,000 g for 15 minutes at 4° C.

The cell pellet was resuspended in PBS, pH7.4, and lysed by sonication (12 cycles of 10 s sonication separated by 40 s intervals, 40% amplitude). Poly(ethyleneimine) was added to each lysed suspension to a final concentration of 1.25%, after which the soluble fraction was separated from the cell debris by 15 minutes of centrifugation at 4,000 g. ELP-GFP proteins were then purified by phase transition triggered by sodium citrate, followed by centrifugation at 15,000 g for 3 minutes to eliminate contaminant proteins that did not precipitate. Finally, native E. coli proteins were denatured at 75° C., and removed by centrifugation. After three purification cycles, the ELP-GFP proteins to >95% purity as judged by Coomassie staining of SDS-PAGE gels.

### Protein Preparation and Functionalization.

When stated, para-aminophenylalanine (pAF) residues from ELP-GFP proteins were regenerated using imidazole-1-sulfonyl azide (ISAz). In brief, diazotransfer reactions were performed using 200 eq. of ISAz in 10×PBS (1.4 M NaCl, 0.1 M phosphate, 0.03 M KCl) pH 7.2 at room temperature. After 72 hours, reactions were stopped by exchanging the buffer to PBS (1×, pH7.4).

The ELP-GFP proteins were reacted with palmitic acid alkyne using copper(I)-catalyzed azide-alkyne Huisgen cycloaddition (click-chemistry). For this reaction, proteins were diluted to a final azide concentration of 30 μM, 35% DMSO, 0.16 mM palmitic acid alkyne, 0.1 mM CuSO₄ and 0.5 mM THPTA (premixed for 30 min), 5 mM aminoguanidine hydrochloride, and 5 mM sodium ascorbate. The click-chemistry reaction was incubated for 1 hour at room temperature under constant, gentle mixing. After the reaction, the protein was buffer exchanged to PBS (pH7.4) using amicon filters (10 kDa MWCO).

Proteins for biodistribution studies were further labeled with Alexa Fluor™ 647 NHS Succinimidyl Ester. Proteins were diluted to 0.1 mg/mL, and mixed with 5 μg/mL fluorophore in PBS for mild labeling. Excess dye was removed using amicon filters (10 kDa MWCO).

Endotoxins were removed from all protein preparations used for animal experiments, using Pierce™ high capacity endotoxin removal columns following the manufacturer's protocol (ThermoFisher Scientific; catalog #88274). Prior to injection, endotoxin levels were confirmed to be under 0.1 endotoxin unit (EU) per injection using Gel-Clot LAL reagent with sensitivity of 0.06 EU/mL (Charles River; catalog #R12006).

### Mass Spectrometry Characterization.

The purity at the guest residue was determined by quantitative mass spectrometry. The ELP-GFP proteins were buffer exchanged and diluted to 15 μM in digestion buffer (50 mM TRIS, pH 8.0, and 0.5 mM CaCl₂), and were digested with 1.5 μM thermolysin for 6 h at 80° C. The resulting ELP-peptides were quantified using standard curves based on synthetic peptides. High-resolution mass spectrometry (HRMS) data were collected using an Agilent iFunnel 6550 Quadrupole Time-Of-Flight (QTOF) MS with an electrospray ionization (ESI) source, coupled to an Agilent Infinity 1290 ultra-high-performance liquid chromatography (UHPLC) system with an Agilent Eclipse Plus C18 1.8 μm, 4.6×50 mm column. Solvents used were (solvent A) water 0.1% formic acid and (solvent B) CH₃CN 0.1% formic acid. Mass spectra were gathered using Dual Agilent Jet Stream (AJS) ESI in positive mode. The mass range was set from 110 to 1700 m/z with a scan speed of 3 scan/second. The capillary and nozzle voltages were set to 5500 and 2000 V, respectively. The source parameters were set with a gas temperature of 280° C. and a flowrate of 11 L/min, nebulizer at 40 psig, and sheath gas temperature at 350° C. at a flow of 11 L/min. MS data were acquired with MassHunter Workstation Data Acquisition (Version B.06.01, Agilent Technologies) and analyzed using MassHunterQualitative Analysis (Version B.07.00, Agilent Technologies).

### Intact Mass by MALDI-TOF.

For MALDI-TOF analysis, 2 μl of the protein samples were mixed in a ratio of 1:1:1 with 2% TFA solution and then with the matrix solution (375 μl of 20 mg/ml solution of 2,5-DHAP (2,5-Dihydroxy actetophenone) in ethanol and 125 μl of 18 mg/ml of aqueous DAC (Diammonium hydrogen citrate solution)) by pipetting, until crystallization of the mixture. 0.5 μl of the protein sample was then loaded on MALDI steel target plate and analyzed after solvent evaporation.

MALDI-TOF MS spectra were acquired using an MALDI-TOF/TOF autoflex speed mass spectrometer (Bruker Daltonilk GmbH, Bremen, Germany), equipped with a smartbeam-II solid-state laser (modified Nd:YAG laser, λ=355 nm), at the Ilse Katz Institute for Nanoscale Science and Technology (Ben-Gurion University of Negev, Beer-Sheva, Israel). The instrument was operated in positive ion, linear mode within a mass range from m/z 10 kDa to 50 kDa. Laser fluence were optimized for each sample. The laser was fired at a frequency of 1 kHz and spectra were accumulated in multiples of 500 laser shots, with 1500 shots in total. Calibration was performed using protein calibration standard from Bruker. Spectrum analysis was performed by the Flexanalysis software.

### Surface Plasmon Resonance.

Binding assays were performed on a Biacore T200 instrument. Human serum albumin (Sigma cat #A3782) or albumin from mouse serum (Sigma cat. #A3139) were immobilized by amine coupling to research grade CM5 chip (GEHealthcare, cat #BR100530) from 20 μg/ml solutions in 10 mM acetate pH 5.0. High density surfaces were created ranging from ˜1,300-12,800 RUs to minimize non-specific binding of ELP-GFP derivatives. Binding was measured with 60 s association phase and 600 s dissociation phase with either no regeneration, or surfaces were regenerated with two 30 s pulses of 50 mM NaOH. ELP-GFP derivatives were injected in duplicates from two-fold dilution series with at least 6 different concentrations ranging from −0.28 to 60 μM (depending on the polymer and its expected Kd); PBS was used as running buffer. Data were doubly-referenced against the signal collected on the reference cell and responses generated on the active cells during buffer injections. Data were analyzed using Evaluation software and fit into steady-state affinity binding model. Each reported affinity is an average from 4-8 independent measurements

TABLE 5
Amino acid sequence of reporter proteins used in this study.

| Protein name | Amino acid sequence |
|---|---|
| GFP-WT | SKGEELFTGVVPILVELDGDVNGHKFSVRGEGEGDATNGKLTLKFICTTGKLPVPWPTL |
| (superfolder) | VTTLTYGVQCFSRYPDHMKRHDFFKSAMPEGYVQERTISFKDDGTYKTRAEVKFEGDTL |
| | VNRIELKGIDFKEDGNILGHKLEYNFNSHNVYITADKQKNGIKANFKIRHNVEDGSVQL |
| | ADHYQQNTPIGDGPVLLPDNHYLSTQSVLSKDPNEKRDHMVLLEFVTAAGITHGMDELY |
| | KGS (SEQ ID NO: 22) |
| ELP(OUAG) | MSKGPG(VPGGGVPGAGVPGYG)₁₀PGGGG-(GFP-WT) (SEQ ID NO: 33) |
| GFP | |
| ELP(1UAG) | MSKGPG(VPGGGVPGAGVPG(pAzF)G)₁(VPGGGVPGAGVPGYG)₉PGGGG-(GFP-WT) |
| GFP | (SEQ ID NO: 34) |
| ELP(5UAG) | MSKGPG(VPGGGVPGAGVPG(pAzF)GVPGGGVPGAGVPGYG)₅PGGGG-(GFP-WT) |
| GFP | (SEQ ID NO: 35) |
| ELP(10UAG) | MSKGPG(VPGGGVPGAGVPG(pAzF)G)₁₀PGGGG-(GFP-WT) (SEQ ID NO: 36) |
| GFP | |

DNA sequence of ELP(OAUG)GFP used in this study.

Variable codons are highlighted/bolded.
(SEQ ID NO: 37)
ATGAGCAAAGGTCCCGGGGTTCCGGGTGGCGGCGTGCCGGGCGCAGGTGTT
CCGGGTTATGGTGTGCCGGGCGGCGGTGTCCCGGGTGCTGGTGTGCCGGGC
TACGGTGTCCCGGGTGGCGGTGTTCCGGGCGCTGGTGTCCCGGGTTATGGT
GTCCCGGGTGGCGGTGTTCCGGGTGCAGGCGTTCCGGGTTACGGCGTGCCG
GGCGGCGGTGTTCCGGGTGCTGGTGTGCCGGGCTATGGTGTCCCGGGTGGC
GGTGTGCCGGGCGCAGGTGTCCCGGGTTACGGTGTTCCGGGCGGCGGTGTC
CCGGGTGCAGGTGTGCCGGGCTATGGTGTTCCGGGTGGCGGGGTGCCGGGC
GCTGGTGTTCCGGGTTATGGTGTGCCGGGCGGCGGTGTCCCGGGTGCAGGT
GTGCCGGGCTACGGTGTCCCGGGTGGCGGTGTTCCGGGCGCAGGTGTCCCG
GGTTATGGGCCCGGCGGTGGGGGCAGCAAGGGCGAAGAACTGTTTACGGGC
GTGGTGCCGATTCTGGTGGAACTGGATGGTGATGTCAATGGTCACAAATTC
AGCGTGCGCGGCGAAGGTGAAGGCGATGCAACCAATGGTAAACTGACGCTG
AAGTTTATTTGCACCACGGGTAAACTGCCGGTTCCGTGGCCGACCCTGGTC
ACCACGCTGACGTATGGTGTTCAGTGTTTCAGTCGTTACCCGGATCACATG
AAACGCCACGACTTTTTCAAGTCCGCGATGCCGGAAGGTTATGTCCAAGAA
CGTACCATCTCATTTAAAGATGACGGCACCTACAAAACGCGCGCCGAAGTG
AAATTCGAAGGTGATACGCTGGTTAACCGTATTGAACTGAAAGGCATCGAT
TTTAAGGAAGACGGTAATATTCTGGGCCATAAACTGGAATATAACTTCAAT
TCGCACAACGTGTACATCACCGCAGATAAGCAGAAGAACGGTATCAAGGCT
AACTTCAAGATCCGCCATAATGTGGAAGATGGCAGCGTTCAACTGGCCGAC
CACTATCAGCAAAACACCCCGATTGGTGATGGCCCGGTCCTGCTGCCGGAC
AATCATTACCTGAGCACGCAGTCTGTGCTGAGTAAAGATCCGAACGAAAAG
CGTGACCACATGGTCCTGCTGGAATTCGTGACCGCGGCCGGCATCACGCAC
GGTATGGACGAACTGTATAAAGGCTCATAA

### Binomial Distribution of Protein Functionalization.

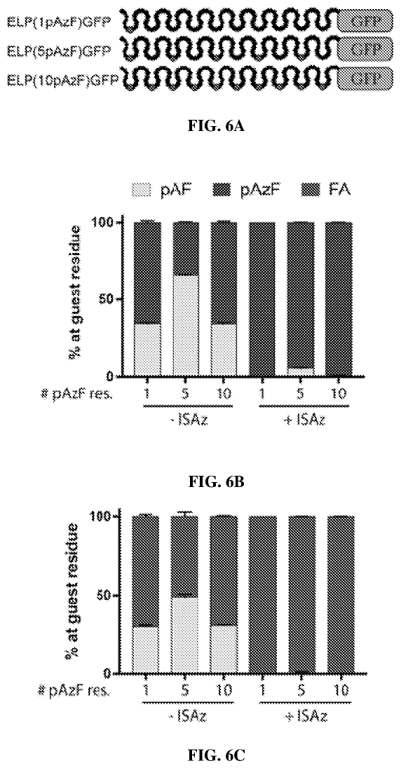

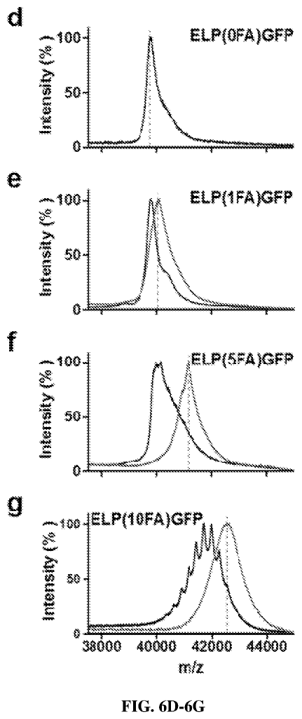

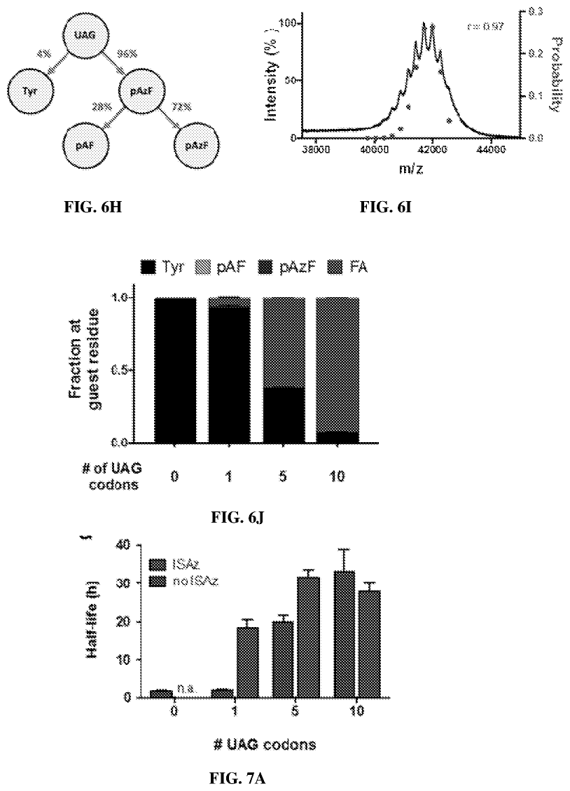

For the following predictions, it was assumed that each ELP(pAzF) unit is independent, and the probability of tyrosine misincorporation and pAzF reduction are unaffected by their position. As shown in FIGS. 6H and 6I, the probability of having an unreduced pAzF residue is determined by the probability of tyrosine misincorporation, p₁, and the probability of pAzF reduction, p₂.

The number of pAzF residues per protein will follow a binomial distribution, which can be described as

$$ℙ \{ X = k \} = ( \begin{matrix}n \\ k\end{matrix} ) {{p}^{k} ( 1 - p )}^{n - k} = {C}_{k}^{n} {{p}^{k} ( 1 - p )}^{n - k} = \frac{n !}{k ! ( n - k ) !} {{p}^{k} ( 1 - p )}^{n - k} .$$

Here, n represents the number of UAG codons per protein, and k is the number of those positions that contain unreduced pAzF. Finally, p is the probability that a UAG codon results in an unreduced pAzF residue. As such, p=p₁×p₂.

The probabilities p₁ and p₂ were derived from empirical data in this work (FIG. 6B, and see FIG. 6J). The resulting distribution was compared to intact mass spectrometry data of impure ELP(10 FA)GFP, and the correlation is reported.

### Results

To evaluate if proteins with a genetically controlled number of fatty acids could be produced, ELP-GFP were expressed with 0, 1, 5 or 10 UAG codons (FIG. 6A). To carefully examine the fidelity and efficiency of each step in the system, quantitative mass spectrometry (MS) analysis was performed of the ELPs digested with thermolysin, which liberates each of the 10 constituent ELP units. To account for differences in ionization efficiency between the different peptide species, ion counts were quantified using a standard curve for each peptide. First evaluated was the efficiency of pAzF incorporation and it was found that the abundance of ELP units with pAzF was directly proportional to the number of UAG codons in the construct (see FIG. 6J). Consistent with prior work (Amiram et al., Nat Biotechnol 33, 1272-1279 (2015)), when all 10 ELP units contained a UAG codon, minor (<5%) tyrosine misincorporation was detected.

While examining the fidelity of pAzF incorporation, significant levels of p-aminophenylalanine (pAF; FIG. 6B), the reduced form of pAzF, which cannot participate in click chemistry were observed. In this system, pAF is the result of pAzF reduction (Amiram et al., Nat Biotechnol 33, 1272-1279 (2015), Wang et al., Nat Chem 6, 393-403 (2014)) and causes significant impurities and heterogeneity in the final preparations if left unresolved. To overcome this impurity, the method discussed above (Examples 1-3) was utilized to selectively recover pAzF from pAF with imidazole-1-sulfonyl azide (ISAz). After treatment of ELP-GFP proteins with ISAz (see Methods: Protein preparation and functionalization), less than 5% of pAF in the polymer was observed (FIG. 6B).

Click chemistry was then used to attach alkynyl palmitic acid at the precise positions where pAzF was encoded and assessed the purity of each ELP-GFP construct. These functionalized constructs are denoted as ELP(nFA)GFP, where n indicates the number of UAG codons encoding pAzF in the template. All pAzF residues were converted to fatty acid-conjugates and no further reduction to pAF was detected during this reaction (FIG. 6C). To complement the quantification at the peptide level, Liquid Chromatography-Mass Spectrometry of the intact protein was used to evaluate the purity of the products (see methods: Intact Mass by MALDI-TOF). One dominant peak was consistently at the expected mass after ISAz treatment, whereas untreated samples demonstrated heterogeneous modification of the ELP-GFP (FIG. 6D-6G). For example, the ELP(10 FA)GFP without ISAz treatment showed multiple distinct peaks corresponding to an impure biopolymer with variable number of fatty acids. The peak profile correlates with a binomial distribution determined by the availability of pAzF residues (see FIGS. 6H-6I) and indicates pAzF reduction is probabilistic. Together, these results demonstrate that the genetically controlled placement of pAzF and chemical regeneration of reduced pAF residues facilitates the programmable and robust functionalization of biopolymers at high yield and purity.

Since prior work with single fatty acid conjugations of insulin showed that serum half-life is correlated with the binding affinity to albumin (Kurtzhals et al., Biochem J 312 (Pt 3), 725-731 (1995)), experiments were designed to determine if multi-site lipidation of ELP-GFPs would significantly enhance binding affinity to MSA, and consequently serum half-life, compared to a single conjugated fatty acid. To study the impact of increasing the number of fatty acids, ELP-GFP constructs (both with and without ISAz treatment) were analyzed with surface plasmon resonance (SPR). The K_{D} values of our constructs were estimated based on the steady state binding (Table 6). There was no detectable binding between MSA and the negative control without conjugated fatty acids (ELP(0FA)GFP). For untreated biopolymers, it was found that the K_{D} with a single fatty acid, ELP(1FA)GFP (K_{D}=126±32 μM), was enhanced 12- to 45-fold compared to ELP(5FA)GFP (K_{D}=10.4±4.0 μM) and ELP(10 FA)GFP (K_{D}=2.8±0.2 μM), respectively. For the ISAz-treated set, much stronger binding was observed overall: treated ELP(1FA)GFP presented a K_{D} of 25.9±7.1 μM, and an increase to 5 and 10 fatty acids per protein further lowered the K_{D} to 4.0±1.6 μM and 2.22±0.03 μM, respectively. These data indicate that the affinity for MSA is strongly enhanced by conjugation of multiple fatty acids per protein, and confirm that the binding affinity is correlated with the number of fatty acids.

TABLE 6
Binding affinity of ELP-GFP constructs for serum albumin.

| Construct | ISAz treated | K_{D} MSA (μM) | K_{D} HSA (μM) |
|---|---|---|---|
| ELP(0FA)GFP | No | n.d. | n.d. |
| ELP(1FA)GFP | No | 126 (±32.2) | n.a. |
| ELP(5FA)GFP | No | 10.3 (±4.0) | n.a. |
| ELP(10FA)GFP | No | 2.76 (±0.19) | n.a. |
| ELP(1FA)GFP | Yes | 25.9 (±7.1) | 19.3 (±3.9) |
| ELP(5FA)GFP | Yes | 4.0 (±1.6) | 3.16 (±0.60) |
| ELP(10FA)GFP | Yes | 2.22 (±0.03) | 1.64 (±0.17) |

The K_{D} values were calculated from the steady-state affinity binding model obtained by surface plasmon resonance (see methods: surface plasmon resonance). Mean and s.d. were derived from 4-8 independent experiments, n.d. indicates that no binding was detected, n.a. indicates not analyzed. MSA = mouse serum albumin; HSA = human serum albumin.

### Example 6: Functionalized Biopolymers Exhibit Tunable Half-Life in Mice

### Materials and Methods

### Mouse Models and In Vivo Experiments

All experiments were performed in C57BL/6 mice in accordance with the guidelines of the Animal Care and Use Committee of Yale University. Recommendations from the Guide for the Care and Use of Laboratory Animals (Institute of Laboratory Animal Resources, National Research Council, National Academy of Sciences, 1996) were followed during these experiments.

The half-lives of ELP-GFP constructs were calculated from concentrations measured from blood samples collected over the course of a week. The experiments were initiated by injecting 120 μL of 10 μM ELP-GFP intravenously or subcutaneously. At indicated times, 2 μL blood was collected from a tail puncture, and diluted 1:25 in heparin tubes. The blood sample was vortexed briefly and cells were pelleted by centrifugation (2 min at 14,000 g). The soluble fraction was collected and frozen at −20° C. until analysis. ELP-GFP concentrations of the samples were determined using a GFP ELISA Kit (Abcam, cat. #ab171581). The samples were diluted in PBS as needed, to ensure that the concentration fell within the quantifiable range of the standard curve.

To study the immunogenicity and biodistribution of ELP-GFP, 120 μL of 10 μM Alexa Fluor™ 647 labeled constructs were injected, and blood and organs were collected at indicated times. As positive control for an immune response, 100 μg LPS was injected, and an injection of PBS was performed as negative control. Organs were imaged using Amersham Imager 600 RGB. For cytokine quantification, blood was allowed to coagulate, and serum was collected. Cytokines were quantified from the serum samples using the BD CBA Mouse Inflammation Kit (Fisher Scientific, cat. #BD 552364).

### Results

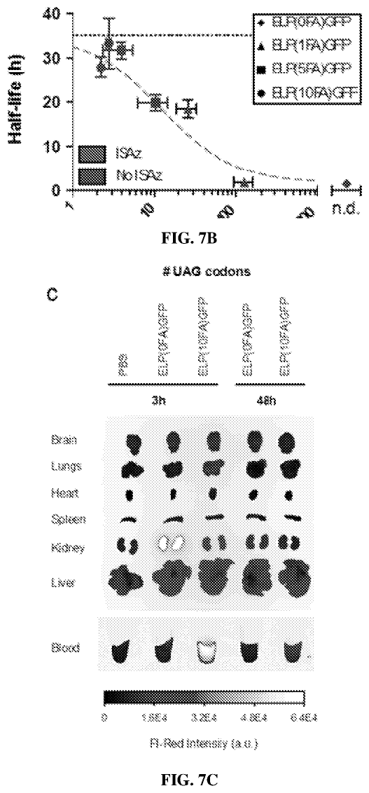

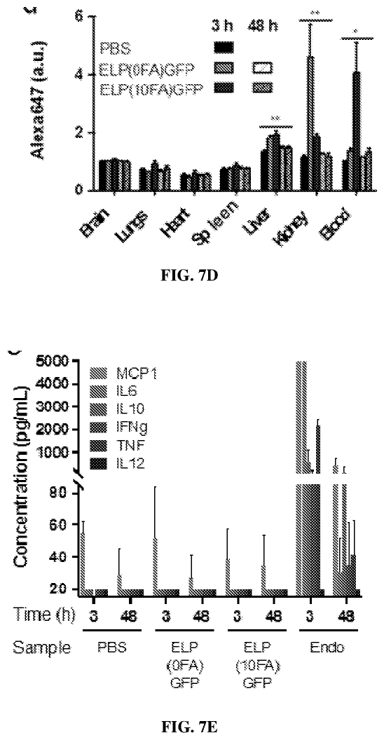

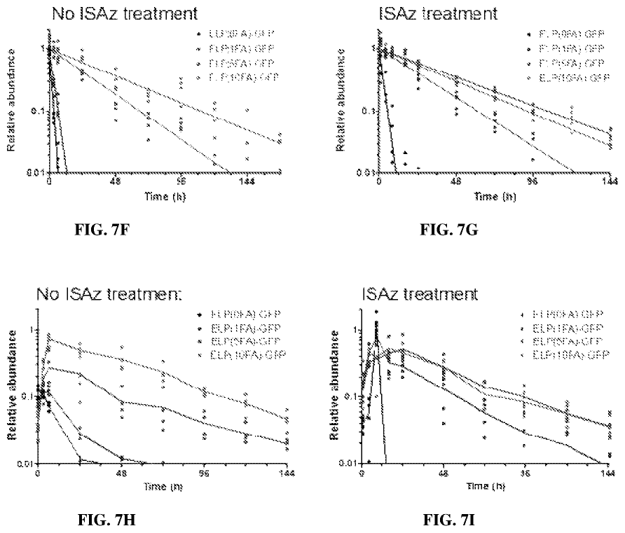

Next, experiments were designed to determine if the tighter binding affinity translates into prolonged half-life in C57BL/6J mice. A total of 50 μg of each protein variant (10 μM in PBS) was injected intravenously, and blood was collected after 1, 4, 8, 16 and 24 hours, followed by daily collections through seven days. The blood levels of ELP(nFA)-GFP constructs were measured using a GFP-specific ELISA, and their pharmacokinetic profiles were calculated (see FIGS. 7F and 7G). A 16-19-fold increase in half-life from 1.7 hours for ELP(0FA)GFP to 28-33 hours for ISAz-treated ELP(5FA)GFP and ELP(10FA)GFP, as well as for untreated ELP(10FA)GFP (FIG. 7A). Notably, when the same constructs were injected subcutaneously, a delayed peak concentration was observed, but the half-lives were equivalent to intravenous injections (see FIGS. 7H-7J). These data show that the half-life of tight binding ELP-GFP constructs with multiple fatty acid conjugates approaches the half-life of MSA in mice (35 hours), and is similar to the half-life of 28 hours reported for protein-MSA fusion proteins (Yang et al., Biomater Sci 6, 2092-2100 (2018)).

Based on the quantitative analysis of fatty acid conjugation of ISAz-treated and untreated constructs, ISAz-treated ELP(5FA)GFP and untreated ELP(10FA)GFP are likely to have a similar number of fatty acids per construct. This corresponds to their similar binding affinities to albumin and half-lives. In the case of ISAz-treated ELP(10FA)-GFP, a small decrease (although not statistically significant) in half-life was founnd compared to treated ELP(5FA)GFP and untreated ELP(10FA)GFP. One possibility is that denser packing of the 10 fatty acids does not improve or even reduce the availability of fatty acids for albumin binding, and this highlights the value of being able to precisely control the number of fatty acids per protein.

### Example 7: Model of Lipidized Biopolymers Kinetics and Serum Half-Life

### Materials and Methods

### Model of Half-Life Extension.

In this model, the predicted half-life is determined by the composite clearance of ELP-GFP and ELP-GFP bound to albumin. As such, the half-life is determined by two factors. First, it was supposed that free and bound ELP-GFP have differential clearance rates, where the half-life of free ELP-GFP is experimentally determined from ELP(0FA)GFP, and the half-life of bound ELP-GFP follows that of albumin. Second, the ratio of free and bound ELP-GFP is determined by the binding affinity between ELP-GFP and albumin. Collectively, these factors can be described by four ordinary differential equations:

$${r}_{1} = [ ELP ] \times [ Albumin ] \times {k}_{a}$$

$${r}_{2} = [ Complex ] \times {k}_{d}$$

$${r}_{3} = [ ELP ] \times \frac{ln ( 2 )}{{τ}_{ELP}}$$

$${r}_{4} = [ Complex ] \times \frac{ln ( 2 )}{{τ}_{Albumin}}$$

where kₐ is the association constant for binding between albumin and ELP-GFP, and k_{d} is the dissociation constant. The reaction r₁ represent the binding of ELP-GFP to albumin and is dependent on the concentrations of both. The binding is reversible, and r₂ expresses the dissociation of the complex. Furthermore, τ_{ELP} and τ_{Albumin} are the half-lives of unbound ELP(0FA)GFP and albumin, respectively. The reactions r₃ and r₄ describe the exponential decay of unbound ELP(0FA)-GFP and albumin, where τ_{ELP} and τ_{Albumin} are their respective half-lives.

The equations in this dynamical system allow calculation of the change of the three variables [ELP], [Albumin] and [Complex]:

$$\frac{d [ ELP ]}{dt} = - {r}_{1} + {r}_{2} - {r}_{3}$$

$$\frac{d [ Albumin ]}{dt} = - {r}_{1} + {r}_{2} + {r}_{4}$$

$$\frac{d [ Complex ]}{dt} = + {r}_{1} - {r}_{2} - {r}_{4}$$

In this system, the concentration of total albumin is kept constant by reintroducing unbound albumin equal to the amount of bound albumin that was degraded.

Starting concentrations for [Albumin], [ELP] and [Complex] were set to 250 uM, 10 uM and 0 uM, respectively. The system of differential equations was solved in python, using scipy.integrate.odeint, at is intervals for 144 h. The half-life was calculated using linear regression on the log transformed [ELP]ₜₒₜₐₗ (i.e., bound and unbound) after complex formation has reached equilibrium.

### Results

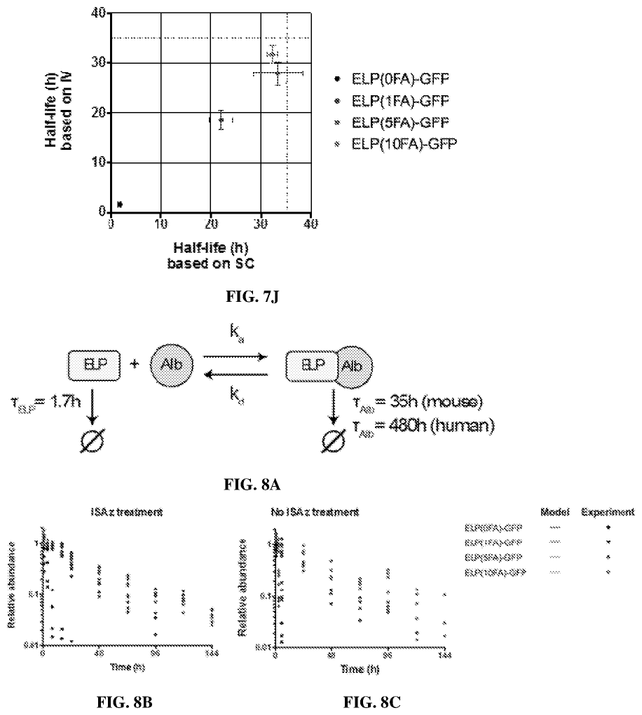

Computationally modeling of the system was used to gain a deeper understanding of the correlation between the binding affinity and half-life (see FIG. 8A-8C and Materials and Methods). In brief, a set of ordinary differential equations describes the binding and release of ELP-GFP from albumin as a function of the K_{D}, as well as the clearance of both bound and unbound ELP-GFP. Here, unbound ELP-GFP has a half-life of 1.7 hours, as empirically determined, and bound ELP-GFP is cleared at the same rate as albumin (35 hours) (Chaudhury et al., J Exp Med 197, 315-322 (2003)). Importantly, the half-life of the protein is determined by three parameters in this model: (i) the half-life of unbound protein, (ii) the half-life of serum albumin, and (iii) the binding affinity between the protein and albumin. By simulating the kinetics over time, the overall clearance rate were calculated, and predictions made by the model were in good agreement with the empirical measurements for K_{D} and half-life (FIG. 8B, and see FIGS. 8A-8C). This indicates predictive capability for the half-life based on empirically determined K_{D} values, or the model can provide a target K_{D} based on the desired half-life. Together, these results confirm that titrating the number of fatty acids allows predictable tuning of the protein half-life by modifying the binding affinity to albumin.

### Example 8: Functionalized Biopolymers are Biocompatible and Nonimmunogenic

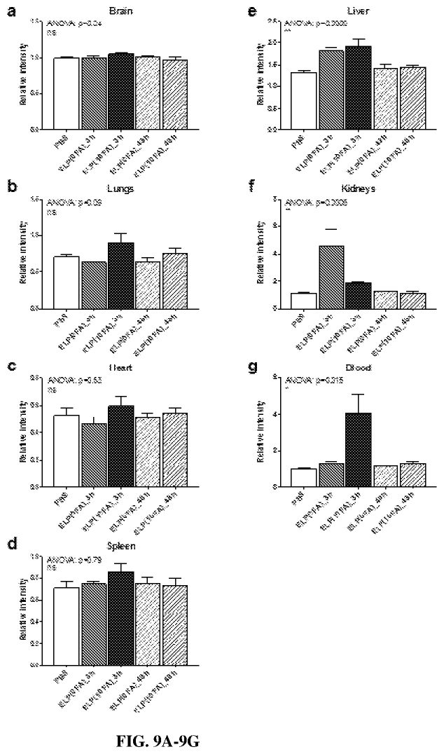

To evaluate the biocompatibility of the ELP-GFP constructs, biodistribution and inflammatory response were assessed. Alexa647-labeled ELP(0FA)GFP and ELP(10 FA)GFP were administered intravenously, and after 3 or 48 hours, the brain, lungs, heart, spleen, liver, kidneys and blood were collected and imaged for far-red fluorescence (FIG. 7C, and see FIGS. 9A-9G). In the case of ELP(0FA)-GFP, most of the reporter had cleared from the blood after three hours, and a strong signal was observed in the kidney, whereas ELP(10 FA)-GFP was clearly observed in the blood, and to a lesser extent in the kidney. After 48 hours, the Alexa647 signal was only observed in the blood of ELP(10 FA)-GFP-injected mice, whereas the intensity in all other organs had returned to the basal level seen in the PBS injection control. These results were consistent with a much faster clearance of ELP(0FA)-GFP, which is likely to occur mostly through excretion from the kidneys. The blood from each of these conditions was further analyzed for signs of inflammation. No elevation of pro-inflammatory cytokine levels after injection of ELP-GFP constructs was detected compared to PBS injection, whereas injection with LPS as positive control gave a clear immune response at both 3 and 48 hours (FIG. 7D). Together, these results show that fatty acid-conjugation allows half-life extension without long-term accumulation in organs, or eliciting an inflammatory response after intravenous injection.

Considering applications of this technology for peptide and protein drug delivery in humans, it was evaluated if the use of multiple fatty acids per protein conveyed similar increases in binding affinity to human serum albumin (HSA). K_{D} values of 19.3±3.9 μM, 3.2±0.6 μM and 1.6±0.2 μM were observed for pure ELP-GFP constructs with 1, 5 and 10 fatty acids per protein, respectively (Table 6). These binding affinities closely mirror the values observed for MSA, indicating that multi-site lipidation of proteins could be a promising strategy to tailor half-life in humans.

In Examples 4-8, the production of sequenced-defined synthetic biopolymers conjugated with a programmable number of fatty acids to tailor the serum half-life of proteins is provided. Specifically, the genetically encoded pAzF residues facilitate precise and programmable functionalization with fatty acids, which facilitates titration of the binding affinity to both MSA and HSA. The binding affinity to albumin was predictive of the serum half-life in mice, indicating that the protein clearance can be tuned by controlling the number of conjugated fatty acids per protein. Notably, serum half-lives of up to 33 hours was measured, which is 94% of the 35-hour half-life of MSA. Importantly, with similar binding affinities for MSA and HSA, it is believed that the half-life of these same constructs will be higher in humans, given that HSA has a significantly longer half-life (˜19 days) (Peters, Adv Protein Chem 37, 161-245 (1985)).

Lipidation is an appealing alternative to PEG, which has come under scrutiny due to concerns about immunogenicity of PEG (Ganson et al., Arthritis Res Ther 8, R12 (2006), Armstrong et al., Cancer 110, 103-111 (2007)) and uncertainty about its degradation and clearance from the body (Baumann et al., Drug Discov Today 19, 1623-1631 (2014)). The use of fatty acids has clinical precedence, offers greater tunability than direct fusion to albumin, and has a well-established safety profile (Menacho-Melgar et al., J Control Release 295, 1-12 (2019)). However, the usefulness of current lipidation strategies is constrained by two factors. First, typically only moderate half-life extensions are achieved due to weak binding of pharmaceuticals with single fatty acids to albumin. Second, the ability to identify uniquely reactive residues without impacting bioactivity remains challenging with conventional labeling strategies. This work addresses both limitations with a general methodology that supports tuning the half-life extension by titrating the number of fatty acids per protein, and the ability to design conjugation sites at monomeric precision permits facile screening of permissive residues to maintain bioactivity.

Unique to this work is the multisite and programmable placement of nsAAs to produce a biopolymer with tunable properties, permitted by sequence-defined insertion of multiple fatty acids per biopolymer for functionalization. Bioorthogonal conjugation sites (e.g., pAzF residues) allow the attachment of a wide variety of chemical moieties to expand the palate of biological chemistry far beyond fatty acids at genetically encoded positions throughout the protein to enhance its functionality. This establishes a foundation for a new class of synthetic, sequence-defined biopolymers composed of a combination of natural and synthetic monomers that unites the diversity of the chemical world with the monomeric precision of translation in biological systems. These biopolymers are facilitated by recoded organisms with open coding channels dedicated to the template-directed incorporation of synthetic monomers. This work, together with further recoding efforts to open up additional coding channels dedicated for multiple distinct nsAAs, establishes the basis for new and programmable biopolymers (Arranz-Gibert et al., Curr Opin Chem Biol 46, 203-211 (2018), Lutz et al., Science 341, 1238149 (2013)) with broad usefulness in biological research, pharmaceuticals, materials science, and biotechnology.

### Example 9: Site-Specific Incorporation of pnY Using Recoded E. Coli

### Materials and Methods

### General Materials.

ChemBioDraw was used for drawing, displaying and characterizing chemical structures, substructures and reactions, ChemBioDraw Ultra 14.0.0.117, 2014, PerkinElmer Informatics. Calculator Plugins were used for structure property prediction and calculations including pKa estimations, Marvin 17.2.27.0, 2017, ChemAxon (www.chemaxon.com). All solvents were purchased from Fisher Scientific with certified ACS grade. Formic acid was purchased from J. T. Baker (Avantor Performance Materials, Center Valley, PA, USA). Ammonium acetate was purchased from Sigma Aldrich. TNBP (tris(4-(2,5,8,11,14-pentaoxahexadecan-16-yloxy)-5-methoxy-2-nitrobenzyl) phosphite was synthesized by New England Discovery Partners (Branford, CT, USA) as previously reported.(Serwa et al.) Peptides were synthesized by the Tufts University Core Facility (Boston, MA, USA) or ChinaPeptides Co., Ltd. (Shanghai, China).

### Cloning.

All cloning was done using isothermal assembly (New England Biolabs, Ipswich, MA, USA) (Gibson et al., Nat Methods 6, 343-345 (2009)). Primers were acquired from the Keck Biotechnology Resource Laboratory (Yale University, New Haven, CT, USA). Polymerase chain reactions (PCR) for isothermal assembly were performed with KAPA HiFi PCR Kits (Kapa Biosystems, Wilmington, MA, USA), while sequencing was performed with KAPA HotStart ReadyMix (Kapa Biosystems), both according to manufacturer instructions. All sequencing was performed by Genewiz (South Plainfield, NJ, USA). Constructs were propagated in E. coli BL21(DE3). Transformations were performed by electroporation with a 1 mm gap electroporation cuvette (Bio-Rad, Hercules, MA, USA) with the following parameters: 1.8 kV, 200 V and 25 μF.

### Strain and Plasmid Information.

Azide-containing proteins were produced in a fully recoded strain of E. coli (C321.dprfA) where all genomic TAG codons were recoded to TAA. This strain contained a genomic copy of the pAcFRS.1.t1 synthetase (Amiram et al., Nat Biotechnol, 33, 1272-1279 (2015)) under the araBAD promoter and a constitutive tRNA_{CUA}. Together, the pAcFRS.1.t1 synthetase and tRNA_{CUA} comprise the orthogonal translation system (OTS) used in this study. All GFP constructs were cloned into a plasmid containing the pBR322 origin and were placed under the control of the PLtetO promoter induced with anhydrotetracycline (Lutz and Bujard, Nucleic Acids Res 25, 1203-1210 (1997)).

### Caveolin Epitope Design.

Recent work in E. coli phosphoproteomic analysis was utilized to minimize background tyrosine phosphorylation of a reporter epitope. E. coli phosphorylation occurs preferentially on sequences containing a +1 aspartate, −1 glycine, or −6/+3/+4/+5 lysine (Hansen et al., PLoS Pathog 9, e1003403 (2013)). The PhosphoSite database was used to identify human-derived peptides that have been frequently observed with tyrosine phosphorylation, but did not have elements of the E. coli phosphorylation motif (Hornbeck et al., Nucleic acids research 40, D261-270 (2012)). After sorting by PubMed references, the top three hits were EGFR Y1016, ZAP70 Y319, and caveolin-1 Tyr14. After manual curation, the first two were excluded due to proximity to E. coli motifs (DEV and SPY, respectively). The caveolin-1 Tyr14 motif was cloned to the N-terminus of superfolder GFP(Pedelacq et al., 2006) with either a TAG codon (Cav-pAzF) or a tyrosine suppressing codon (Cav-Tyr) at the tyrosine 14 position.

### DNA Sequences for Reporter Constructs.

The GFP reporters used in this study are fusion proteins of the Caveolin-1 Y14 epitope, a short linker, and superfolder GFP (sfGFP) (Pedelacq, et al., Nat Biotechnol 24, 79-88 (2006)). The TAG codon in the Cav-pAzF construct and the associated tyrosine codon in Cav-Tyr are bolded and underlined below.

Sequence for Cav-pAzF:
(SEQ ID NO: 38)
ATGAAAGGTCGTGACTCTGAAGGTCACCTGTAGACCGTTCCGATCCGTGAA
GGTAAAGGTGGCGGTAGCGGCAGCAAGGGCGAAGAACTGTTTACGGGCGTG
GTGCCGATTCTGGTGGAACTGGATGGTGATGTCAATGGTCACAAATTCAGC
GTGCGCGGCGAAGGTGAAGGCGATGCAACCAATGGTAAACTGACGCTGAAG
TTTATTTGCACCACGGGTAAACTGCCGGTTCCGTGGCCGACCCTGGTCACC
ACGCTGACGTATGGTGTTCAGTGTTTCAGTCGTTACCCGGATCACATGAAA
CGCCACGACTTTTTCAAGTCCGCGATGCCGGAAGGTTATGTCCAAGAACGT
ACCATCTCATTTAAAGATGACGGCACCTACAAAACGCGCGCCGAAGTGAAA
TTCGAAGGTGATACGCTGGTTAACCGTATTGAACTGAAAGGCATCGATTTT
AAGGAAGACGGTAATATTCTGGGCCATAAACTGGAATATAACTTCAATTCG
CACAACGTGTACATCACCGCAGATAAGCAGAAGAACGGTATCAAGGCTAAC
TTCAAGATCCGCCATAATGTGGAAGATGGCAGCGTTCAACTGGCCGACCAC
TATCAGCAAAACACCCCGATTGGTGATGGCCCGGTCCTGCTGCCGGACAAT
CATTACCTGAGCACGCAGTCTGTGCTGAGTAAAGATCCGAACGAAAAGCGT
GACCACATGGTCCTGCTGGAATTCGTGACCGCGGCCGGCATCACGCACGGT
ATGGACGAACTGTATAAAGGCTCACATCATCATCATCATCATTAATAA
Sequence for Cav-Tyr:
(SEQ ID NO: 39)
ATGAAAGGTCGTGACTCTGAAGGTCACCTGTACACCGTTCCGATCCGTGAA
GGTAAAGGTGGCGGTAGCGGCAGCAAGGGCGAAGAACTGTTTACGGGCGTG
GTGCCGATTCTGGTGGAACTGGATGGTGATGTCAATGGTCACAAATTCAGC
GTGCGCGGCGAAGGTGAAGGCGATGCAACCAATGGTAAACTGACGCTGAAG
TTTATTTGCACCACGGGTAAACTGCCGGTTCCGTGGCCGACCCTGGTCACC
ACGCTGACGTATGGTGTTCAGTGTTTCAGTCGTTACCCGGATCACATGAAA
CGCCACGACTTTTTCAAGTCCGCGATGCCGGAAGGTTATGTCCAAGAACGT
ACCATCTCATTTAAAGATGACGGCACCTACAAAACGCGCGCCGAAGTGAAA
TTCGAAGGTGATACGCTGGTTAACCGTATTGAACTGAAAGGCATCGATTTT
AAGGAAGACGGTAATATTCTGGGCCATAAACTGGAATATAACTTCAATTCG
CACAACGTGTACATCACCGCAGATAAGCAGAAGAACGGTATCAAGGCTAAC
TTCAAGATCCGCCATAATGTGGAAGATGGCAGCGTTCAACTGGCCGACCAC
TATCAGCAAAACACCCCGATTGGTGATGGCCCGGTCCTGCTGCCGGACAAT
CATTACCTGAGCACGCAGTCTGTGCTGAGTAAAGATCCGAACGAAAAGCGT
GACCACATGGTCCTGCTGGAATTCGTGACCGCGGCCGGCATCACGCACGGT
ATGGACGAACTGTATAAAGGCTCACATCATCATCATCATCATTAATAA

Expression and Purification of sfGFP Constructs.

Cultures of E. coli strain C321.dprfA containing a genomic copy of the pAcFRS.1.t1 synthetase (Amiram et al., Nat Biotechnol 33, 1272-1279 (2015)) under the araBAD promoter and a constitutive tRNA_{CUA} were grown in the presence of 30 μg/ml kanamycin at 34° C. in Luria-Bertani medium (American Bioanalytical, Natick, MA, USA) containing 5 g/L NaCl. Cultures for the expression of azido-containing proteins were supplemented with 1 mMp-azido-L-phenylalanine (Bachem, Bubendorf, Switzerland) and 0.2% arabinose (Fisher Scientific, Hampton, NH, USA) for induction of the orthogonal translation system. GFP expression was induced at OD 0.4-0.6 using 30 ng/ml anhydrotetracycline and allowed to grow overnight. Cells were pelleted prior to freezing at −80° C. Pellets were resuspended in lysis buffer (50 mM Tris pH 8.0, 500 mM NaCl, 0.5 mM EDTA, 0.5 mM EGTA, 10% glycerol) and lysed by ultrasonic disruption (12 cycles of 10 second sonication, separated by 40 second intervals). Clarified lysate was incubated with Ni-NTA agarose (Qiagen, Hilden, Germany) prepared with equilibration buffer (50 mM sodium phosphate, 300 mM NaCl, pH 7.0). Columns were then washed with >10 column volumes (50 mM sodium phosphate, 300 mM NaCl, 5 mM imidazole, pH 7.0) and eluted (50 mM sodium phosphate, 300 mM NaCl, 500 mM imidazole, pH 7.0).

### Phosphoramidate Protein Production.

Cav-Tyr and Cav-pAzF (50 μg) were incubated with agitation overnight with 100 eq. of TNBP at 4° C. in buffer (500 mM Tris pH 8.0) in a total reaction volume of 500 μL. To remove excess of TNBP, samples were buffer exchanged with 50 mM Tris pH 8.0 using Amicon 10 kDa columns. Samples were stored at −20° C. UV deprotection of GFP constructs was performed with either laser irradiation or by exposure to sunlight. In both cases, samples were kept on ice in quartz Hellma Micro absorption cuvettes (5 mm) with a spectral range of 200-2500 nm (Sigma-Aldrich, Kawasaki, Kanagawa Prefecture, Japan). Laser-based deprotection used an Nd:YAG 355 nm laser for 45 s operating at 10 Hz with pulse energy of 10 mJ/cm². For sunlight-based deprotection, cuvettes were exposed to direct sunlight in a water/ice bath.

### SDS-PAGE and Western Blotting.

Blots were transferred to PVDF using the Bio-Rad TransBlot Turbo system according to manufacturer instructions. Membranes were blocked with 5% BSA (Sigma) in TBST before addition of 1:1000 anti-phospho-Caveolin antibody (ab38468, Abcam, Cambridge, UK) in 5% BSA in TBST. After washing, blots were exposed to HRP-conjugated goat anti-rabbit (7074, Cell Signaling, Danvers, MA, USA) with 5% non-fat dry milk omniblock (American Bioanalytical) in TBST. Stripping was performed with Restore Western Blot Stripping Buffer (Thermo Fisher Scientific, Waltham, MA, USA) according to manufacturer instructions. Re-blotting for GFP was performed by blocking with 5% non-fat dry milk omniblock in TBST before the addition of 1 μg/ml mouse anti-GFP (33-2600, Thermo Fisher Scientific). After washing, blots were exposed to HRP-conjugated goat-anti mouse (ab97023, Abcam). Blots were developed using Clarity ECL Western Blotting Substrate (Bio-Rad) and then read and analyzed on GE Amersham Imager 600RGB. Total protein was measured by one of two methods. Stain-Free gel analysis used the Bio-Rad Mini-PROTEAN TGX Stain-Free gel system and Gel Dock EZ Imager system. Band densitometry was performed with system software. Alternatively, protein was measured by SDS-PAGE stained with Coomassie Brilliant Blue (CBB) using a Bio-Rad Gel Dock XR+ running Image Lab 4.0.1.

### Analytical Methods for Protein Analysis.

Protein samples were directly trypsinized overnight at 30 or 37° C. using a trypsin (Promega, Sequencing Grade Modified Trypsin (V51 IC)) to protein ratio of 1:80 or 1:40, prior to either buffer exchange into 100 mM NH₄HCO₃ pH 7.8 or dilution 1:10. The reaction was stopped by adding 5 μL 1% TFA in H₂O. High-resolution mass spectrometry (HRMS) data was collected using an Agilent iFunnel 6550 Quadrupole Time-Of-Flight (QTOF) MS with an electrospray ionization (ESI) source, coupled to an Agilent Infinity 1290 ultra-high-performance liquid chromatography (UHPLC) system with an Agilent Eclipse Plus Cis 1.8 μm, 4.6×50 mm column. Solvents used were (solvent A) water 0.1% formic acid and (solvent B) CH₃CN 0.1% formic acid. An 8 min linear gradient (0% B at 0 min; 95% B at 8 min; 100% B at 10 min) was used and flowrate of 0.7 mL/min. Mass spectra were gathered using Dual Agilent Jet Stream (AJS) ESI in positive mode. The mass range was set from 110 to 1700 m/z with a scan speed of 3 scan/second. The capillary and nozzle voltages were set to 5500 and 2000 V, respectively. The source parameters were set with a gas temperature of 280° C. and a flowrate of 11 L/min, nebulizer at 40 psig, and sheath gas temperature at 350° C. at a flow of 11 L/min. Protein reactions were quantified using calibration curves produced using dilutions of a sample of the protein before each reaction. MS data were acquired with MassHunter Workstation Data Acquistion (Version B.06.01, Agilent Technologies) and analyzed using MassHunter Qualitative Analysis (Version B.07.00, Agilent Technologies).

### Diazotransfer Reaction Using Imidazole-1-Sulfonyl Azide (ISAz).

Diazotransfer reactions with peptides were performed using different proportions of ISAz (200 eq.) in 10×PBS (1.4 M NaCl, 0.1 M phosphate, 0.03 M KCl) pH 7.2. Cav-pAzF protein was reduced using 4 eq. of agarose CL-4B (Sigma) tris(2-carboxyethyl)phosphine at 37° C. for 1.5 h. Reduced Cav-pAzF protein was reacted with 200 eq of ISAz in 10×PBS (1.4 M NaCl, 0.1 M phosphate, 0.03 M KCl) pH 7.2. Diazotransfer reactions were stopped by exchanging the buffer. Samples were subsequently trypsinized as described in Analytical methods for protein analysis. All samples were analyzed by HRMS as described on Analytical methods for protein analysis, using the following 6 min linear gradient (0% B at 0 min; 50% B at 4 min; 95% B at 5 min; 100% B at 6 min) for the analysis of peptide reactions.

“Click” of Cyanine5.5 (Cy5.5) with Cav-GFP and Imaging.

Cu(I)-catalyzed Huisgen azide-alkyne 1,3-dipolar cycloaddition reactions were performed in 35% DMSO, 0.5 mM tris(benzyltriazolylmethyl)amine (THPTA), 0.1 mM CuSO4, 5 mM aminoguanidine hydrochloride, 5 mM sodium ascorbate, 0.15 mM Cy5.5-alkyne and 30 μM of Cav-GFP. THPTA and CuSO4 were premixed for 30 min before the reaction was initiated. The reaction proceeded for 1 h at r.t., and samples were run directly using a Bio-Rad Mini-PROTEAN TGX gel. Imaging was performed with an Amersham Imager 600RGB using the Cy5 filter. Coomassie staining was used to determine total protein content.

### Results

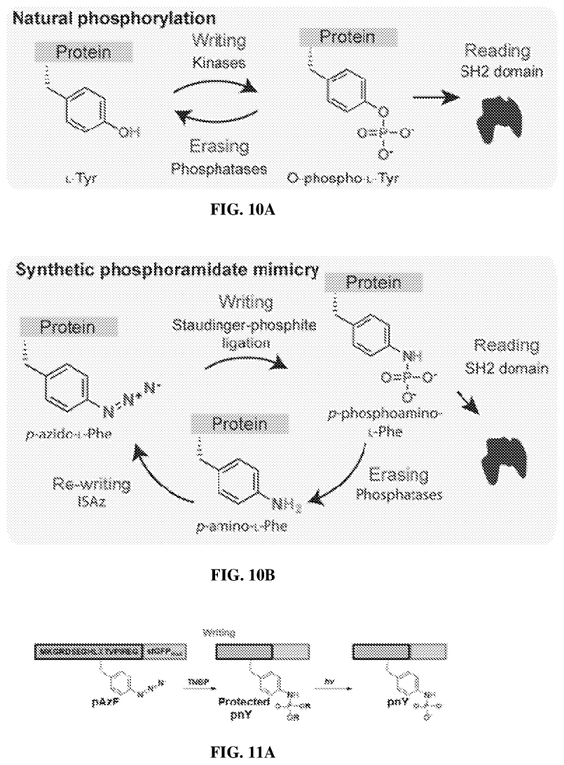

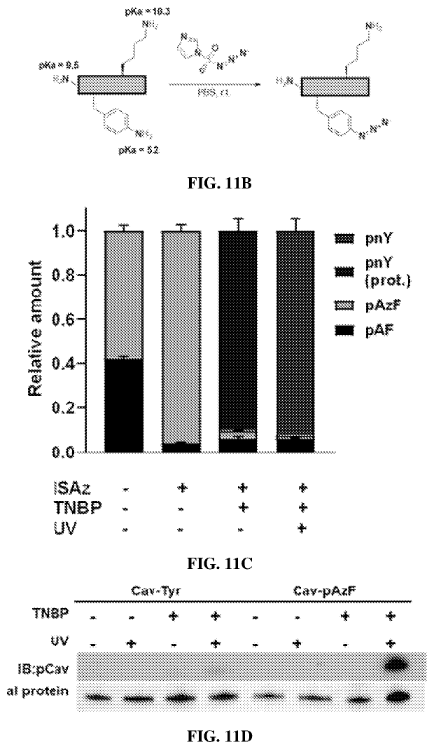

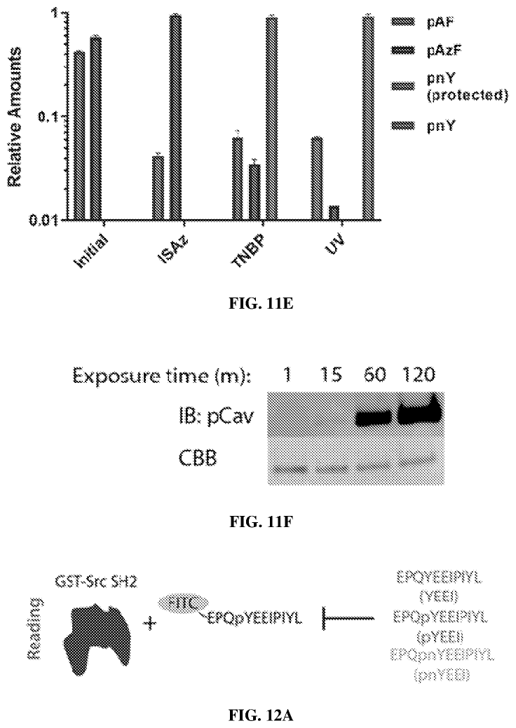

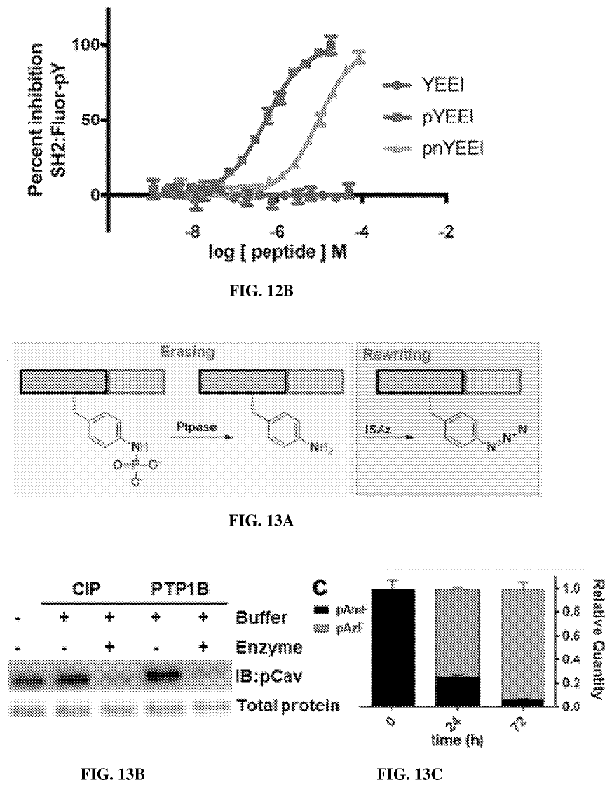

In order to produce and study phosphotyrosine proteins, a reporter protein construct (FIG. 11A) composed of an N-terminal pTyr motif and C-terminal His-tagged super-folder GFP (sfGFP) (P6delacq et al., Nature Biotechnology 24, 79 (2005)) was designed. To select a suitable N-terminal epitope, a tyrosine kinase phosphorylation motif with physiological significance in human cells that also has minimal crosstalk with phosphorylation motifs native to E. coli was used. Caveolin-1 (Cav-1) is a highly regulated plasma membrane protein that is phosphorylated by Src-family kinases and dephosphorylated by protein Tyr phosphatase 1B (PTP1B) in mammalian cells (del Pozo et al., Nat Cell Biol 7, 901-908 (2005), Lee et al., Biochemistry 45, 234-240 (2006)). Known E. coli phosphorylation motifs were reviewed in order to minimize potential host crosstalk with the reporter (Hansen et al., PLoS Pathog 9, e1003403 (2013)) and it was found that the Cav-1 Tyr14 phosphorylation site minimally overlapped with E. coli pTyr motifs and had robust, commercially available antibodies (Hornbeck et al., Nucleic acids research 40, D261-270 (2012)). A fusion protein containing the Cav-1 Tyr14 epitope with either Tyr (Cav(Tyr)GFP) or pAzF (Cav(pAzF)GFP) was purified after expression in the recoded E. coli with the pAcFRS.1.t1 aaRS-tRNA pair derived from M. jannaschii (Amiram et al., Nat Biotechnol 33, 1272-1279 (2015)).

The Staudinger-phosphite ligation, which involves a two-step conversion of pAzF to pnY in alkaline buffered aqueous conditions and room temperature was quantitatively studied (FIG. 11A) (Serwa et al., Angew Chem Int Ed Engl 48, 8234-8239 (2009)). HPLC-MS was used to further evaluate reaction conversions after trypsin digestion. Relative quantities of each tryptic peptide were calculated after correction of ion abundance in each sample using calibration curves. Analysis of purified Cav(pAzF)GFP revealed LC/MS peaks corresponding to both pAzF and its degradation product, p-amino-L-phenylalanine (pAF). This observation is in agreement with prior studies (Wang et al., Nature Chemistry 6, 393 (2014), Young et al., Journal of Molecular Biology 395, 361-374 (2010)), and can be reversed in a regioselective manner using imidazole-1-sulfonyl azide (ISAz; FIG. 11B) (Examples 1-3; Schoffelen et al., Chemical Science 2, 701-705 (2011), (van Dongen et al., Bioconjugate Chemistry 20, 20-23 (2009)). It was found that treatment of proteins with ISAz leads to the recovery of pAzF from −60% to >95% purity (FIGS. 11C and 11E) (see also Examples 1-3).

Next, the protein was treated with tris(4-(2,5,8,11,14-pentaoxahexadecan-16-yloxy)-5-methoxy-2-nitrobenzyl) phosphite (TNBP, below) (100 eq., 16 h), producing Cav(pnY-protected)GFP.

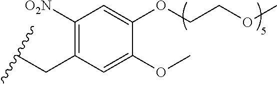

R-group of P(OR)₃, i.e. tris(4-(2,5,8,11,14-pentaoxahexadecan-16-yloxy)-5-methoxy-2-nitrobenzyl) phosphite (TNBP).

Reduction in signal from pAzF was used to estimate the conversions to Cav(pnY-protected)GFP, which indicates efficient conversion after 16 h (FIG. 11C). Finally, laser deprotection (355 nm, 90 s) (Serwa et al., Angew Chem Int Ed Engl 48, 8234-8239 (2009)) was performed, prior to a final buffer exchange purification step to yield the desired Cav(pnY)GFP. HPLC/MS revealed that laser deprotection led to >99% conversion of pnY-protected to unprotected pnY. Collectively, the conversion of pAzF to pnY was 92% in the reporter protein (FIG. 11C). Of note, deprotection of Cav(pnY-protected)GFP was observed with prolonged exposure to sunlight, making this technique accessible without specialized UV-spectrum LEDs and lasers (FIG. 11F). To further confirm the successful conversion of Cav(pAzF)GFP to Cav(pnY)GFP, protein phosphorylation was visualized with phospho-caveolin antibody (FIG. 11D). Only after both Staudinger ligation at pAzF and UV-deprotection was antibody binding readily observed, indicating specificity for Cav(pnY)GFP. No decay in the phosphorylation signal was observed after a two-hour incubation, indicating that this moiety is stable over this time interval. Together, these data indicate that this method for site-specific incorporation of pnY facilitates “writing” at high yield and purity.

### Example 10: Quantification of pnY Binding to the Src SH2 Domain

### Materials and Methods

Preparation of Peptides pnYEEI-Protected and pnYEEI.

Peptides containing pAzF were diluted to 10 mM in 1 M Tris pH 8.0 and combined with 10 eq. of TNBP. The mixture was incubated overnight at r.t. with agitation to obtain the pnYEEI-protected sample. As previously reported,(Serwa et al.) deprotection of the phosphoramidate moiety was performed using 1 mM peptide in 50 mM Tris pH 8.0 with a 355 nm laser for 90 seconds, prior to purification by RP-HPLC (see Analytical methods for peptide purification and analysis).

### Analytical Methods for Peptide Purification and Analysis.

Analytical RP-HPLC data were obtained using an Agilent (Agilent, Santa Clara, CA, USA) 1100 HPLC equipped with G1315A diode array detector, G1312A binary pump, and G1316A column thermostat. A XBridge C₁₈3.5 μm, 4.6×100 mm column was used with 10 mM ammonium acetate pH 9.2 (solvent A) and solvent A/CH₃CN 1:9 (v/v) (solvent B); flow rate of 1 mL/min. Integrations were performed manually using the ChemStation Rev B.04.03 software (Agilent Technologies). Peptide quantification was performed using a standard curve of pTyrEEI peptide and analyzed by RP-HPLC monitored at 210 nm. A linear regression was used to fit the standard curve. RP-HPLC-MS analysis was performed using a Waters system fluidics organizer (SFO), Waters 2998 PDA Detector, Waters 2767 sample manager, Water 515 HPLC pump, 2545 binary gradient module, Waters Autopure SQD2, with MassLynx software version 4.1. A XBridge BEH C₁₈ OBD 130 Å, 5 μm, 10×150 mm column was used; solvents: 0.1% HCO₂H in H₂O (solvent A) and 0.1% HCO₂H in CH₃CN (solvent B); flow rate of 1.4 mL/min. Purification of pnYEEI-protected was performed using the RP-HPLC-MS semi-preparative method. The purification was performed using a 8 min linear gradient as follows: 0% B at 0 min; 10% B at 1 min; 100% B at 8 min. Solvent A, 0.1% CH₃COOH in H₂O; and Solvent B, 0.1% CH₃COOH in CH₃CN. Flow rate was 6.6 mL/min.(Serwa et al.) Purification of pnYEEI was carried out using basic ammonium acetate solvents, as reported previously for the purification of phospholysine-containing peptides, (Bertran-Vicente et al., J Am Chem Soc 136, 13622-13628 (2014b)) in the analytical RP-HPLC system. 10 mM ammonium acetate pH 9.2 (solvent A) and 10% Solvent A, 90% CH₃CN (solvent B) was used on a XBridge C₁₈ 3.5 μm 4.6×100 mm column. The purification was performed using an 8 min linear gradient as follows: 0% B at 0 min; 10% B at 1 min; 100% B at 8 min; flow rate was 1 mL/min. NMR spectra were recorded on a Bruker Avance III D 400 MHz spectrometer with a BBO 5 mm probe operating at ambient temperature. Peptides were dissolved in 10 mM ammonium acetate pH 9.2, 10% D₂O with 3-(trimethylsilyl)propionic-2,2,3,3-d₄ acid (TSP-d4) (D₂O 99.9 atom % D, 0.05 wt. % TSP-d4, sodium salt; Sigma). 31P NMR spectra were recorded using a composite pulse decoupling. 1H NMR spectra were recorded with water suppression. Chemical shifts are reported in parts per million (ppm). TSP-d4 was used as internal reference for proton spectra and these were used to reference the 31P spectra using MestReNova 10.0.1-14719, 2015, Mestrelab Research S.L.

### Fluorescence Polarization Assay.

For the expression of a GST-Src SH2 protein, cultures of E. coli strain BL21 containing pGEX Src-SH2 (Addgene #46510) were grown in the presence of 50 μg/ml carbenicillin at 30° C. until reaching OD 0.4-0.6. They were then induced with 1 mM IPTG and grown overnight. Cells were pelleted prior to freezing at −80° C. Pellets were resuspended in lysis buffer (50 mM Tris, pH 8.0, 500 mM NaCl, 0.5 mM EDTA, 0.5 mM EGTA, 10% glycerol) and then sonicated. Clarified lysate was incubated with Glutathione Sepharose 4B resin, washed (50 mM Tris, pH 8.0, 50 mM NaCl, 10% glycerol) and eluted from resin in elution buffer (20 mM reduced glutathione, 50 mM Tris, pH 8.0, 50 mM NaCl, 10% glycerol, pH 8.0). Glycerol was supplemented to 25% (v/v). Protein was stored at −80° C. Purified GST-Src SH2 was quantified by A280 with an extinction coefficient of 57,675 M⁻¹ cm⁻¹, and diluted to 200 μM in assay buffer (20 mM potassium phosphate pH 7.4, 100 mM NaCl, 2 mM DTT, 0.1% bovine gamma globulin) (Ju et al., Mol Biosyst 9, 1829-1832 (2013), Ju et al., ACS chemical biology (2016)). All experiments were performed using Corning 3673 384-well black non-binding low-volume plates and read with EnVision Green Room after 5-minute incubation at r.t. The plate was calibrated using wells containing only 10 μL 10 nM FITC-pTyrEEI in assay buffer. GST-Src SH2 was diluted in a two-fold dilution series from 200 μM and 5 μL were added to 5 μL of 20 nM FITC-pTyrEEI peptide. K_{d} was established using one-site binding model in GraphPad Prism version 7 for Mac (GraphPad Software, La Jolla, CA, USA). Unlabeled peptides were diluted to 100 μM in assay buffer. Aliquots of pTyrEEI and pnYEEI were set aside for re-analysis of precise concentration using RP-HPLC (analytical) as described earlier and these measurements were done in duplicate. GST-Src SH2 was diluted to 200 nM with 10 nM FITC-pTyrEEI. A full-dissociation control was established with 10 nM peptide in assay buffer. Two-fold dilution series of the three peptides in assay buffer were added to the protein/FITC-pTyrEEI stock in triplicate at 1:1 ratio of the mixtures. Data were normalized using free FITC-pTyrEEI as minimum and FITC-pTyrEEI with GST-Src SH2 as maximum. EC50 for percent inhibition was estimated by plotting log₁₀[agonist] vs. response and fitting a four-parameter variable slope fit in GraphPad Prism.

TABLE 7
Characterization of the peptides synthesized by the manufacturer.

| | | Theoretical | Experimental | |
|---|---|---|---|---|
| Peptide | | m/z [M + | m/z [M + | Purity |
| ID | Sequence | H]⁺ | H]⁺ | (%) |
| YEEI | EPQYEEIPIYL (SEQ | 1393.7 | 1394.3 | >99 |
| | ID NO: 41) | | | |
| pTyrEEI | EPQ-[phospho-L- | 1473.7 | 1474.7 | >99 |
| | tyrosine]-EEIPIYL | | | |
| | SEQ ID NO: 42) | | | |
| ZEEI | EPQ-[p-azido-L- | 1418.7 | 1419.4^{a} | >99 |
| | phenylalanine]- | | | |
| | EEIPIYL SEQ ID | | | |
| | NO: 43) | | | |
| FITC- | FITC-(AHA)-EPQ- | 1975.8 | 1976.0 | >99 |
| pTyrEEI | [phospho-L- | | | |
| | tyrosine]-EEIPIYL | | | |
| | SEQ ID NO: 44) | | | |

^{a}For ZEEI, the manufacturer observed a loss of N₂ in MALDI-TOF, which has been noted in other studies of azido proteins by mass spectrometry (Amiram, et al., Nat Biotechnol 33, 1272-1279 (2015)). The presence of the azido group was confirmed by RP-HPLC-MS (analytical method described in Table 9);

TABLE 8
Summary data of the characterization
of peptides used in this study.

| | Chroma- | | Theoretical | Experimental | |
|---|---|---|---|---|---|
| Peptide | tographic | t_{R} | m/z [M + | m/z^{e} [M + | Yield |
| ID | method^{a} | (min) | 2H]²⁺ | 2H]²⁺ | (%) |
| pnYEEI- | HPLC-MS | 8.3^{b} | 1152.02 | 1152.97 | 40 |
| protected | | | | | |
| pnYEEI | RP-HPLC | 4.7^{c} | 736.83 | 737.28 | 36^{f} |

^{a}HPLC-MS method refers to RP-HPLC-MS (semi-preparative), RP-HPLC refers to RP-HPLC (analytical), and LC-HRMS refers to UHPLC-QTOF MS.
^{b}Linear RP-HPLC-MS (semi-preparative) gradient: 2% B at 0 min; 32% B at 1 min; 40% B at 9 min; 100% B at 11 min.
^{c}Linear RP-HPLC (analytical) gradient: 2% B at 0 min; 20% B at 12 min; 60% B at 15 min; 98% B at 16 min.
^{d}Linear gradient: 0% B at 0 min; 50% B at 4 min; 95% B at 5 min; 100% B at 6 min.
^{e}Determined using an RP-HPLC-MS (analytical) method.
^{f}Yield calculated using the peptide P(OR)₂ YEEI as starting material.
^{g}Not applicable.

TABLE 9
Calculated pKa values for pTyr and pnY used in this
study. Values were calculated using MarvinSketch.

| Amino Acid | pKₐ₂ |
|---|---|
| pTyr | 7.4 |
| pnY | 6.9 |

### Results

To functionally investigate the biochemical attributes of the pTyr mimetic, the binding affinity between a pnY-containing motif and a known binding SH2 domain partner was investigated. SH2 domains are important for in vivo regulation of pTyr signaling and have been previously shown to have an exquisite ability to discriminate between pTyr, sY, and pCMF (Burke et al., Biochemistry 33, 6490-6494 (1994), Ju et al., Mol Biosyst 9, 1829-1832 (2013), Tong et al., J Biol Chem 273, 20238-20242 (1998)). A well-characterized peptide derived from the hamster polyomavirus middle T antigen was utilized in a series of fluorescence polarization (FP) assays to compare the impact of different chemical moieties (Tyr, pTyr, and pnY) on binding to the Src SH2 domain (Songyang et al., Cell 72, 767-778 (1993), Waksman et al., Cell 72, 779-790 (1993)). For these experiments, a small library of peptides containing Tyr, pTyr, and pAzF in the variable residue position of the EPQxEEIPIYL (SEQ ID NO:40) motif (Table 7 and 8) was synthesized. The pnY peptide was then produced from the pAzF precursor using analogous methods to those used for proteins (Bertran-Vicente et al., Journal of the American Chemical Society 136, 13622-13628 (2014a), Serwa et al., Angew Chem Int Ed Engl 48, 8234-8239 (2009)). All peptides used in this study were characterized by RP-HPLC and HPLC-MS (Table 8). 31P-NMR was recorded for peptides containing a phosphorous atom. The pTyr and pnY peptides had similar HPLC retention times, but distinguishable chemical shifts on 31P-NMR (−3.9 and −0.3 ppm, respectively).

The binding affinity of a pTyr peptide labeled with fluorescein isothiocyanate (FITC) to Src SH2 was established. This peptide displayed a Kd of 0.28 μM, which is in good agreement with prior studies that indicated sub-micromolar affinities (Ladbury et al., Proceedings of the National Academy of Sciences 92, 3199 (1995)). EC50 was calculated from competition assays between FITC-labeled pTyr peptide, the Src SH2 domain, and peptides containing Tyr, pnY, or pTyr (FIG. 12A). No increase in unbound FITC-pTyr peptide with the addition of Tyr peptide was detected, while the pTyr peptide had an EC50 of 0.74 μM (FIG. 12B). An EC50 of 12.8 μM for pnY peptide was observed, 17-fold weaker than that of pTyr. The difference in the pKa of the pnY phosphate group may contribute to the difference in binding (Table 9) (Burke et al., Biochemistry 33, 6490-6494 (1994)). When compared with other pTyr mimetics that have been assayed in similar SH2 binding experiments, the binding affinity of pnY is 9-fold more potent than sY (Ju et al., Mol Biosyst 9, 1829-1832 (2013), 27-fold more potent than pCMF (Tong et al., J Biol Chem 273, 20238-20242 (1998), and 3-fold weaker than Pmp (Burke et al., Biochemistry 33, 6490-6494 (1994)). Notably, these results demonstrate that pnY can be “read” by SH2 domains similarly to pTyr.

### Example 11: Phosphatases Recognize and Dephosphorylate pnY

### Materials and Methods

### Phosphatase Assays.

For calf intestinal phosphatase (CIP), protein was diluted in CutSmart buffer (New England Biolabs), pH 7.9. Different amounts of CIP were added per reaction. Negative controls were diluted in the same buffer, but no CIP was added. Samples were incubated at 25° C. for 15 minutes before running on SDS-PAGE and Western blotting. For protein tyrosine phosphatase 1B (PTP1B) assays, proteins were diluted in a buffer containing 50 mM Tris pH 7.9, 50 mM NaCl, 3 mM DTT, and 0.1% BSA. Recombinant PTP1B (Millipore, Billerica, MA, USA) was added and samples were incubated at 25° C. for 15 min before running on SDS-PAGE and subsequent Western blotting.

### Results

Given the importance of dephosphorylation in signal transduction, experiments were designed to evaluate the recognition and dephosphorylation activity of two phosphatases, calf-intestinal alkaline phosphatase (CIP) and protein Tyr phosphatase 1B (PTP1B) on pnY (FIG. 15A). CIP is known to dephosphorylate a wide variety of substrates including DNA and proteins (Swarup et al., J Biol Chem 256, 8197-8201 (1981)). PTP1B is a pTyr-specific phosphatase and critical regulator of Tyr kinase signaling in vivo, which in humans is encoded by the PTPN1 gene. The Cav(pnY)GFP reporter was exposed to both phosphatases for a range of time-points (5 to 120 minutes) and enzyme gradients (0.001 to 10 units per reaction). Cav(pnY)GFP was stable in buffered conditions, but dephosphorylated in a time and concentration dependent fashion (FIG. 15B), indicating that pnY is specifically “erased” by phosphatases.

### Example 12: Re-Writing pnY after Phosphate Hydrolysis

One notable difference between pTyr and the pnY mimetic is that the dephosphorylation product of the former is Tyr whereas the latter is pAF. Earlier work has shown that pAF is not readily phosphorylated (Wang and Cole, J Am Chem Soc 123, 8883-8886 (2001)) indicating that this technique may offer a unique and complementary biologically irreversible probe for the study of phosphorylation-dephosphorylation dynamics. The ability to utilize ISAz to selectively restore pAzF from pAF (see also Examples 1-3) present in pnY-proteins that have been dephosphorylated was investigated. Treatment of fully reduced (or hydrolyzed from pnY) Cav(pAF)GFP with ISAz facilitated the recovery of pAzF, and thus its reusability-25% of pAF tryptic peptide signal was present at 24 h, and only 7% at 72 h (FIG. 15C). The corresponding increase in pAzF-peptide MS signal was observed. These results demonstrate that, in addition to improving pAzF yield in expressed proteins, ISAz can be used to “re-write” pAzF from pAF.

Examples 9-12 illustrate a robust system used to produce proteins with pnY, a pTyr mimetic, at specific sites. Results show that pnY is recognized robustly by SH2 domains and readily dephosphorylated by phosphatases. In sum, these data demonstrate that this approach for pnY incorporation facilitates synthetic control over the phosphorylation cycle, including “writing”, “reading”, “erasing”, and “re-writing”. With these attributes, pnY-based mimicry offers significant advantages over current platforms for deciphering the extensive pTyr-mediated interactions within the cellular wiring diagram (Hunter, Curr Opin Cell Biol 21, 140-146 (2009)). First, E. coli recoding, enhanced OTSs, and ISAz-treatment offers significantly improved yields and purity of pAzF incorporated into protein. It is believed, that when combined with the present findings, the tools are available to study the combinatorics of multi-site tyrosine phosphorylation events, like those found on the catalytic domains of receptor Tyr kinases (Hornbeck et al., Nucleic acids research 40, D261-270 (2012)). Additionally, there is increasing appreciation for the importance of dynamic, widespread Tyr phosphorylation on extracellular domains and secreted proteins, but the upstream kinases are either unknown or have low activity in vitro (Bordoli et al., Cell 158, 1033-1044 (2014)). Given that Staudinger ligations on the cell surfaces of mammalian systems are feasible (Prescher et al., Nature 430, 873-877 (2004)), this phosphoramidate chemistry may provide a valuable route to investigating extracellular Tyr phosphorylation with the goal of elucidating new mechanisms of cellular communication. This pTyr mimetic may be utilized in the discovery of molecules capable of protein binding in specific phosphorylation states, offering opportunities to target signaling networks under selective conditions. This platform provides an effective and straightforward approach for the study of pTyr-mediated signaling. In addition, the “re-writing” capability of ISAz on the chemical cycle of the pnY moiety facilitates a greater versatility on the experimental design. Arrays composed of peptide and protein phospho-isoforms can be utilized in comprehensive analyses of phosphorylated-mediated protein-protein interactions or potential drug development (e.g., inhibitors of such interactions).

Pol Arranz-Gibert, et al., “Chemoselective restoration of para-azido-phenylalanine at multiple sites in proteins,” Cell Chemical Biology, 29, 1-7, (2021) (doi.org/10.1016/j.chembiol.2021.12.002) all supplementary information associated therewith, and Vanderschuren, et al., “Tuning protein half-life in mouse using sequence-defined biopolymers functionalized with lipids,” PNAS, 119, e2103099119, pages 1-9 (2022) (doi.org/10.1073/pnas.2103099119), and all supplementary information associated therewith is specifically incorporated by reference herein in its entirety.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of restoring one or more reduced or degraded para-azido-phenylalanine (pAzF) residues in a polypeptide in need thereof comprising contacting the polypeptide with an effective amount of imidazole-1-sulfonyl azide (ISAz) to restore one or more of the reduced or degraded pAzF residues to pAzF therein.

2. The method of claim 1, wherein the contacting occurs under aqueous conditions.

3. The method of claim 1, wherein the contacting occurs in the absence of organic solvents.

4. The method of claim 1, wherein the conditions are not effective to limit or prevent the conversion of amines at the N-terminus and/or lysine residues to azides.

5. The method of claim 1 wherein the contacting occurs in pH of between about 6.0 and about 8.5 inclusive, or between about 6.5 and about 7.6 inclusive, or between about 7.0 and about 7.5 inclusive, or about 7.2, or 7.2

6. The method of claim 1, wherein the ISAz is in about 2 to about 500 inclusive, or between about 20 and 250 inclusive, or about 200, or 200 equivalents per molecule.

7. The method of claim 1, wherein the contacting is carried out for about 1 to about 150 hours, or about 2 to about 100 hours, or about 5 to about 90 hours, or about 10 to about 72 hours, or about 42, 72, or 90 hours.

8. The method of claim 1, wherein the polypeptide comprises between 1 and 500 residues that are either pAzF or reduced or degraded pAzF.

9. The method of claim 8, wherein at least 10, 15, 20, 25, 30, 35, 40, 45, 50, 55, 60, 65, 70, 75, 80, 85, 90, or 95 percent of the between 1 and 500 residues are reduced or degraded pAzF prior to the contacting.

10. The method of claim 9, at least 95, 90, 85, 80, 75, 70, 65, 60, 55, or 50 percent of the between 1 and 500 residues are pAzF after the contacting.

11.-19. (canceled)

20. The method of claim 1, further comprising modifying the pAzF residues to include one or more moieties conjugated thereto.

21. The method of claim 20, wherein the modifying comprises a copper-catalyzed azide-alkyne cycloaddition (“click”), strain promoted azide-alkyne cycloaddition, or Staudinger ligation photocrosslinking.

22. The method of claim 20, wherein the moiety is a lipid.

23.-26. (canceled)

27. A method of testing the activity of a putative phosphatase comprising
(i) contacting a putative phosphatse with a polypeptide comprising one or more comprising one or more phosphoramidate (pnY) residues;
(ii) optionally selecting the phosphatase when it dephosphorylates pnY to form p-amino-phenylalanine (pAF); and
(iii) converting the pAF to pAzF according to the method of claim 1.

28. The method of claim 27, wherein the polypeptide comprising one or more phosphoramidate (pnY) residues is made according to a method comprising carrying out a Staudinger-phosphite ligation reaction on a pre-cursor polypeptide comprising one or more pAzF residues.

29. The method of claim 28, wherein the Staudinger-phosphite ligation reaction comprises contacting the polypeptide with an effective amount of tris(4-(2,5,8,11,14-pentaoxahexadecan-16-yloxy)-5-methoxy-2-nitrobenzyl) phosphite (TNBP).

30. The method of claim 28 comprising a deprotection reaction.

31. The method of claim 30, wherein the deprotection reaction comprises exposing the polypeptide to UV light.

32. The method of claim 31, wherein the UV light is from a laser, LED, or sunlight.

33.-40. (canceled)

41. A polypeptide manufactured according to the method of claim 1.

42.-48. (canceled)


SEQUENCE LISTING (44 sequences)

SEQ ID NO: 1 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 2 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 3 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 4 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKCPEKEGGDLTVNSYEELESLFKNKELHPMRLKNAVAEELIKILEPIRKRL

SEQ ID NO: 5 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 6 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKCPEKEGGDLTVNSYEELESLFKNKELHPMRLKNAVAEELIKILEPIRKRL

SEQ ID NO: 7 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 8 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 9 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKCPEKEGGDLTVNSYEELESLFKNKELHPMRLKNAVAEELIKILEPIRKRL

SEQ ID NO: 10 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNVMHYDGVDVYVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 11 - protein, 307 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNVMHYDGVDVYVGGMEQRKIHMLARELLPKKVVCIHNPVLTGLDGEGKMSSSKGNFIAVDDSPEEIRAKIKKAYCPAGVVEGNPIMMEIAKYFLEYPLTIKGPEKFGGDLTVNSYEELESLFKNKELHPMRLKNAVAEELIKILEPIRKRL

SEQ ID NO: 12 - protein, 307 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNVMHYDGVDVYVGGMEQRKIHMLARELLPKKVVCIHNPVLTGLDGEGKMSSSKGNFIAVDDSPEEIRAKIKKAYCPAGVVEGNPIMMEIAKYFLEYPLTIKCPEKEGGDLTVNSYEELESLFKNKELHPMRLKNAVAEELIKILEPIRKRL

SEQ ID NO: 13 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSTYMLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 14 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSTYMLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 15 - protein, 306 aa, Artificial Sequence
  synthetic polypeptide
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSTYMLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKCPEKEGGDLTVNSYEELESLFKNKELHPMRLKNAVAEELIKILEPIRKRL

SEQ ID NO: 16 - skipped

SEQ ID NO: 17 - DNA, 77 bp, Artificial Sequence
  synthetic polynucleotide
ccggcggtagttcagcagggcagaacggcggactctaaatccgcatggcaggggttcaaatcccctccgccggacca

SEQ ID NO: 18 - protein, 5 aa, Artificial Sequence
  synthetic polypeptide
  MISC_FEATURE (1)..(5): residues 1-5 are present “n” number of times, wherein “n” is an integer from 1 to 500, or more than 500
  MISC_FEATURE (4)..(4): “X” is pAzF
VPGXG

SEQ ID NO: 19 - protein, 30 aa, Artificial Sequence
  synthetic polypeptide
  MISC_FEATURE (1)..(15): residues 1-15 are present “y” times, wherein “y” is an integer from 1 to 500, or more than 500
  MISC_FEATURE (14)..(14): “X” is pAzF
  MISC_FEATURE (16)..(30): residues 16-30 are present “z” times, wherein “z” is an integer from 1 to 500, or more than 500
VPGGGVPGAGVPGXGVPGGGVPGAGVPGYG

SEQ ID NO: 20 - protein, 6 aa, Artificial Sequence
  synthetic polypeptide
MSKGPG

SEQ ID NO: 21 - protein, 5 aa, Artificial Sequence
  synthetic polypeptide
PGGGG

SEQ ID NO: 22 - protein, 239 aa, Artificial Sequence
  synthetic polypeptide
SKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGS

SEQ ID NO: 23 - DNA, 813 bp, Artificial Sequence
  synthetic polynucleotide
atgaaaggtcgtgactctgaaggtcacctgtagaccgttccgatccgtgaaggtaaaggtggcggtagcggcagcaagggcgaagaactgtttacgggcgtggtgccgattctggtggaactggatggtgatgtcaatggtcacaaattcagcgtgcgcggcgaaggtgaaggcgatgcaaccaatggtaaactgacgctgaagtttatttgcaccacgggtaaactgccggttccgtggccgaccctggtcaccacgctgacgtatggtgttcagtgtttcagtcgttacccggatcacatgaaacgccacgactttttcaagtccgcgatgccggaaggttatgtccaagaacgtaccatctcatttaaagatgacggcacctacaaaacgcgcgccgaagtgaaattcgaaggtgatacgctggttaaccgtattgaactgaaaggcatcgattttaaggaagacggtaatattctgggccataaactggaatataacttcaattcgcacaacgtgtacatcaccgcagataagcagaagaacggtatcaaggctaacttcaagatccgccataatgtggaagatggcagcgttcaactggccgaccactatcagcaaaacaccccgattggtgatggcccggtcctgctgccggacaatcattacctgagcacgcagtctgtgctgagtaaagatccgaacgaaaagcgtgaccacatggtcctgctggaattcgtgaccgcggccggcatcacgcacggtatggacgaactgtataaaggctcacatcatcatcatcatcattaataa

SEQ ID NO: 24 - DNA, 1203 bp, Artificial Sequence
  synthetic polynucleotide
atgagcaaaggtcccggggttccgggtggcggcgtgccgggcgcaggtgttccgggttatggtgtgccgggcggcggtgtcccgggtgctggtgtgccgggctacggtgtcccgggtggcggtgttccgggcgctggtgtcccgggttatggtgtcccgggtggcggtgttccgggtgcaggcgttccgggttacggcgtgccgggcggcggtgttccgggtgctggtgtgccgggctatggtgtcccgggtggcggtgtgccgggcgcaggtgtcccgggttacggtgttccgggcggcggtgtcccgggtgcaggtgtgccgggctatggtgttccgggtggcggggtgccgggcgctggtgttccgggttatggtgtgccgggcggcggtgtcccgggtgcaggtgtgccgggctacggtgtcccgggtggcggtgttccgggcgcaggtgtcccgggttatgggcccggcggtgggggcagcaagggcgaagaactgtttacgggcgtggtgccgattctggtggaactggatggtgatgtcaatggtcacaaattcagcgtgcgcggcgaaggtgaaggcgatgcaaccaatggtaaactgacgctgaagtttatttgcaccacgggtaaactgccggttccgtggccgaccctggtcaccacgctgacgtatggtgttcagtgtttcagtcgttacccggatcacatgaaacgccacgactttttcaagtccgcgatgccggaaggttatgtccaagaacgtaccatctcatttaaagatgacggcacctacaaaacgcgcgccgaagtgaaattcgaaggtgatacgctggttaaccgtattgaactgaaaggcatcgattttaaggaagacggtaatattctgggccataaactggaatataacttcaattcgcacaacgtgtacatcaccgcagataagcagaagaacggtatcaaggctaacttcaagatccgccataatgtggaagatggcagcgttcaactggccgaccactatcagcaaaacaccccgattggtgatggcccggtcctgctgccggacaatcattacctgagcacgcagtctgtgctgagtaaagatccgaacgaaaagcgtgaccacatggtcctgctggaattcgtgaccgcggccggcatcacgcacggtatggacgaactgtataaaggctcataa

SEQ ID NO: 25 - DNA, 1203 bp, Artificial Sequence
  synthetic polynucleotide
atgagcaaaggtcccggggttccgggtggcggcgttccgggcgcaggtgttccgggttagggtgtcccgggcggcggtgtcccgggtgctggtgttccgggctacggtgtcccgggtggcggtgttccgggcgctggtgtcccgggttagggtgtcccgggtggcggtgttccgggtgcaggcgttccgggttacggcgtcccgggcggcggtgttccgggtgctggtgttccgggctagggtgtcccgggtggcggtgtcccgggcgcaggtgtcccgggttacggtgttccgggcggcggtgtcccgggtgcaggtgttccgggctagggtgttccgggtggcggggtcccgggcgctggtgttccgggttatggtgttccgggcggcggtgtcccgggtgcaggtgtcccgggctagggtgtcccgggtggcggtgttccgggcgcaggtgtcccgggttatgggcccggcggtgggggcagcaagggcgaagaactgtttacgggcgtggtgccgattctggtggaactggatggtgatgtcaatggtcacaaattcagcgtgcgcggcgaaggtgaaggcgatgcaaccaatggtaaactgacgctgaagtttatttgcaccacgggtaaactgccggttccgtggccgaccctggtcaccacgctgacgtatggtgttcagtgtttcagtcgttacccggatcacatgaaacgccacgactttttcaagtccgcgatgccggaaggttatgtccaagaacgtaccatctcatttaaagatgacggcacctacaaaacgcgcgccgaagtgaaattcgaaggtgatacgctggttaaccgtattgaactgaaaggcatcgattttaaggaagacggtaatattctgggccataaactggaatataacttcaattcgcacaacgtgtacatcaccgcagataagcagaagaacggtatcaaggctaacttcaagatccgccataatgtggaagatggcagcgttcaactggccgaccactatcagcaaaacaccccgattggtgatggcccggtcctgctgccggacaatcattacctgagcacgcagtctgtgctgagtaaagatccgaacgaaaagcgtgaccacatggtcctgctggaattcgtgaccgcggccggcatcacgcacggtatggacgaactgtataaaggctcataa

SEQ ID NO: 26 - DNA, 1203 bp, Artificial Sequence
  synthetic polynucleotide
atgagcaaaggtcccggggttccgggtggcggcgtgccgggcgcaggtgttccgggttagggtgtgccgggcggcggtgtcccgggtgctggtgtgccgggctagggtgtcccgggtggcggtgttccgggcgctggtgtcccgggttagggtgtcccgggtggcggtgttccgggtgcaggcgttccgggttagggcgtgccgggcggcggtgttccgggtgctggtgtgccgggctagggtgtcccgggtggcggtgtgccgggcgcaggtgtcccgggttagggtgttccgggcggcggtgtcccgggtgcaggtgtgccgggctagggtgttccgggtggcggggtgccgggcgctggtgttccgggttagggtgtgccgggcggcggtgtcccgggtgcaggtgtgccgggctagggtgtcccgggtggcggtgttccgggcgcaggtgtcccgggttaggggcccggcggtgggggcagcaagggcgaagaactgtttacgggcgtggtgccgattctggtggaactggatggtgatgtcaatggtcacaaattcagcgtgcgcggcgaaggtgaaggcgatgcaaccaatggtaaactgacgctgaagtttatttgcaccacgggtaaactgccggttccgtggccgaccctggtcaccacgctgacgtatggtgttcagtgtttcagtcgttacccggatcacatgaaacgccacgactttttcaagtccgcgatgccggaaggttatgtccaagaacgtaccatctcatttaaagatgacggcacctacaaaacgcgcgccgaagtgaaattcgaaggtgatacgctggttaaccgtattgaactgaaaggcatcgattttaaggaagacggtaatattctgggccataaactggaatataacttcaattcgcacaacgtgtacatcaccgcagataagcagaagaacggtatcaaggctaacttcaagatccgccataatgtggaagatggcagcgttcaactggccgaccactatcagcaaaacaccccgattggtgatggcccggtcctgctgccggacaatcattacctgagcacgcagtctgtgctgagtaaagatccgaacgaaaagcgtgaccacatggtcctgctggaattcgtgaccgcggccggcatcacgcacggtatggacgaactgtataaaggctcataa

SEQ ID NO: 27 - protein, 15 aa, Artificial Sequence
  synthetic polypeptide
  MISC_FEATURE (6)..(6): “X” is pAF
AGVPGXGVPGGGVPG

SEQ ID NO: 28 - protein, 15 aa, Artificial Sequence
  synthetic polypeptide
  MISC_FEATURE (6)..(6): “X” is pAzF.
AGVPGXGVPGGGVPG

SEQ ID NO: 29 - protein, 15 aa, Artificial Sequence
  synthetic polypeptide
AGVPGYGVPGGGVPG

SEQ ID NO: 30 - protein, 13 aa, Artificial Sequence
  synthetic polypeptide
  MISC_FEATURE (7)..(7): “X” is pAF.
DSEGHLXTVPIRE

SEQ ID NO: 31 - protein, 13 aa, Artificial Sequence
  synthetic polypeptide
  MISC_FEATURE (7)..(7): “X” is pAzF.
DSEGHLXTVPIRE

SEQ ID NO: 32 - protein, 15 aa, Artificial Sequence
  synthetic polypeptide
  MISC_FEATURE (9)..(9): “X” is either tyrosine or pAzF.
VPGAGVPGXGVPGGG

SEQ ID NO: 33 - protein, 161 aa, Artificial Sequence
  synthetic polypeptide
MSKGPGVPGGGVPGAGVPGYGVPGGGVPGAGVPGYGVPGGGVPGAGVPGYGVPGGGVPGAGVPGYGVPGGGVPGAGVPGYGVPGGGVPGAGVPGYGVPGGGVPGAGVPGYGVPGGGVPGAGVPGYGVPGGGVPGAGVPGYGVPGGGVPGAGVPGYGPGGGG

SEQ ID NO: 34 - protein, 161 aa, Artificial Sequence
  synthetic polypeptide
  MISC_FEATURE (20)..(20): “X” is pAzF.
MSKGPGVPGGGVPGAGVPGXGVPGGGVPGAGVPGYGVPGGGVPGAGVPGYGVPGGGVPGAGVPGYGVPGGGVPGAGVPGYGVPGGGVPGAGVPGYGVPGGGVPGAGVPGYGVPGGGVPGAGVPGYGVPGGGVPGAGVPGYGVPGGGVPGAGVPGYGPGGGG

SEQ ID NO: 35 - protein, 160 aa, Artificial Sequence
  synthetic polypeptide
  misc_feature (20)..(20): “X” = pAzF.
  misc_feature (49)..(49): “X” = pAzF.
  misc_feature (79)..(79): “X” = pAzF.
  misc_feature (109)..(109): “X” = pAzF.
  misc_feature (139)..(139): “X” = pAzF.
MSKGPGVPGGGVPGAGVPGXGVPGGGVPGAGVPGYVPGGGVPGAGVPGXGVPGGGVPGAGVPGYGVPGGGVPGAGVPGXGVPGGGVPGAGVPGYGVPGGGVPGAGVPGXGVPGGGVPGAGVPGYGVPGGGVPGAGVPGXGVPGGGVPGAGVPGYGPGGGG

SEQ ID NO: 36 - protein, 161 aa, Artificial Sequence
  synthetic polypeptide
  MISC_FEATURE (20)..(20): “X” is pAzF.
  MISC_FEATURE (35)..(35): “X” is pAzF.
  MISC_FEATURE (50)..(50): “X” is pAzF.
  MISC_FEATURE (65)..(65): “X” is pAzF.
  MISC_FEATURE (80)..(80): “X” is pAzF.
  MISC_FEATURE (95)..(95): “X” is pAzF.
  MISC_FEATURE (110)..(110): “X” is pAzF.
  MISC_FEATURE (125)..(125): “X” is pAzF.
  MISC_FEATURE (140)..(140): “X” is pAzF.
  MISC_FEATURE (155)..(155): “X” is pAzF.
MSKGPGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGPGGGG

SEQ ID NO: 37 - DNA, 1203 bp, Artificial Sequence; identical to SEQ ID NO: 24
  synthetic polynucleotide

SEQ ID NO: 38 - DNA, 813 bp, Artificial Sequence; identical to SEQ ID NO: 23
  synthetic polynucleotide

SEQ ID NO: 39 - DNA, 813 bp, Artificial Sequence
  synthetic polynucleotide
atgaaaggtcgtgactctgaaggtcacctgtacaccgttccgatccgtgaaggtaaaggtggcggtagcggcagcaagggcgaagaactgtttacgggcgtggtgccgattctggtggaactggatggtgatgtcaatggtcacaaattcagcgtgcgcggcgaaggtgaaggcgatgcaaccaatggtaaactgacgctgaagtttatttgcaccacgggtaaactgccggttccgtggccgaccctggtcaccacgctgacgtatggtgttcagtgtttcagtcgttacccggatcacatgaaacgccacgactttttcaagtccgcgatgccggaaggttatgtccaagaacgtaccatctcatttaaagatgacggcacctacaaaacgcgcgccgaagtgaaattcgaaggtgatacgctggttaaccgtattgaactgaaaggcatcgattttaaggaagacggtaatattctgggccataaactggaatataacttcaattcgcacaacgtgtacatcaccgcagataagcagaagaacggtatcaaggctaacttcaagatccgccataatgtggaagatggcagcgttcaactggccgaccactatcagcaaaacaccccgattggtgatggcccggtcctgctgccggacaatcattacctgagcacgcagtctgtgctgagtaaagatccgaacgaaaagcgtgaccacatggtcctgctggaattcgtgaccgcggccggcatcacgcacggtatggacgaactgtataaaggctcacatcatcatcatcatcattaataa

SEQ ID NO: 40 - protein, 11 aa, Artificial Sequence
  synthetic polypeptide
  MISC_FEATURE (4)..(4): “X” is Tyr, pTyr, or pAzF.
EPQXEEIPIYL

SEQ ID NO: 41 - protein, 11 aa, Artificial Sequence
  synthetic polypeptide
EPQYEEIPIYL

SEQ ID NO: 42 - protein, 11 aa, Artificial Sequence
  synthetic polypeptide
  MISC_FEATURE (4)..(4): “X” is phospho-L-tyrosine.
EPQXEEIPIYL

SEQ ID NO: 43 - protein, 11 aa, Artificial Sequence
  synthetic polypeptide
  MISC_FEATURE (4)..(4): “X” is p-azido-L-phenylalanine.
EPQXEEIPIYL

SEQ ID NO: 44 - protein, 14 aa, Artificial Sequence
  synthetic polypeptide
  MISC_FEATURE (1)..(1): conjugated to FITC.
  MISC_FEATURE (7)..(7): “X” is phospho-L-tyrosine.
AHAEPQXEEIPIYL